SEQUENCE LISTING (43 sequences)

SEQ ID NO: 1 - DNA, 1836 bp, Fragaria sp.
  source 1..1836: mol_type = other DNA
atgcttctgaatttgttatgctgttggtttctcataggcgcagtgctagatgtagatattattgtgcatggagaatcagttggccctgaaaattgcacagaggttcgatgtaaacggcatggccctgtgatccgattcccatttcgactgaaaggaatgcaaccactccattgcggctacccgggctttgatctgtcatgcaccacggacaaccagactgtgctccagatcaactcatcatcagctgacaagttcttggttaaaaggattgactacactgctcaggaaattgaaatatatctttataatagtcactatgattatttcaatgatttccagtatatctgtattcctagacagattttcgagctcagttcctctccattcgaattctcagacgcccaaagaaaaactaactataccatattcagttgtccaccgtcagataaaagagatcggtctccatccttctgtccactggcaaaactgagcccttgccttggcaatcatagcaacaggatgtatgctcttcagaatggatgtgcagagcttaaatacatgccccaactggtgtcatgtaccaaggtggaagattacatgtcagtaccatctgtctggatgcagcctgtgaagctcaaatggaccagaccatcgtgtaaacgttgcgaagagatgggcaagaaatgtggatttctgaatcaagttgcagatgatcagaaaactgaagaaactcaatgtttagaggtgcctaaaggtcctaatcgtggaaaacgaatcttggagatattaggcatttgtgcacttgctaccgttctcacaggagtggctactgtaatatattatgtgtatagttctaagaagacagaagaagaaaatcaactgagaattgaaaagtttttggatgattacagagctctcaaacccagtagatatgcctatggtgatattaggcggataacaaatcaatttgaggacaagttgggtcagggagcttatggaaccgtgtacaaaggaaagctttcctctgaattacttgttgctgtgaaaatcctcgacaattcacacgagaagggggaagatttcattaatgaggtgggaacaatgggtctcatccaccatgtcaatgtggttcgcttggttggcttttgcgctgatggattcatacgagctcttatatatgagtatttaccaaatggctcattgcagaattacttgtcatctgcagatgataagaactcatttcttggttgggaaaagctgcaagatattgctttaggtatagctaaaggaatcgaatatcttcatcaagggtgtgaccagagaattctgcattttgatatcaaacctagtaacattttgctagaccaagagttcactccaaaagtttctgattttggtctagccaagttgtgctctaaggatcagagtgcaatatccatgactacagctagggggaccatgggctacattgcaccagaagtgttctctaggaactttggaaacgtatcttacaagtcagatgtatatagtttcggaatcttgttgcttgaaatgattgggggaaagaaaaattttaaagttatggaggactccaccagccatatctacttcccagaatggatttataaccttttagaacaagggagtgatctacgaatccatatcgaggacgaagaagatgttatattagctagaaaactagcaatcatcggtctatggtgcattcaatggcatccaatagatcgcccatccatgagcaatgttgttcaaatgttggagagagaaggtgacagtctaagcatgcctccttatccctttgcctctacttcaaattga

SEQ ID NO: 2 - protein, 611 aa, Fragaria sp.
MLLNLLCCWFLIGAVLDVDIIVHGESVGPENCTEVRCKRHGPVIRFPFRLKGMQPLHCGYPGFDLSCTTDNQTVLQINSSSADKFLVKRIDYTAQEIEIYLYNSHYDYFNDFQYICIPRQIFELSSSPFEFSDAQRKTNYTIFSCPPSDKRDRSPSFCPLAKLSPCLGNHSNRMYALQNGCAELKYMPQLVSCTKVEDYMSVPSVWMQPVKLKWTRPSCKRCEEMGKKCGFLNQVADDQKTEETQCLEVPKGPNRGKRILEILGICALATVLTGVATVIYYVYSSKKTEEENQLRIEKFLDDYRALKPSRYAYGDIRRITNQFEDKLGQGAYGTVYKGKLSSELLVAVKILDNSHEKGEDFINEVGTMGLIHHVNVVRLVGFCADGFIRALIYEYLPNGSLQNYLSSADDKNSFLGWEKLQDIALGIAKGIEYLHQGCDQRILHFDIKPSNILLDQEFTPKVSDFGLAKLCSKDQSAISMTTARGTMGYIAPEVFSRNFGNVSYKSDVYSFGILLLEMIGGKKNFKVMEDSTSHIYFPEWIYNLLEQGSDLRIHIEDEEDVILARKLAIIGLWCIQWHPIDRPSMSNVVQMLEREGDSLSMPPYPFASTSN

SEQ ID NO: 3 - DNA, 1899 bp, Fragaria sp.
  source 1..1899: mol_type = other DNA
atgtctagaagaagtctcctttttgcttcttacaccatcctcctcctccttccttttacagttatttcacaatcgtcggataccggaaatcctttagatatctgtacctcttcttgtggaggtatttacataagctatccatttcgactgaaaggagaccctcaaaactgtggaaacgaaaagtttgagctgtcttgcgaggcaaatagtactactgcagtactatacttggaatcagaagagtattatgtgaagtcgatccattacaataacttcacaatccgaatcgtggattccggtgttcagaagaagcggaactactgctccaccggactacactctatgaccaccttcgactctagtgatccatatagcttctcttccttgtacactccttacatggaacaagtgccaataatacttgtgacctgcgcaactccggtgaattccactctctttattgaaacagcttcatgcctcaacagcaccaccatcgattcttccaaatacaactatttcatggttggctctgtaacttcatttgatttgaagagttcttgtgaagtaacacaaatgcttttaacatccccttcaatcaacaacagcttcttgatgtcatgtgaaagcatatgtaatgaaatagtgaatggttttgaactctcatggtttacttatggatgtcaaaaatgcaaatctgatgaggtgtgcgacacgtcgtacgactctgacatcccaccggagctcaagactatcaagtgcagtaggcattcctcaggagaacgctggtatgacccgataaagaataagttttcagggaaacttctcatatataactctaaatttcatgttcagctccttctaaatgcacccgtttattacgcaatactacaagcagctaaacttgtaattggcattccttgtttaactgcattactgatatacaagtggaggagaagacatttgtcgatgtacgacgatatagaagacttcctgcggactaataataatctcatgccagtgagatacacttactcagacatcaagaagatgacaagggatttcaaggaaaaattgggtcaaggaggctatgggacggtatacaaggcaacccttcgtagtggtcggcttgtagccattaagatgttgggtaagtcccaagctaacgggcaagacttcatcaatgaagtcgcgaccattggaaggattcaccatgttaatgttgtgcaactaattggattttgtgttgagggatccaagcgtgctcttgtgtatgatttcatgcctaatgggtctcttgacaagtacatttttcttccacaaggagtgagcaccttaagtttcaagaaattttttgaaattgcgcttggagttgctcgtggtatcgactatctgcatcgagggtgtgatatgcaaattttgcactttgacatcaagcctcataacattcttctggacgagaatttcattcccaaggtttctgattttgggttagcaaggctatacccattggataatagcatcgtgtctctgactgctgtaagaggcacgataggatacatagctcctgagctattctataagaacattggaggtgtttcctacaaggctgatgtatatagttttggaatgctattgatggaaatggctgggagaaggaagaacttgaatgcagggatagaatattcaagccaatttagtgaaaattactttccttcatggcccagtgaacgcccttcaatgacaaaagtagtcgagatgcttgaaggagaactagtgagcctccaaatacccccaaagcctttcttatatccacaacaaatgccagtagaggatattggagacacttcttctactaccgatggatcgacaatgatggaagtgtcaacagagatcaactga

SEQ ID NO: 4 - protein, 632 aa, Fragaria sp.
MSRRSLLFASYTILLLLPFTVISQSSDTGNPLDICTSSCGGIYISYPFRLKGDPQNCGNEKFELSCEANSTTAVLYLESEEYYVKSIHYNNFTIRIVDSGVQKKRNYCSTGLHSMTTFDSSDPYSFSSLYTPYMEQVPIILVTCATPVNSTLFIETASCLNSTTIDSSKYNYFMVGSVTSFDLKSSCEVTQMLLTSPSINNSFLMSCESICNEIVNGFELSWFTYGCQKCKSDEVCDTSYDSDIPPELKTIKCSRHSSGERWYDPIKNKFSGKLLIYNSKFHVQLLLNAPVYYAILQAAKLVIGIPCLTALLIYKWRRRHLSMYDDIEDFLRTNNNLMPVRYTYSDIKKMTRDFKEKLGQGGYGTVYKATLRSGRLVAIKMLGKSQANGQDFINEVATIGRIHHVNVVQLIGFCVEGSKRALVYDFMPNGSLDKYIFLPQGVSTLSFKKFFEIALGVARGIDYLHRGCDMQILHFDIKPHNILLDENFIPKVSDFGLARLYPLDNSIVSLTAVRGTIGYIAPELFYKNIGGVSYKADVYSFGMLLMEMAGRRKNLNAGIEYSSQFSENYFPSWPSERPSMTKVVEMLEGELVSLQIPPKPFLYPQQMPVEDIGDTSSTTDGSTMMEVSTEIN

SEQ ID NO: 5 - DNA, 22 bp, synthetic construct
  source 1..22: mol_type = other DNA
gcaccctctgatgtgattctat

SEQ ID NO: 6 - DNA, 20 bp, synthetic construct
  source 1..20: mol_type = other DNA
tttgatgggccagtcatctc

SEQ ID NO: 7 - DNA, 20 bp, synthetic construct
  source 1..20: mol_type = other DNA
ccaagaggaatacagcaagg

SEQ ID NO: 8 - DNA, 22 bp, synthetic construct
  source 1..22: mol_type = other DNA
gttgtgatggcttgtaggagta

SEQ ID NO: 9 - DNA, 20 bp, synthetic construct
  source 1..20: mol_type = other DNA
gttgctcttgccaaggtttc

SEQ ID NO: 10 - DNA, 22 bp, synthetic construct
  source 1..22: mol_type = other DNA
caggagcaagagaatgagtatg

SEQ ID NO: 11 - DNA, 21 bp, synthetic construct
  source 1..21: mol_type = other DNA
caagctccacatcatcctatt

SEQ ID NO: 12 - DNA, 21 bp, synthetic construct
  source 1..21: mol_type = other DNA
ccatcgaatgtcttcctcttc

SEQ ID NO: 13 - DNA, 22 bp, synthetic construct
  source 1..22: mol_type = other DNA
ctaccacctaagctttccatac

SEQ ID NO: 14 - DNA, 21 bp, synthetic construct
  source 1..21: mol_type = other DNA
ggtggtagggcaataatactc

SEQ ID NO: 15 - DNA, 21 bp, synthetic construct
  source 1..21: mol_type = other DNA
cgggtttattcactgcttact

SEQ ID NO: 16 - DNA, 22 bp, synthetic construct
  source 1..22: mol_type = other DNA
gtactcgaggacaatgacatac

SEQ ID NO: 17 - DNA, 19 bp, synthetic construct
  source 1..19: mol_type = other DNA
cttctggctgatgtgttgt

SEQ ID NO: 18 - DNA, 21 bp, synthetic construct
  source 1..21: mol_type = other DNA
tgctcgtacgttactttactg

SEQ ID NO: 19 - DNA, 22 bp, synthetic construct
  source 1..22: mol_type = other DNA
ggtctcaagagagagacgtaaa

SEQ ID NO: 20 - DNA, 20 bp, synthetic construct
  source 1..20: mol_type = other DNA
tcctccaattgctcgtttag

SEQ ID NO: 21 - DNA, 20 bp, synthetic construct
  source 1..20: mol_type = other DNA
cctgaattcctggccatatc

SEQ ID NO: 22 - DNA, 22 bp, synthetic construct
  source 1..22: mol_type = other DNA
cctggccatatcaaaccaatag

SEQ ID NO: 23 - DNA, 18 bp, synthetic construct
  source 1..18: mol_type = other DNA
tctgcaccaccacatttc

SEQ ID NO: 24 - DNA, 22 bp, synthetic construct
  source 1..22: mol_type = other DNA
gtggcctcattaccctaataac

SEQ ID NO: 25 - DNA, 21 bp, synthetic construct
  source 1..21: mol_type = other DNA
aagaatctctcccactctctc

SEQ ID NO: 26 - DNA, 19 bp, synthetic construct
  source 1..19: mol_type = other DNA
gtaggccattgggaataag

SEQ ID NO: 27 - DNA, 20 bp, synthetic construct
  source 1..20: mol_type = other DNA
ctccttcccaaacccattaa

SEQ ID NO: 28 - DNA, 21 bp, synthetic construct
  source 1..21: mol_type = other DNA
ggtaatgaggccacacataaa

SEQ ID NO: 29 - DNA, 20 bp, synthetic construct
  source 1..20: mol_type = other DNA
atataatgcaaaaagtggct

SEQ ID NO: 30 - DNA, 20 bp, synthetic construct
  source 1..20: mol_type = other DNA
gtaatgacccttttctctcc

SEQ ID NO: 31 - DNA, 758 bp, synthetic construct
  source 1..758: mol_type = other DNA
ggggacaagtttgtacaaaaaagcaggctatgcagaaaatgcttctgaatttgttatgctgttggtttctcataggcgcagtgctagatgtagatattattgtgcatggagaatcagttggccctgaaaattgcacagaggttcgatgtaaacggcatggccctgtgatccgattcccatttcgactgaaaggaatgcaaccactccattgcggctacccgggctttgatctgtcatgcaccacggacaagcagactgtgctccagatcaactcatcatcagctgacaagttcttggttaaaaggattgactacactgctcaggaaattgaaatatatctttataatagtcactatgattatttcaatgatttccagtatatctgtattcctagacagattttcgagctcagttcctctccattcgaataatttcctgagcagtgtagtcaatccttttaaccaagaacttgtcagctgatgatgagttgatctggagcacagtctgcttgtccgtggtgcatgacagatcaaagcccgggtagccgcaatggagtggttgcattcctttcagtcgaaatgggaatcggatcacagggccatgccgtttacatcgaacctctgtgcaattttcagggccaactgattctccatgcacaataatatctacatctagcactgcgcctatgagaaaccaacagcataacaaattcagaagcattttctgcatacccagctttcttgtacaaagtggtcccc

SEQ ID NO: 32 - DNA, 758 bp, synthetic construct
  source 1..758: mol_type = other DNA
ggggacaagtttgtacaaaaaagcaggctatggcttgtatagagccacataacataaacaatctcccaaacatttctatttacccttatcatctctgtttcattacgccttcttcctgctcaccatgtctagaagaagtctcctttttgcttcttacagttatttcacaatcgtcggataccggaaatcctttagatatctgtacctcttcttgtggaggtatttacataagctatccatttcgactgaaaggagaccctcaaaactgtggaaacgaaaagtttgagctgtcttgcgaggcaaatagtactactgcagtactatacttggaatcagaagagtattatgtgaagtcgatccattacaataacttcacaatccgaatcgtggattccggtgttcagaagaagcggaactactgctccaccgcaagtatagtactgcagtagtactatttgcctcgcaagacagctcaaacttttcgtttccacagttttgagggtctcctttcagtcgaaatggatagcttatgtaaatacctccacaagaagaggtacagatatctaaaggatttccggtatccgacgattgtgaaataactgtaagaagcaaaaaggagacttcttctagacatggtgagcaggaagaaggcgtaatgaaacagagatgataagggtaaatagaaatgtttgggagattgtttatgttatgtggctctatacaagccatacccagctttcttgtacaaagtggtcccc

SEQ ID NO: 33 - DNA, 22 bp, synthetic construct
  source 1..22: mol_type = other DNA
cttctgaatttgttatgctgtt

SEQ ID NO: 34 - DNA, 19 bp, synthetic construct
  source 1..19: mol_type = other DNA
cgtttacatcgaacctctg

SEQ ID NO: 35 - DNA, 21 bp, synthetic construct
  source 1..21: mol_type = other DNA
gcttgtatagagccacataac

SEQ ID NO: 36 - DNA, 17 bp, synthetic construct
  source 1..17: mol_type = other DNA
catggtgagcaggaaga

SEQ ID NO: 37 - DNA, 26 bp, synthetic construct
  source 1..26: mol_type = other DNA
cccaagtaaggatgcccccatgttcg

SEQ ID NO: 38 - DNA, 28 bp, synthetic construct
  source 1..28: mol_type = other DNA
ttggcaaggggagcaagacagttggtag

SEQ ID NO: 39 - DNA, 20 bp, synthetic construct
  source 1..20: mol_type = other DNA
ttctcagacgcccaaagaaa

SEQ ID NO: 40 - DNA, 20 bp, synthetic construct
  source 1..20: mol_type = other DNA
agtggacagaaggatggaga

SEQ ID NO: 41 - DNA, 20 bp, synthetic construct
  source 1..20: mol_type = other DNA
cccgttagcttgggacttac

SEQ ID NO: 42 - DNA, 21 bp, synthetic construct
  source 1..21: mol_type = other DNA
aattgggtcaaggaggctatg

SEQ ID NO: 43 - protein, 609 aa, Fragaria vesca
MSRRSLLFASYSYFTIVGYRKSFRYLYLFLWRYLHKLSISTERRPSKLWKRKVAVLRGKYYCSTILGIRRVLCEVDPLQLHNPNRGFRCSEEAELLLHRTTLYGHLRLYSILLFLVHSLHGTSANNTCDLRNSGEFHSLYNSFMPQQHHHRFFQIQLFHGWLCNFIFEEFLSNTNAFNIPFNQQQLLDVMKHMNSEWFTLMVYLWMSKMQIGVRHVVRLHPTGAQDYQVQAFLRRTLVPDKEVFRETSHILISCSAPSKCTRLLRNTTSSTCNWHSLFNCITDIQVEEKTFVDVRRYRRLPADSHASEIHLLRHQEDDKGFQGKIGSRRLWDGIQGNPSWSACSHDVGVPSRARLYQSRDHWKDSPCCCATNWILCGIQACSCVFHAWVSQVHFSSTRSEHLKFQENFNCAWSCSWYRLSASRVYANFALHQASHSSGREFHSQGFFWVSKAIPIGHRVSDCCKRHDRIHSSAILEHWRCFLQGCIFWNAIDGNGWEKEELECRDRIFKPIKLLSFMGIPTERKRHRDRRCHGNENHKEDDCSSTVVHTNEAQTPFNDKSSRDARRTREPPNTPKAFLISTTNASRGYWRHFFYYRWIDNDGSVNRDQL

(19) United States
(12) Patent Application Publication
(10) Pub. No.: US 2026/0139266 A1
(43) Pub. Date: May 21, 2026

(54) GENES CONFERRING RESISTANCE TO ANTHRACNOSE FRUIT ROT OF STRAWBERRIES

(71) Applicant: University of Florida Research Foundation, Incorporated, Gainesville, FL (US)

(72) Inventors: Seonghee Lee, Lithia, FL (US); Vance M. Whitaker, Brandon, FL (US); Natalia Salinas, Marina, CA (US); Hyeondae Han, Riverview, FL (US)

(21) Appl. No.: 19/119,486

(22) PCT Filed: Oct. 27, 2023
(86) PCT No.: PCT/US2023/078004

§ 371 Date: Apr. 9, 2025

Related U.S. Application Data
(60) Provisional application No. 63/381,431, filed on Oct. 28, 2022.

(51) Int. Cl. C12N 15/82; C12N 5/04; C12N 5/10; C12N 9/22; C12N 15/113; C12Q 1/6844

(52) U.S. Cl. CPC C12N 15/8282; C12N 5/04; C12N 5/10; C12N 9/226; C12N 15/113; C12N 15/8213; C12Q 1/6844; C12N 2310/20; C12N 2510/00

(57) ABSTRACT

Described genetically modified Fragaria plants having increased resistance to anthracnose fruit rot. Compositions for modifying Fragaria plants to increase resistance to anthracnose fruit rot and methods of using the compositions to modify Fragaria plants are also described.

### CROSS-REFERENCE TO RELATED APPLICATIONS

This application claims the benefit of U.S. Provisional Application No. 63/381,431, filed Oct. 28, 2022, and is incorporated herein by reference.

### STATEMENT REGARDING FEDERALLY SPONSORED RESEARCH OR DEVELOPMENT

This invention was made with government support under Grant No. 2017-51181-26833 awarded by The United States Department of Agriculture, National Institutes of Food & Agriculture. The government has certain rights in the invention.

This invention was made whole or in part from funding received under contract received from the Florida Strawberry Research & Education Foundation

### SEQUENCE LISTING

The Sequence Listing written in file T18879W0001_SeqListing_ST26.xml is 45 kilobytes, was created on Sep. 28, 2023, and is hereby incorporated by reference.

### BACKGROUND

Cultivated strawberry (Fragaria×ananassa Duch.) is an allo-octoploid species (2n=8×=56) and originated by spontaneous interspecific hybrids of two wild octoploid species F. chiloensis Duch. and F. virginiana Duch (Duchesne A N, “Histoire naturelle des fraisiers, contenant les vues d'economie reunies a la botanique; & suivie de remarques particulieres sur plusieurs points qui ont rapport a l'histoire naturelle generale. Par m. Duchesne fils” (chez Didot le jeune, rue de Hurepoix, 1766)). It is believed that the octoploid progenitors of cultivated strawberry were generated by successive stages of polyploidization of four diploid progenitor species. The major diploid progenitor species were identified as F. vesca and F. iinumae. Because of their complex genome structures, breeding and genetics for octoploid strawberry have been neglected compared to diploid strawberries.

De nova genome assembly is the most ideal and comprehensive method providing unbiased information to DNA sequences. The first chromosome-scale genome assembly for octoploid strawberry ‘Camarosa’ (Edger P P et al. “Origin and evolution of the octoploid strawberry genome.” Nature genetics 51, 541-547 (2019)) has been performed based on combining Illumina Solexa's short reads (<1 kb) with a low rate of error (<0.1%) and Pacific Biosciences (PacBio) long reads with a high rate of error (<10%). The availability of the reference sequences contributed not only to studying evolutionary history of octoploid strawberry genome, but also to characterization of agronomical traits such as disease resistance and fruit quality (Barbey C R et al. “Genetic Analysis of Methyl Anthranilate. Mesifurane, Linalool, and Other Flavor Compounds in Cultivated Strawberry (Fragaria×ananassa)”. Frontiers in Plant Science, 718 (2021)). The first available octoploid strawberry genome of ‘Camarosa’ greatly helped to understand the evolution of chromosome and the subsequent origin of ancestral diploid progenitors. However, the draft genome v1 has limitations for wider use of strawberry genomics research and breeding, because of absence of haplotype-phased information, phase-switching, assembly gaps, scaffolding errors, and local assembly errors due to a short-read assembly of a heterozygous octoploid. It is important to have a high-quality haplotype-phased genome that resolves heterogeneous genome complexity and can be used to identify homoeologous variations at the subgenome level.

A great challenge for haplotype-phased genome assembly is base-calling accuracy. Recently, the availability of high-fidelity (HiFi) reads developed by PacBio has become the foundation for haplotype-phased genome assembly. The first haplotype-phased genome assembly for octoploid strawberry cv. ‘Royal Royce’ was assembled by applying trio-binning requiring parental sequencing data (Hardigan M A et al. “Blueprint for phasing and assembling the genomes of heterozygous polyploids: application to the octoploid genome of strawberry.” BioRxiv (2021)). However, the requirement of parental information often limits the haplotype-phased genome assembly in practice. Recently, a new algorithm combining PacBio HiFi reads and Hi-C chromatin interaction data (Cheng H et al. “Robust haplotype-resolved assembly of diploid individuals without parental data.” arXiv preprint arXiv:2109.04785 (2021)) generated fully haplotype-phased genome assembly in human genome study without parental data (Porubsky D et al. “Fully phased human genome assembly without parental data using single-cell strand sequencing and long reads.” Nature biotechnology 39. 302-308 (2021)).

### SUMMARY

Described are compositions and methods for generating genetically modified anthracnose fruit rot (AFR) resistant and/or anthracnose root necrosis (ARN) resistant Fragaria plants. Also described are markers for use in selecting AFR and ARN resistant Fragaria plants. The Tragaria plant can be, but is not limited to, an octoploid Fragaria plant. The modified plants can be used to introgress AFR or ARN resistance into other strawberry plant genetic backgrounds.

In some embodiments. genetically modifiedIFragaria (strawberry) plants modified at the FaRCa1 locus of the plant genome are described. The FaRCa1 locus of the Fragaria plant can be genetically modified to contain an insertion of a heterologous 163.0 (Receptor_kinasel) gene, coding sequence, or fragment thereof, and/or a heterologous 163.26 (Receptor_kinase2) gene, coding sequence. or a fragment thereof. In some embodiments, the heterologous 163.0 coding sequence comprises SEQ ID NO: 1 or encodes a protein having the amino acid sequence of SEQ ID NO: 2. In some embodiments, the heterologous 163.26 coding sequence comprises SEQ ID NO: 3 or encodes a protein having the amino acid sequence of SEQ ID NO: 4. The FaRCa1 locus of the Frigaria plant can be genetically modified to contain an insertion of a heterologous sequence encoding a 163.0 protein (SEQ ID NO: 2) and/or a heterologous sequence encoding a 163.26 protein (SEQ ID NO: 4) The genetically modified plants have increased resistance to Anthracnose fruit rot (AFR) and/or anthracnose root necrosis (ARN) compared to a control Fragaria plant the does not express the heterologous 163.0 gene, coding sequence, or fragment thereof and/or the heterologous 163.26 gene, coding sequence, or fragment thereof. In some embodiments, the Fragaria plant is an octoploid Fragaria plant.

In some embodiments, genetically modified Fragaria (strawberry) plants expressing a heterologous 163.0 gene, coding sequence, or fragment thereof and/or a heterologous 163.26 gene. coding sequence, or a fragment thereof, are described. The 163.0 and/or 163.26 gene, coding sequence, or fragment thereof can be inserted at any available locus in the Fragaria plant. An available locus can be any locus in the plant that allows for insertion and expression of a heterologous nucleic acid without substantially affecting growth or fruit production of the plant. The Fragaria plant can be genetically modified to contain an insertion of: a heterologous 163.0 gene, coding sequence, or fragment thereof, and/or a heterologous 163.26 gene, coding sequence, or a fragment thereof. In some embodiments, the heterologous 163.0 coding sequence comprises SEQ ID NO: 1 or encodes a protein having the amino acid sequence of SEQ ID NO: 2. In some embodiments, the heterologous 163.26 coding sequence comprises SEQ ID NO: 3 or encodes a protein having the amino acid sequence of SEQ ID NO: 4, The Fragaria plant can be genetically modified to contain an insertion of: a heterologous sequence encoding a 163.0 protein (SEQ ID NO: 2) and/or a heterologous sequence encoding a 163.26 protein (SEQ ID NO: 4). The genetically modified plants have increased resistance to Anthracnose fruit rot (AFR) and/or anthracnose root necrosis (ARN) compared to a control Fragaria plant the does not express the heterologous 163.0 gene, coding sequence, or fragment thereof and/or the heterologous 163.26 gene, coding sequence, or fragment thereof. In some embodiments, the Fragaria plant is an octoploid Fragaria plant.

The FaRCa1 or other available locus of the Fragaria plant can be genetically modified using methods available in the art for introducing a heterologous sequence into a plant. Such methods include, but are not limited to: unaided homologous recombination, recombinase-based insertion, and DNA repair-based insertion. DNA repair-based insertion can be, but is not limited to, nuclease agent-mediated DNA repair-based insertion. The nuclease agent can be, but is not limited to, a zinc finger nuclease (ZFN), a Transcription Activator-Like Effector Nuclease (TALEN). a meganuclease, a Clustered Regularly Interspaced Short Palindromic Repeats (CRISPR)-associated (Cas) protein and a guide RNA (gRNA). In some embodiments, the nuclease agent comprises a Cas protein and a gRNA. A gRNA comprises a CRISPR RNA (crRNA) and a trans-activating CRISPR RNA (tracrRNA), as separate molecules or as a single chimeric guide RNA (sgRNA).

Introducing a heterologous 163.0 gene, coding sequence, or fragment thereof and/or a heterologous 163.26 gene or a fragment thereof into a Fragaria plant, plant tissue, or plant cell can be done using methods available in the art for introducing a heterologous nucleic acid sequence into a plant or plant cell. The Such methods include, but are not limited to: electroporation, microprojectile bombardment. biolistic transformation. microinjection. protoplast transformation, Agrobacterium tumefaciens vector transformation and Agrobacterium rhizogenes vector transformation. The heterologous 163.0 gene, coding sequence, or fragment thereof and/or a heterologous 163.26 gene or a fragment thereof can be introduced at the FaRCa1 locus or at another position in the plant genome.

Following introduction of a heterologous 163.0 gene, coding sequence, or fragment thereof and/or a heterologous 163.26 gene or a fragment thereof into a Fragaria plant, plant tissue, or plant cell one or more regenerants can be generated. The regenerants can be genotyped for the presence of the heterologous 163.0 gene, coding sequence. or fragment thereof and/or the heterologous 163.26 gene or a fragment thereof. In some embodiments, one or more To plants containing one or more of the heterologous 163.0 gene, coding sequence, or fragment thereof and/or the heterologous 163.26 gene, coding sequence or a fragment thereof are selected.

In some embodiments, methods of genetically modifying a Fragaria plant at the FaRCa1 locus to produce a plant resistant to AFR and/or ARN are described. The methods comprise introducing a Cas protein or a nucleic acid encoding the Cas protein and a gRNA or a nucleic acid encoding the gRNA into a Fragaria plant cell, wherein the gRNA and Cas protein form a complex that targets the FaRCa1 locus to insert one or more of a heterologous 163.0 gene, coding sequence, or a fragment thereof and a 163.26 gene, coding sequence, or a fragment thereof, and generating a regenerant plant from the Fragaria plant cell, wherein the regenerant plant expresses the heterologous 163.0 and/or 163.26 gene. coding sequence, or a fragment thereof. In some embodiments, the Fragaria plant is an octoploid Fragaria plant.

In some embodiments, methods of genetically modifying a Fragaria plant to produce a plant resistant to AFR and/or ARN are described. The methods comprise introducing a Cas protein or a nucleic acid encoding the Cas protein and a gRNA or a nucleic acid encoding the gRNA into a Fragaria plant cell, wherein the gRNA and Cas protein form a complex that targets an available locus to insert one or more of a heterologous 163.0 gene, coding sequence, or a fragment thereof and a 163.26 gene, coding sequence, or a fragment thereof, and generating a regenerant plant from the Fragaria plant cell, wherein the regenerant plant expresses the heterologous 163.0 and/or 163.26 gene, coding sequence, or a fragment thereof. In some embodiments, the Fragaria plant is an octoploid Fragaria plant.

In some embodiments, methods of genetically modifying a Fragaria plant are described comprising: introducing a CRISPR system into a Fragaria plant cell, wherein the CRISPR system comprises: (a) an RNA-guided DNA endonuclease or a nucleic acid encoding the RNA-guided DNA endonuclease; (b) a guide RNA or a nucleic acid encoding the guide RNA; and (c) a DNA donor template containing a nucleic acid sequence encoding a heterologous 163.0 gene sequence, coding sequence, or a fragment thereof and/or a DNA donor template containing a nucleic acid sequence encoding a heterologous 163.26 gene sequence, coding sequence, or a fragment thereof; wherein the RNA-guided DNA endonuclease and the guide RNA form a complex that creates a single or double strand break in the genome of the Fragaria plant cell and the DNA donor template is used by the plant cell to repair the break and insert the nucleic acid sequence encoding the 163.0 gene, coding sequence or the fragment thereof and/or the 163.26 gene, coding sequence. or the fragment thereof into the genome at the site of the single or double strand break. The CRISPR system can be introduced into the Fragaria plant cell using methods available in the art for introducing a CRISPR system into a plant cell. Such methods include, but are not limited to: electroporation, microprojectile bombardment, biolistic transformation. microinjection, protoplast transformation, Agrobacterium tumefaciens vector transformation, and Agrobacterium rhizogenes vector transformation. In some embodiments, the DNA donor template comprises SEQ ID NO: 1 or a fragment thereof or a complement thereof. In some embodiments, the DNA donor template comprises a sequence encoding SEQ ID NO: 2 or a fragment thereof. In some embodiments, the DNA donor template comprises SEQ ID NO: 3 or a fragment thereof or a complement thereof. In some embodiments, the DNA donor template comprises a sequence encoding SEQ ID NO: 4 or a fragment thereof. In some embodiments, the CRISPR system targets the FaRCa1 locus. In some embodiments, the genetically modified Fragaria plant cell overexpresses the 163.0 gene, coding sequence, or a fragment thereof and/or the 163.26 gene, coding sequence, or a fragment thereof. In some embodiments, the Fragaria plant is an octoploid Fragaria plant.

Following introduction of the heterologous 163.0 gene, coding sequence, or fragment thereof and/or the heterologous 163.26 gene, coding sequence, or a fragment thereof into the Fragaria plant cell, one or more regenerants can be generated. The regenerants can be genotyped for the presence of the heterologous 163.0 gene, coding sequence, or fragment thereof and/or the heterologous 163.26 gene, coding sequence, or a fragment thereof. In some embodiments, one or more To plants containing one or more of the heterologous 163.0 gene, coding sequence, or fragment thereof and/or the heterologous 163.26 gene, coding sequence, or fragment thereof are selected. In some embodiments, the regenerant or the To plant has increased resistance to AFR and/or ARN.

Also described are methods of introgressing AFR and/or ARN resistance into a Fragaria plant. The methods comprise crossing at least one donor Fragaria plant known to express or overexpress a 163.0 protein and/or a 163.26 protein with at least one recipient Fragaria plant in order to form a segregating population; and screening the segregating population with one or more FaRCa1 markers to determine if one or more Fragaria plants from said segregating population contains or expresses the 163.0 protein and/or a 163.26 protein, wherein the one or more FaRCa1 markers comprise one or more detectable genetic markers linked, closely linked, tightly linked, or extremely tightly linked to the region of chromosome Fvb6-3 between base pair 16,238,392 and base pair 16,403,945. The FaRCa1 marker can be, but is not limited to, a PCR amplification product, a single nucleotide polymorphism (SNP), a restriction fragment length polymorphism (RFLP), an amplified fragment length polymorphism (AFLP), a simple sequence repeat (SSR), a simple sequence length polymorphism (SSLP), an insertion/deletion polymorphism (indel), a variable number tandem repeat (VNTRs), or a random amplified polymorphic DNA (RAPD). In some embodiments, the PCR amplification product comprises all or a portion of SEQ ID NO: 1 or SEQ ID NO: 3. In some embodiments, the FaRCa1 marker is in a continuous nucleic acid region comprising an interval between 16,238,392 and 16,403,945 bp on chromosome 6-3 of the Fragaria plant. In some embodiments, screening the segregating population comprises amplifying a nucleotide sequence containing the FaRCa1 marker to produce an amplification product, wherein the amplification product is determinative for the presence and/or absence of the FaRCa1 marker. In some embodiments, the Fragaria plant is an octoploid Fragaria plant.

In some embodiments, introgressing AFR and/or ARN resistance into a Fragaria plant comprises crossing at least one donor Fragaria plant known to contain and express a heterologous 163.0 gene, coding sequence, or fragment thereof, and/or a heterologous 163.26 gene, coding sequence, or fragment thereof, with at least one recipient Fragaria plant that does not express the heterologous 163.0 gene, coding sequence, or fragment thereof, and/or a heterologous 163.26 gene, coding sequence, or fragment thereof in order to form a segregating population; and screening the segregating population with one or more markers to determine if one or more Fragaria plants from said segregating population contains or expresses the heterologous 163.0 gene, coding sequence, or fragment thereof, and/or a heterologous 163.26 gene, coding sequence, or fragment thereof. The marker can be, but is not limited to, a PCR amplification product, a single nucleotide polymorphism (SNP), a restriction fragment length polymorphism (RFLP), an amplified fragment length polymorphism (AFLP), a simple sequence repeat (SSR), a simple sequence length polymorphism (SSLP), an insertion/deletion polymorphism (indel), a variable number tandem repeat (VNTRs), or a random amplified polymorphic DNA (RAPD). In some embodiments, the PCR amplification product comprises all or a portion of SEQ ID NO: 1 or SEQ ID NO: 3. In some embodiments, screening the segregating population comprises amplifying a nucleotide sequence containing the marker to produce an amplification product, wherein the amplification product is determinative for the presence and/or absence of the 163.0 gene, coding sequence, or fragment thereof, and/or a heterologous 163.26 gene. coding sequence, or fragment thereof. In some embodiments, the Fragaria plant is an octoploid Fragaria plant.

### BRIEF DESCRIPTION OF THE DRAWINGS

FIG. 1. Smudgeplot for octoploid strawberry ‘Florida Brilliance’. The brightness of each smudge is determined by the number of k-mer pairs, which calculated by jellyfish. The coloration indicates the approximate number of k-mer pairs per bin. Brightest smudge ‘AAAAAABB’ (0.36) suggests ‘Florida Brilliance’ is allo-octoploid with closely related paralogs. Other smudges including ‘AAAABB’ (0.28), ‘AABB’ (0.25) reflect that A and B loci are more closely related.

FIG. 2. Hi-C contact map of phased-1 (A) and phased-2 (B) genome assemblies of ‘Florida Brilliance’. Each dark pixel represents the Hi-C pairs. The dominant visual feature in every Hi-C heat map is the strong diagonal representing the Hi-C pairs of loci uniquely anchored in each haplotype-phased assembly.

FIG. 3. Dotplot of ‘Florida Brilliance’ phased-2 assembly to ‘Florida Brilliance’ phased-1 assembly (A), diploid F. vesca ver 4.0 (B), F.×ananassa cv. Royal Royce (C), and F. Chiloensis (D). Dot plots are produced using the DGENIE software and alignments with minimap2.

FIG. 4A. Identification of rust resistance Lr10-like genes 163.0 (FxaC_22g29230) and 163.26 (FxaC 22g29220) in chromosome Fvb6-3 of the octoploid ‘Camarosa’ reference genome v1.0.a2. A) FaRCa1 region. Vertical dotted lines (with headers (AX-# or 9=bp InDel) indicate position of IStraw 35 Affymterix Axiom® SNPs and 50K array SNPs. A screenshot of the genes in the 165.63 kb region was obtained from JBrowse 1.16.4 using the Fragaria×ananassa v1.a2 annotation. Position of overlapping resistant and susceptible alleles obtained from BAC library sequencing is shown in gray boxes. “SL01+SL02” and “SL03+SL04” respectively. Box beneath 16,300,000 indicates genes 163.0 (FxaC_22g29230) and 163.26 (FxaC_22g29220).

FIG. 4B Gene structure of resistant alleles of genes 163.0 (FxaC 22g29230) and 163.26 (FxaC_22g29220). Gray rectangles and black lines represent exons and introns, respectively. Green box indicates insertions. Green lines represent the position of SNPs between susceptible allele in ‘Camarosa’ and resistant allele in ‘Florida Brilliance’ BAC clones (base in ‘Camarosa’>base in ‘Florida Brilliance.’

FIG. 4C. Amino acid sequence of resistant allele (R; SEQ ID NO: 4) and susceptible (S, SEQ ID NO: 43) allele for 163.26 (FxaC_22g29220). Protein kinase domain is highlighted in according to InterPro 87.0.

(SEQ ID NO: 43)
MSRRSLLFASYSYFTIVGYRKSFRYLYLFLWRYLHKLSISTERRP
SKLWKRKVAVLRGKYYCSTILGIRRVLCEVDPLQLHNPNRGFRCS
EEAELLLHRTTLYGHLRLYSILLFLVHSLHGTSANNTCDLRNSGE
FHSLYNSFMPQQHHHRFFQIQLFHGWLCNFIFEEFLSNTNAFNIP
ENQQQLLDVMKHMNSEWFTLMVYLWMSKMQIGVRHVVRLHPTGAQ
DYQVQAFLRRTLVPDKEVFRETSHILISCSAPSKCTRLLRNTTSS
TCNWHSLFNCITDIQVEEKTFVDVRRYRRLPADSHASEIHLLRHQ
EDDKGFQGKIGSRRLWDGIQGNPSWSACSHDVGVPSRARLYQSRD
HWKDSPCCCATNWILCGIQACSCVFHAWVSQVHFSSTRSEHLKFQ
ENFNCAWSCSWYRLSASRVYANFALHQASHSSGREFHSQGFFWVS
KAIPIGHRVSDCCKRHDRIHSSAILEHWRCFLQGCIFWNAIDGNG
WEKEELECRDRIFKPIKLLSFMGIPTERKRHRDRRCHGNENHKED
DCSSTVVHTNEAQTPFNDKSSRDARRTREPPNTPKAFLISTTNAS
RGYWRHFFYYRWIDNDGSVNRDQL

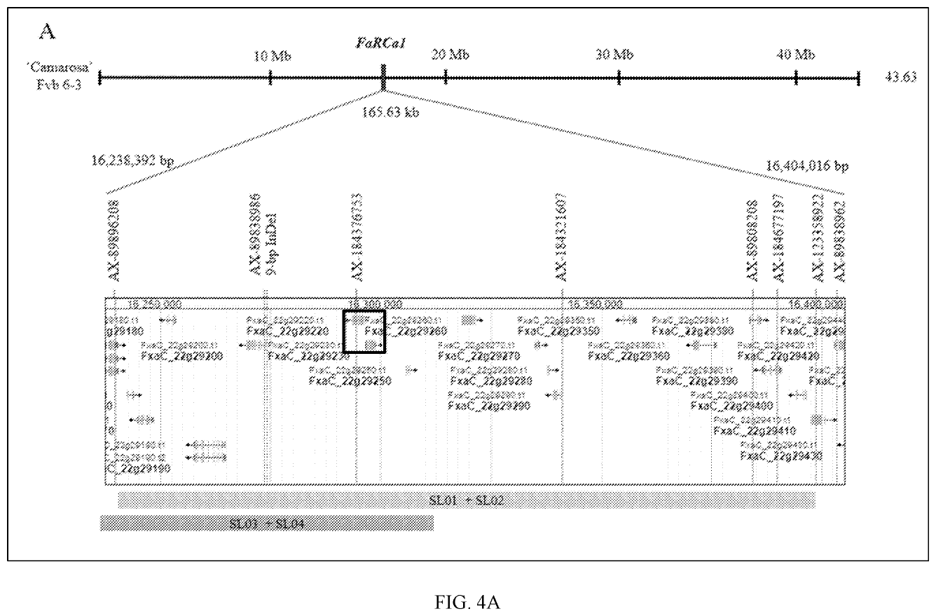

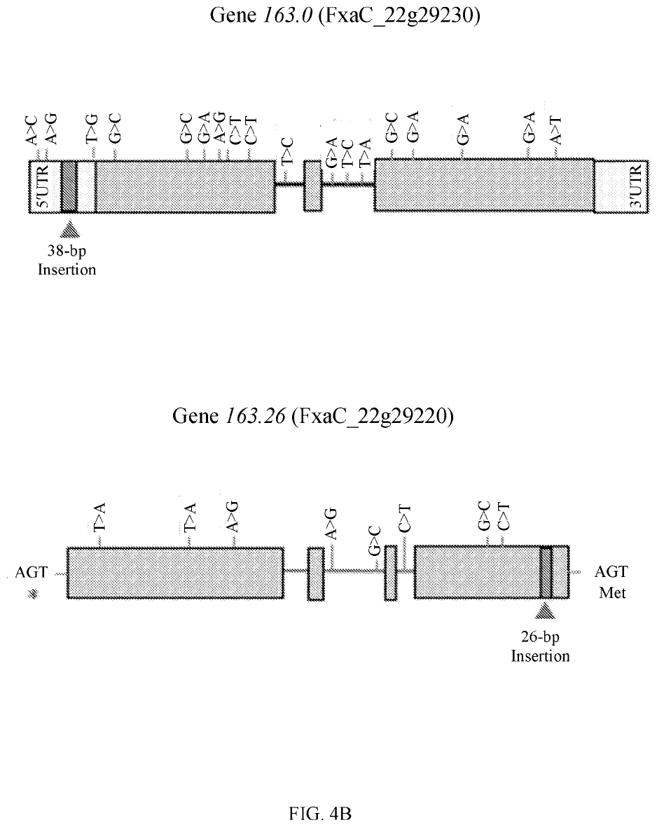

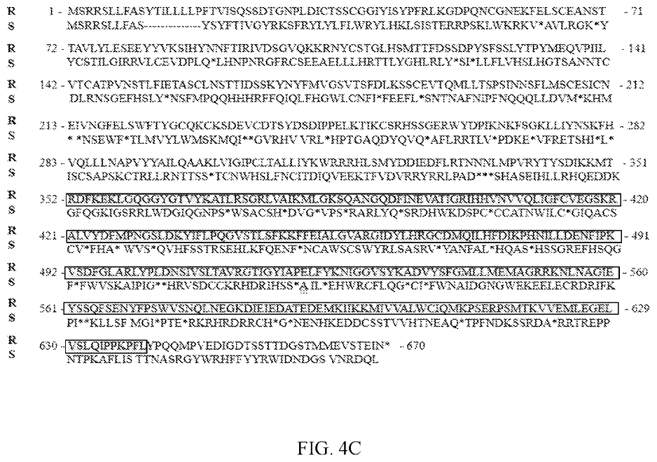

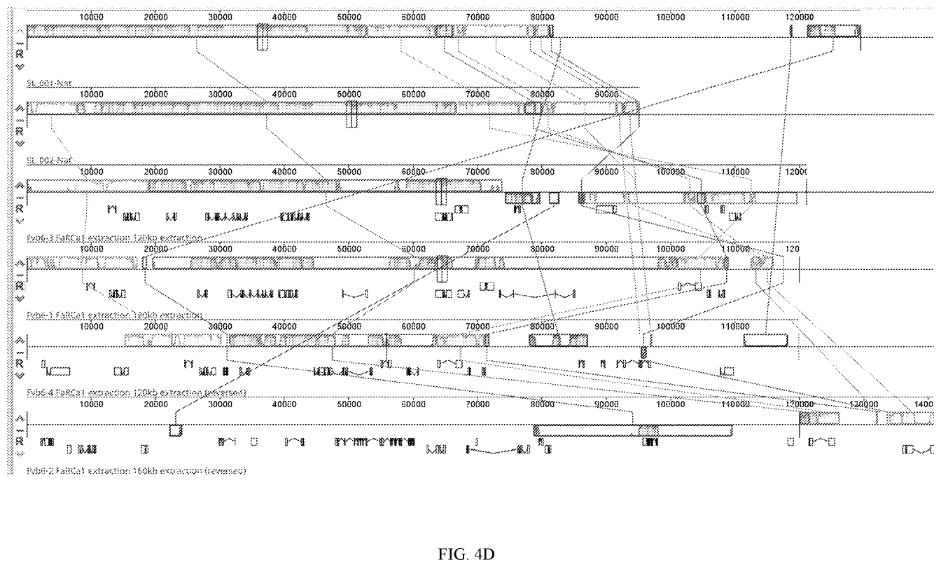

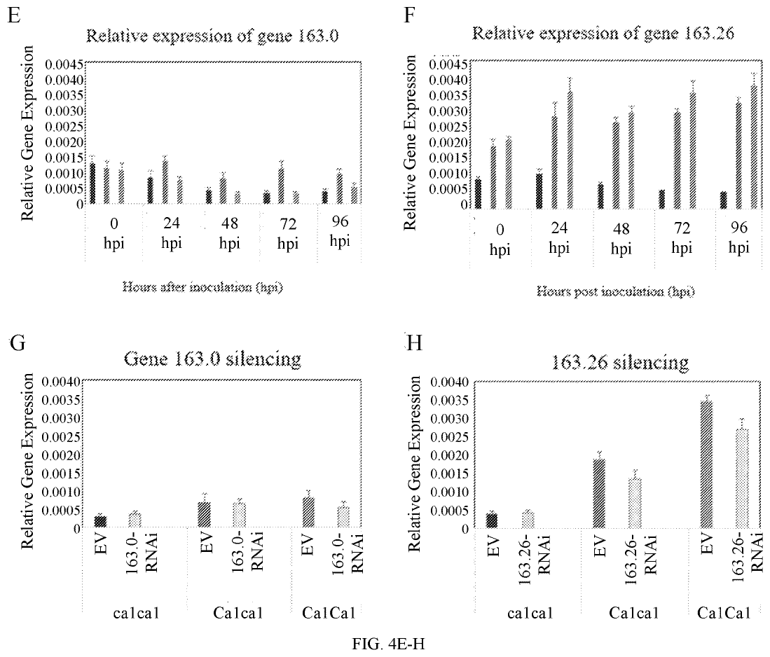

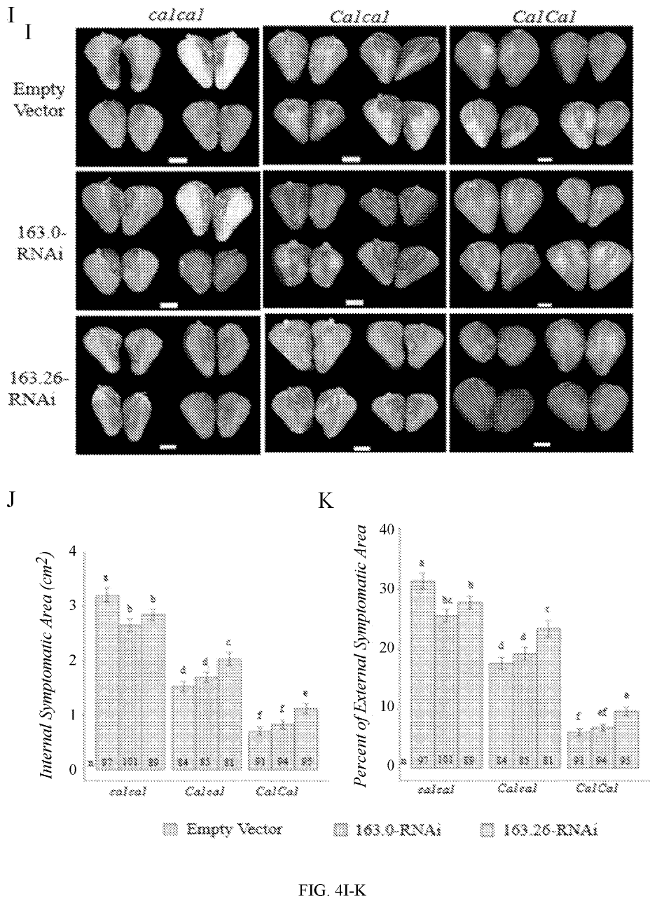

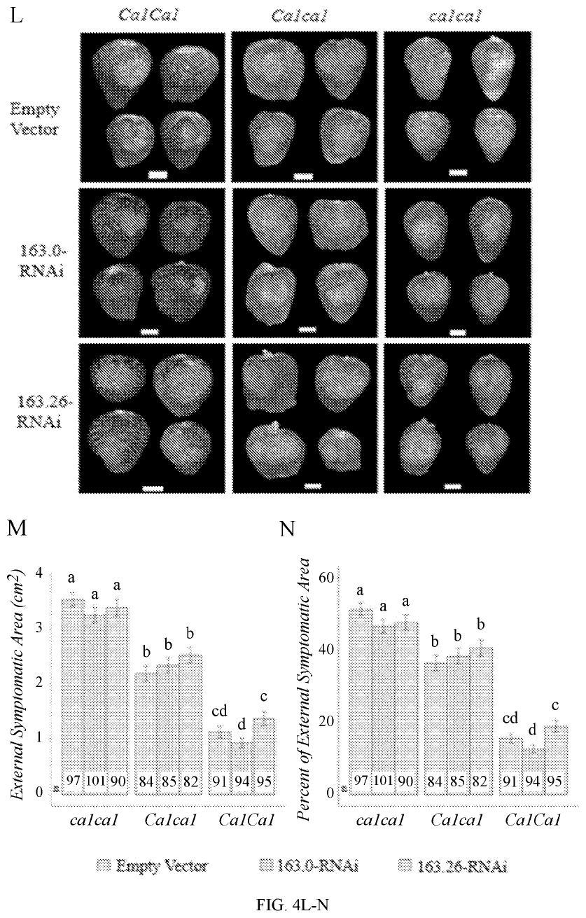

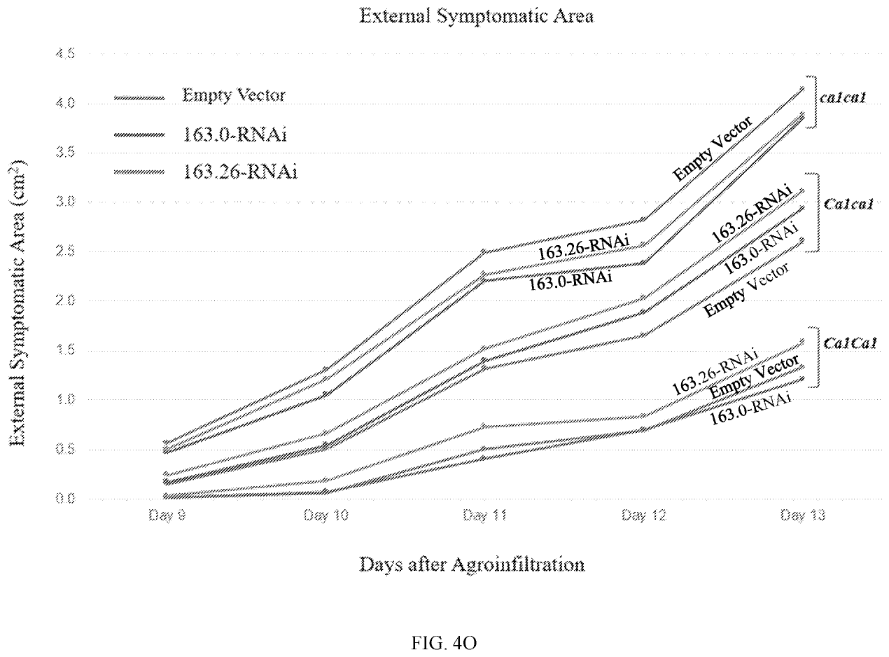

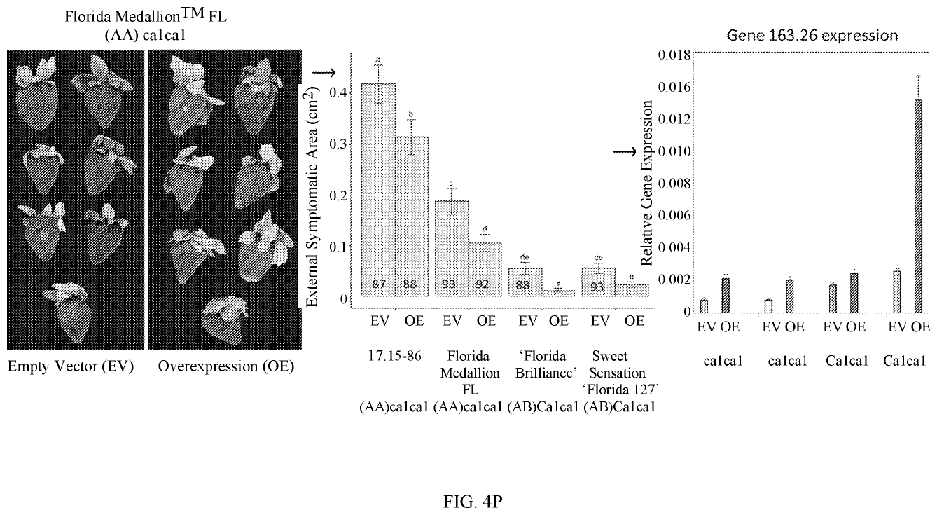

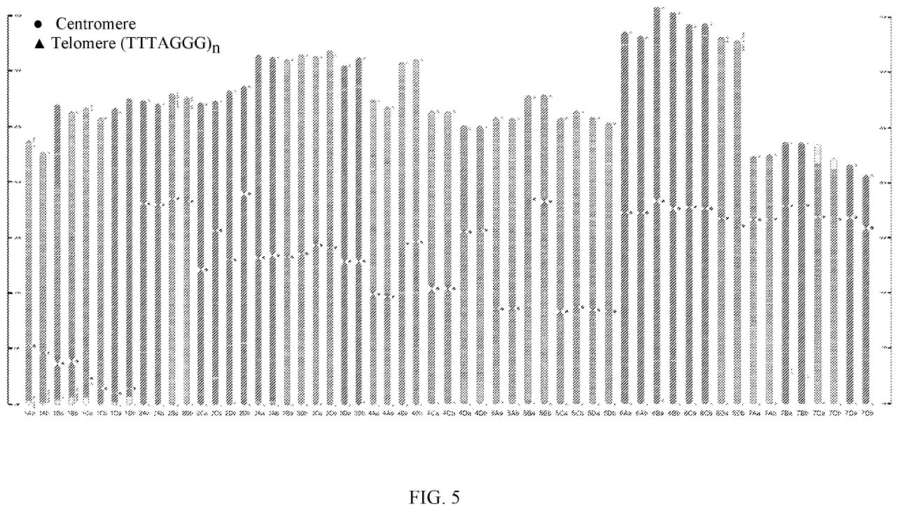

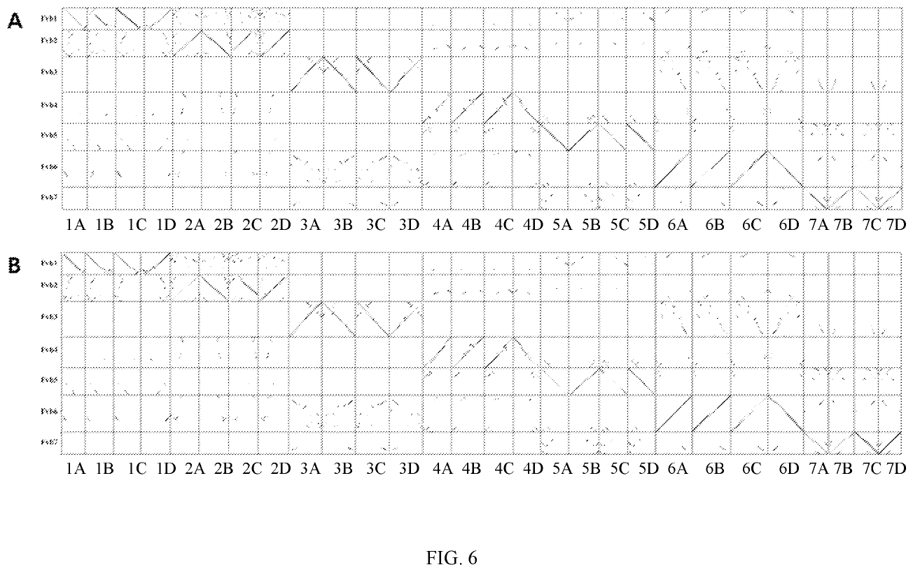

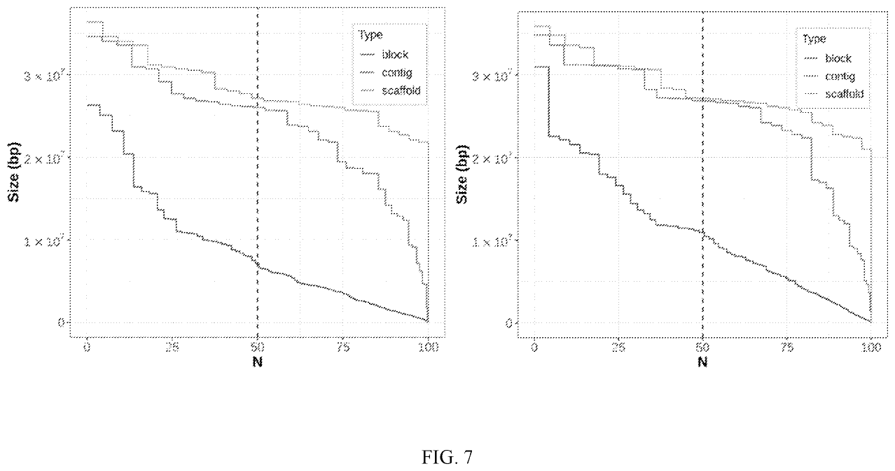

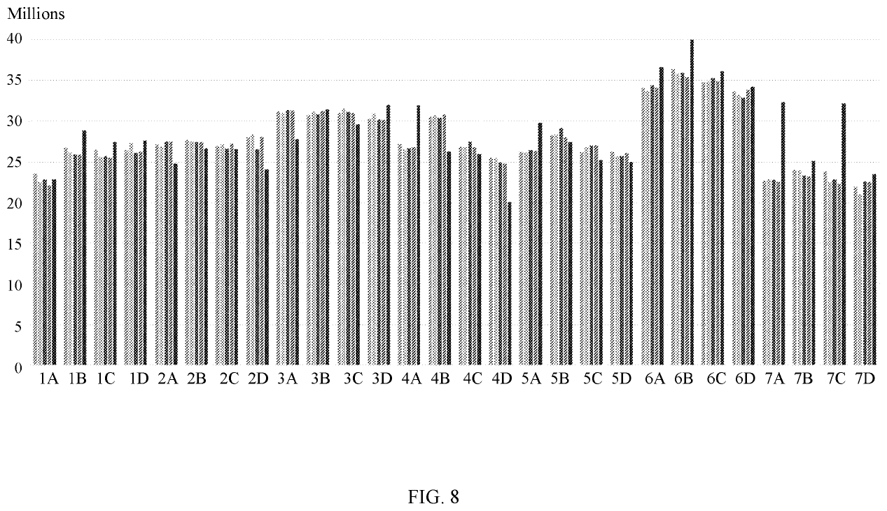

FIG. 4D. Comparative genomics analysis using Mauve alignment152 in Geneious Prime® 2022.0.1 with resistant allele from ‘Florida Brilliance’ BAC clone and regions homologous to FaRCa1 in subgenomes Fvb 6-1, 6-2, 6-3 and 6-4 from ‘Camarosa’ genome. Black vertical rectangle shows the position of gene 163.26 in the different sequences. Resistant alignments, SL000 and SL002, showed ten locally collinear blocks (LCB) when aligned with homologous FaRCa1 regions in Fvb 6-1, 6-2, 6-3 and 6-4.

FIG. 4E-H. Relative gene expression for E) Gene 163.0 and F) Gene 163.26 after C. acutatum inoculation for genotypes ca1ca1, Ca1ca1, and Ca1Ca1 (respectively first, second, and third bars for each hpi series), and for G) Gene 163.0 and H) Gene 163.26 in nonsilenced (empty vector (EV). bark bars) and silenced fruit (163.0:RNAi and 163.26:RNAi. light bars) for genotypes ca1ca1, Ca1ca1 and Ca1Ca1. FaGAPDH2 was used as internal gene control. Bars in E and F represent mean±standard deviation (SD) of ten biological replicates with 4 technical replicates. Bars in G and H represent mean±standard deviation (SD) of three biological replicates with three technical replicates.

FIG. 4I-K. Internal symptoms of anthracnose fruit rot in fruit from three genotypes: ca1 ca1, Ca1ca1 and Ca1Ca1, agroinfiltrated with three treatments: Empty Vector, 163.0:RNAi, and 163.26:RNAi. I) Phenotype of agroinfiltrated fruit. White line represents scale of 1 cm, J) Internal symptomatic area (cm2) and K) Percentage of internal symptomatic area. Lowercase letters indicate significantly different means using least significant difference test (α=0.05). Bars indicate standard errors.

FIG. 4L-N. External symptoms of anthracnose fruit rot in fruit from three genotypes: ca1ca1. Ca1ca1 and Ca1Ca1, agroinfiltrated with three treatments: Empty Vector (first bar in each series), 163.0:RNAi (second bar in each series), and 163.26:RNAi (third bard in each series). L) Phenotype of agroinfiltrated fruit. White line represents scale of 1 cm, M) External symptomatic area (cm2) and N) Percentage of external symptomatic area. Lowercase letters indicate significantly different means using least significant difference test (α=0.05). Bars indicate standard errors.

FIG. 4O. Development of external symptomatic area at Day 9, 10, 11, 12 and 13 after agroinfiltration for three genotypes: ca1ca1, Ca1ca1 and Ca1Ca1, agroinfiltrated with three treatments: Empty Vector, 163.0:RNAi, and 163.26:RNAi.

FIG. 4P. External symptoms of anthracnose fruit rot in fruit from three genotypes: ca1ca1 (susceptible) and Ca1ca1 (resistance) agroinfiltrated with three treatments: Empty Vector (EV) and 163.26-Overexpression (OE). Phenotype of agroinfiltrated fruit in ‘Florida Medallion’ (left panel).

FIG. 5. Telomere-to-telomere haplotype-phased genome assembly for octoploid strawberry ‘Florida Brilliance’. Triangle (Orange) represent the telomere sequences (5′-TTTAGGG-3′). Multiple triangles represent interstitial telomere-like sequence. Circles (purple) indicate the centromere region with low gene density and high density of repetitive sequences including LTR RTs and mini-satellites.

FIG. 6. Dotplot of collinear gene pairs between ‘Florida Brilliance’ and F. vesca. Homologous genes of phased-1 (A) and phased-2 (B) assemblies were compared against diploid F. vesca.

FIG. 7. N50 of phased block, contig, and scaffolds in phased-1 (A) and phased-2 assembly (B).

FIG. 8. Chromosome length of ‘Florida Brilliance’, ‘Royal Royce’ and ‘Camarosa’ assemblies. ‘Florida Brilliance’ and ‘Royal Royce’ consist of two phased assemblies (phased-1 and phased-2). For each series, bars represent, in order, FaFB1_hap1, FaFB1_hap2, FaRR hap1, FaRR hap2, and Camarosa.

### DETAILED DESCRIPTION

Unless otherwise defined, all terms of art, notations, and other scientific terminology used herein are intended to have the meanings commonly understood by those of skill in the art to which this invention pertains. Some techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y.; Current Protocols in Molecular Biology (Ausbel et al., eds., John Wiley & Sons, Inc. 2001; Transgenic Plants: Methods and Protocols (Leandro Pena, ed., Humana Press, 1st edition, 2004); and, Agrobacterium Protocols (Wan, ed., Humana Press, 2nd edition, 2006). As appropriate, procedures involving the use of commercially available kits and reagents are generally carried out in accordance with manufacturer defined protocols and/or parameters unless otherwise noted.

The use of “comprises,” “comprising,” “contain,” “contains,” “containing,” “include,” “includes.” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

The term “about” or “approximately” indicates within an acceptable error range for the particular value as determined by one of ordinary skill in the art, which will depend in part on how the value is measured or determined, e.g., the limitations of the measurement system. For example. “about” can mean within 1 or more than 1 standard deviation, per the practice in the art. Alternatively, “about” can mean a range of 0 to 20%, 0 to 10%, 0 to 5%, or up to 1% of a given value. Where particular values are described in the application and claims, unless otherwise stated the term “about” means within an acceptable error range for the particular value should be assumed.

All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as well as whole numbers in between the end values, and where practical. rational numbers within the range (e.g., the range 5-10 includes 5, 6, 7. 8, 9, and 10, and where practical, values such as 6.8, 9.35. etc.). When values are expressed as approximations, by use of the antecedent “about,” it will be understood that the particular value forms a further aspect. For example, if the value “about 10” is disclosed, then “10” is also disclosed.

The term “nucleic acid” refers to deoxyribonucleotides or ribonucleotides and polymers thereof (“polynucleotides”) in either single- or double-stranded form. Unless specifically limited, the term polynucleotide encompasses nucleic acids containing known analogues of natural nucleotides which have similar binding properties as the reference nucleic acid and are metabolized in a manner similar to naturally occurring nucleotides. Unless specifically limited, the term polynucleotide encompasses nucleic acids having one or more modified nucleotides. Modified nucleotides can modify binding properties or alter in vitro or in vivo stability. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxvinosine residues (Batzer et al., 1991. Nucleic Acid Res. 19: 5081; Ohtsuka et al., 1985 J. Biol. Chem. 260: 2605-2608; and Cassol et al., 1992; Rossolini et al., 1994, Mol. Cell. Probes 8: 91-98). The term nucleic acid is used interchangeably with gene, cDNA, and mRNA encoded by a gene.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted, and other elements added without sacrificing the necessary expression.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window. or designated region as measured using a sequence comparison algorithms, or by manual alignment and visual inspection.

Sequence identity can be determined by aligning sequences using algorithms. such as BESTFIT. FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. The term “isolated” may include proteins and nucleic acids that have no naturally occurring counterpart or proteins or nucleic acids that have been chemically synthesized and are thus substantially uncontaminated by other proteins or nucleic acids. The term “isolated” may include proteins, nucleic acids, or cells that have been separated or purified from most other cellular components or organism components with which they are naturally accompanied (e.g., but not limited to, other cellular proteins, nucleic acids, or cellular or extracellular components).

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

The term “plant” includes whole plants, plant organs (e.g., leaves, stems, flowers, roots, reproductive organs, embryos, and parts thereof, etc.), seedlings, seeds and plant cells, and progeny thereof. The class of plants which can be used in the method of the invention is generally as broad as the class of higher plants amenable to transformation techniques. including angiosperms (monocotyledonous and dicotyledonous plants), as well as gymnosperms. It includes plants of a variety of ploidy levels, including polyploid (e.g., octoploid), diploid, haploid, and hemizygous.

The term “locus” refers to a position in the genome that corresponds to a measurable characteristic (e.g., a trait) or gene. A locus can be a genomic region or section of DNA (the locus) which correlates with a variation in a phenotype. A locus can comprise a single or multiple genes or other genetic information within a contiguous genomic region or linkage group.

A “marker” or “genetic marker” refers to a gene or nucleotide sequence that can be used to identify the presence or location of a trait determinant, locus, gene, and/or allele. A genetic marker may be described as a variation at a given genomic locus. A genetic marker may be a short DNA sequence, such as a sequence surrounding a single base-pair change (for example as in a single nucleotide polymorphism (SNP)), or a longer DNA sequence, for example, a microsatellite/simple sequence repeat (SSR)). A “marker allele” refers to the version of the marker that is present in a particular individual. A genetic marker can be used to identify individuals, genes, or loci in an off-spring originating from an individual parent. An “FaRCa1 marker” is a genetic marker that is linked, closely linked, tightly linked, or extremely tightly linked to the 163.0 gene or the 163.26 gene.

“Linked” refers to one or more genes or markers that are located within about 65 megabases (Mb) of one another on the same chromosome. Thus, two “linked” genes or markers may be separated, for example, by about 65 Mb, about 60 Mb, about 55 Mb, about 50 Mb, about 45 Mb, about 40 Mb, about 35 Mb, about 30 Mb, about 25 Mb, about 20 Mb, about 15 Mb, about 10 Mb, about 9.0. Mb, about 8.0 Mb, about 7.0 Mb, about 6.0 Mb, about 5.2 Mb, about 4.0 Mb, about 3.0 Mb. about 2.0 Mb, about 1.0 Mb, or fewer Mb.

“Closely linked” refers to one or more genes or markers that are located within about 2.0 Mb of one another on the same chromosome. Thus, two closely linked genes or markers may be separated, for example, by about 2.00 Mb, about 1.95 Mb, about 1.90 Mb, about 1.85 Mb, about 1.80 Mb, about 1.75 Mb, about 1.70 Mb, about 1.65 Mb, about 1.60 Mb, about 1.55 Mb, about 1.50 Mb, about 1.45 Mb, about 1.40 Mb, about 1.35 Mb. about 1.30 Mb. about 1.25 Mb, about 1.20 Mb, about 1.15 Mb, about 1.10 Mb, about 1.05 Mb, about 1.00 Mb, about 0.95 Mb, about 0.90 Mb, about 0.85 Mb, about 0.80 Mb, about 0.75 Mb, about 0.70 Mb, about 0.65 Mb, about 0.60 Mb, about 0.55 Mb, about 0.50 Mb, about 0.45 Mb, about 0.40 Mb, about 0.35 Mb, about 0.30 Mb, about 0.25 Mb, about 0.20 Mb, about 0.15 Mb, about 0.10 Mb. about 0.05 Mb, about 0.025 Mb, or about 0.01 Mb

“Tightly linked” refers to one or more genes or markers that are located within about 1.0 Mb of one another on the same chromosome. Thus, two tightly linked genes or markers may be separated, for example, by about 1.00 Mb, about 0.95 Mb, about 0.90 Mb, about 0.85 Mb, about 0.80 Mb, about 0.75 Mb, about 0.70 Mb, about 0.65 Mb. about 0.60 Mb. about 0.55 Mb about 0.5 Mb, about 0.45 Mb, about 0.4 Mb, about 0.35 Mb, about 0.3 Mb, about 0.25 Mb, about 0.2 Mb, about 0.15 Mb, about 0.1 Mb, or about 0.05 Mb.

“Extremely tightly linked” refers to one or more genes or markers that are located within about 100 kb of one another on the same chromosome. Thus, two extremely tightly linked genes or markers may be separated, for example, by about 100 kb, about 95 kb, about 90 kb, about 85 kb, about 80 kb, about 75 kb, about 70 kb, about 65 kb, about 60 kb, about 55 kb, about 50 kb, about 45 kb, about 40 kb, about 35 kb, about 30 kb, about 25 kb, about 20 kb, about 15 kb, about 10 kb, about 5 kb, or about 1 kb.

“Introgression” or “introgressing” of a locus means introduction of a locus from a donor plant comprising the locus of interest into a recipient plant by standard breeding techniques, wherein selection can be done phenotypically by means of observation of a plant characteristic including, but not limited to, characteristics such as the intemodal length or plant height, or selection can be done with the use of markers through marker-assisted breeding, or combinations of these. The process of introgressing is often referred to as “backcrossing” when the process is repeated two or more times. In introgressing or backcrossing, the “donor” parent refers to the parental plant with the desired gene or locus to be introgressed. The “recipient” parent (used one or more times) or “recurrent” parent (used two or more times) refers to the parental plant into which the gene or locus is being introgressed. Selection is started in the F1 or any further generation from a cross between the recipient plant and the donor plant, suitably by using markers as identified herein. The skilled person is, however, familiar with creating and using new molecular markers that can identify or are linked to a locus of interest.

A “homolog” or “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologs (orthologous sequences) and paralogs (paralogous sequences). Homologous genes, for example, typically descend from a common ancestral DNA sequence, either through a speciation event (orthologous genes) or a genetic duplication event (paralogous genes). “Orthologous” genes are genes in different species that evolved from a common ancestral gene by speciation. Orthologs typically retain the same function in the course of evolution. “Paralogous” genes include genes related by duplication within a genome. Paralogs can evolve new functions in the course of evolution.

A “heterologous” sequence is a sequence which is not normally present in a cell, genome, or gene in the genetic context in which the sequence is currently found. A heterologous sequence can be a sequence derived from the same gene and/or cell type, but introduced into the cell or a similar cell in a different context, such as on an expression vector or in a different chromosomal location or with a different promoter. A heterologous sequence can be a sequence derived from a different gene or species than a reference gene or species. A heterologous sequence can be from a homologous gene from a different species, from a different gene in the same species, or from a different gene from a different species. For example, a regulatory sequence may be heterologous in that it is linked to a different coding sequence relative to the native regulatory sequence.

Compositions or methods “comprising” or “including” one or more recited elements may include other elements not specifically recited. For example, a composition that “comprises” or “includes” a marker may contain the marker alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which it does not.

The term “and/or” refers to and encompasses any and all possible combinations of one or more of the associated listed items, as well as the lack of combinations when interpreted in the alternative (“or”). The term “or” refers to any one member of a particular list and also includes any combination of members of that list.

The singular forms of the articles “a.” “an.” and “the” include plural references unless the context clearly dictates otherwise. For example, the term “a marker” or “at least one marker” can include a plurality of markers, including mixtures thereof.

“Colletotrichum acutatum” or “C. acutatum” is a plant pathogen and endophyte. It is the organism that causes the most destructive fungal disease, anthracnose, of lupin species worldwide. C. acutatum has a broad host range, but is most important in strawberries.

“Anthracnose fruit rot” (AFR) is a disease of strawberry with all parts of the plant (fruit, crowns, leaves, petioles, and runners) being susceptible to the pathogen. Three related species of the fungus Colletotrichum, including C. aculatum, C. gloeosporioides, and C. fragariae can be associated with anthracnose. C. acutatum is the main pathogen associated with the AFR. Anthracnose fruit rot appears as brown to black, water-soaked spots on green and ripe fruit. Firm, sunken brown to black lesions can develop over time depending on the prevalent relative humidity at the time of disease development. Pink, salmon, or orange-colored masses of spores may form in the lesion under humid conditions where lesions may appear less sunken and brownish. Under dry conditions, lesions appear more sunken and black, and the entire fruit may dry up to be mummified.

“Anthracnose root necrosis” (ARN) is a disease of strawberry characterized by brown to black decayed roots and crown necrosis by reddish to brown necrotic crown tissue after cutting diseased crowns. ARC can cause stunting, wilting, and, in some cases. collapse of plants in the field. ARN is primarily caused by C. acutatum infection.

The term “FaRCa1 locus” refers to a large-effect quantitative trait locus for AFR and/or ARN resistance. The FaRCa1 locus comprises a region of 165 kb located on chromosome Fvb6-3 from the ‘Camarosa’ reference genome between 16,238,392 bp and 16,403,945 bp (FIG. 4A).

The term “trio binning” refers to a method of genome assembly that takes advantage of heterozygosity instead of trying to remove it. Most current technologies attempt to collapse parental haplotypes into a composite, haploid sequence, introducing erroneous duplications through mis-assembly of heterozygous sites as separate genomic regions. This problem is exacerbated in highly heterozygous genomes, particularly in cases such as allo-octoploid strawberry, resulting in fragmented and inflated assemblies that impede downstream analyses. In trio binning, a family trio is sequenced with short reads for both parents and long reads for an F1 offspring. Parent-specific k-mer markers are then identified from the parental reads and used to assign offspring reads into maternal and paternal bins before assembling each parental haploid genome separately. The ability of trio binning to accurately distinguish parental haplotypes increases at greater heterozygosity.

An “RNA-guided DNA endonuclease” is an enzyme (endonuclease) that uses RNA-DNA complementarity to identify target sites for sequence-specific double-stranded DNA (dsDNA) cleavage. An RNA-guided DNA endonuclease may be, but is not limited to, a zCas9 nuclease. a Cas9 nuclease. type II Cas nuclease, an nCas9 nuclease, a type V Cas nuclease, a Cas12a nuclease, a Casl2b nuclease, a Casl2c nuclease, a CasY nuclease, a CasX nuclease, a Cas12i nuclease, or an engineered RNA-guided DNA endonuclease.

A “guide RNA” (gRNA) comprises an RNA sequence (tracrRNA) bound by Cas and a spacer sequence (crRNA) that hybridizes to a target sequence and defines the genomic target to be modified. The tracrRNA and crRNA may be linked to form a “single chimeric guide RNA” (sgRNA).

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297: Barrangou et al. (2012) Annu Rev Food Sci Technol 3:143-162; Jinek et al. (2012) Science 337:816-821, Cong et al. (2013) Science 339:819-823; Mali et al. (2013) Science 339: 823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e.. target sequence). Non-limiting examples of PAMs include NGG, NNGRRT, NN[A/C/T]RRT, NGAN, NGCG, NGAG, NGNG, NGC, and NGA.

A “trans-activating CRISPR RNA” (tracrRNA) is an RNA species facilitates binding of the RNA-guided DNA endonuclease (e.g., Cas) to the guide RNA.

A “CRISPR system” comprises a guide RNA, either as a crRNA and a tracrRNA (dual guide RNA) or an sgRNA, and RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.

A “DNA donor template” is a nucleic acid, such as a single stranded DNA, linear double strand DNA, plasmid DNA or AAV sequence that provides the homology necessary for precise repair of a double-strand break. A DNA donor template contains two regions of homology, one region of homology to either side of the double strand break. A DNA donor template can also contain a heterologous sequence between the two regions of homology. Each region of homology can be about 30 to about 100 nucleotides in length.

A “regenerant” is a plant produced from a plant tissue cell, such as a genetically modified plant tissue cell.

### IL. Overview

Identification, sequencing, and functional characterization of genes that control resistance to C. acutatum are of great interest in strawberry breeding. Complete sequence of resistance gene(s) also allows the characterization of the protein transcribed, insights into resistance mechanisms, the design of functional markers, and implementation of new breeding techniques such as gene editing, cisgenesis, and epigenetic approaches. To accomplish all these endeavors, advanced genomic resources and molecular tools for strawberry are required. The complete reference genome and annotation for the octoploid cultivar ‘Camarosa’. also referred to as F.×ananassa genome v1.0. constitutes a very useful genomic advancement in strawberry. This assembly has 805,488,706 bp distributed across 28 chromosomes and represents 99% of the estimated genome size. In total, 108,087 protein-coding genes and 30,703 genes encoding long noncoding RNAs were annotated. With the goal of updating the F.×ananassa genome by utilizing one PacBio full-length RNA library and ninety-two Illumina RNA-Seq libraries, a structural and functional reannotation was completed. The updated version, F×ananassa genome v1.a2, has a total of 108,447 gene models from which 19,174 genes were modified, 360 are new, and 11,044 showed alternatively spliced isoforms.

Described is a high-quality haplotype-phased genome assembly of a major Florida commercial cultivar, ‘Florida Brilliance’ (FaFBI) without parental information. The available haplotype-resolved octoploid strawberry (Fragaria×ananassa Duch.) (2n=8×=56) genomes were all built with the trio-binning pipeline. supplied with parental short-reads. Using Pacific Biosciences (PacBio) long reads and high-throughput chromatic capture (Hi-C) data, the telomere-to-telomere phased genome assembly was completed. The N50 continuity of the phased-1 and phased-2 assemblies were 23.7 Mb and 26.6 Mb before scaffolding and gap-filling, respectively. All 56 pseudo-chromosomes from phased-1 and phased-2 assembly contained putative telomere sequences at the 5′ and/or 3′ ends. The high level of collinearity between the final haploid assemblies of the genome was confirmed by high-density genetic linkage map containing 10,269 SNP markers. Genome completeness was further confirmed by Meryl-based consensus quality (QV). LTR assembly Index (LAI) score for entire genome assembly was 19.72, indicating a gold-standard quality. Moreover, the BUSCO analysis detected over 99% of conserved genes in the combined phased-1 and pahsed-2 assemblies. Each haploid assembly of ‘Florida Brilliance’ was annotated using Iso-Seq data from six different strawberry tissues and RNA-Seq data representing various F.×ananassa tissues from the NCBI sequence read archive (SRA).

Bacterial artificial clone (BAC) libraries for strawberry genotypes 11.77-96, ‘Florida Brilliance’ and 14.101-225 constructed by Amplicon Express using restriction enzymes HindIII and BamHI are available in the UF strawberry molecular genetics and genomics laboratory. Fragments obtained from these six libraries were cloned into vector pCC1 BAC (EPICENTRE, Madison, WI. USA) and transformed in the host E. coli cells Phage Restraint DH10B per genotype. In addition, a contig-level assembly using 20× PacBio data for cultivar ‘Florida Brilliance’ that was preliminarily assembled using Canu assembler (correcter error rate=0.105, cor out coverage=200) and assembled contigs for cultivar ‘Florida Brilliance’ using HiFi data are currently available. Additionally, using resistance gene enrichment sequencing (RenSeq), the distribution of disease resistance genes (R-gene) in the genomes of the octoploid strawberry. ‘Camarosa’ and two diploid ancestral relatives (F. vesca and F. iinumae) were extensively characterized. From the octoploid fruit eQTL analysis, about 76 putative R-genes were identified.

Transient gene expression is an effective genomics tool for validating functions of candidate genes via overexpression or RNAi-mediated gene silencing in plants. The optimization for transient transformation for gene functional analysis in strawberry fruit has previously been accomplished using eGFP and GUS as reporter genes. The optimal conditions for successful transient gene manipulation withAgrobacteriumn tumefaciens required detached strawberry fruits in large green to white stage. Maximum level of gene expression was observed four to six days after infiltration of A. tumefaciens harboring a gene of interest, and fruits were incubated at 20-25° C. Additionally, best results were obtained when the fruit was fully infiltrated with A. tumefaciens146.

The cultivar ‘Camarosa’ is susceptible to C. acutatum and is homozygous (AA) for the 9-bp InDel-based HRM markers at the FaRCa1 locus, which presents challenges for finding the gene or genes responsible for resistance to C. acutatum, because it is only the reference genome for octoploid strawberry. Nevertheless, the available ‘Camarosa’ reference genome and annotations serves as an extremely valuable guide for defining the FaCal region. Advanced selections 11.77-96 and 14.101-225, and cultivar ‘Florida Brilliance’ are heterozygous (AB) for the 9-bp InDel-based HRM marker at the FaRCa1 locus where A and B corresponds to the susceptible and resistant allele, respectively. Therefore, resistant and susceptible alleles for the FaRCa1 locus can be potentially differentiated with BAC library screening and available for the assemblies of ‘Florida Brilliance’.

A major resistance locus, named FaRCa1, with a peak located between 16,238,392 bp and 16,403,945 bp on chromosome Fvb6-3 from the ‘Camarosa’ reference genome, explains at least 50% of the phenotypic variation for anthracnose fruit rot (AFR) resistance across trials and seasons. FaRCa1 also confers moderate levels of resistance to anthracnose root necrosis (ARN). The UF strawberry breeding program has incorporated a 9-bp InDel-based marker in FaRCa1 into the high throughput seedling selection process for C. acutatum resistance. However, the 9-bp InDel-based marker is located at an unknown distance from the causal gene controlling resistance to C. acutatum. Presence of outliers in boxplots showing AFR and ARN incidence by marker genotype are likely the result of recombination events between the InDel-based marker and the still-unknown causal gene.

Using the above-described gold-standard reference genome of ‘Florida Brilliance’, receptor kinase genes regulating resistance to anthracnose fruit rot in octoploid cultivated strawberry were identified.

Described are compositions for genetically modifying a strawberry plant to increase resistance to AFR and/or ARN. Also described are methods of using the compositions for producing plants having an AFR-resistant and/or ANR-resistant phenotype. In some embodiments, the plant is a Fragaria plant. A Fragaria plant can be any member of the Fragaria genus suitable for commercial strawberry production. A Fragaria plant can be a diploid species, a tetraploid species, a hexaploid species, an octoploid species, or any other ploidy species or hybrid thereof. AFragaria plant can be, but is not limited to, Fragaria vesca. Fragaria×bifera, Fragaria×bringhurstii, Fragaria virginiana, Fragaria chiloensis, or Fragaria×ananassa. In some embodiments, the Fragaria plant is a Fragaria×ananassa plant.

An “AFR resistance-associated locus” or “C. acutatum resistance-associated locus” comprises a locus that corresponds to the trait (phenotype) of measurably reduced AFR disease severity incidence in a plant following infestation with C. acutatum. One such non-limiting example of a AFR resistance-associated locus is the FaRCa1 locus. Specific genes within the FaRCa1 locus that correlate with AFR and/or ARN resistance include the 163.0 gene and the 163.26 gene.

The FaRCa1 locus can include coding and non-coding sequence. Non-coding sequence includes, but is not limited to, regulator sequences, intron sequences and scaffold sequences.

A “163.0 gene” comprises a polynucleotide encoding a putative rust resistance kinase, as described in detail elsewhere in the application. In some embodiments, the 163.0 gene comprises SEQ ID NO: 1. In some embodiments, the 163.0 gene or coding sequence encodes SEQ ID NO: 2. In some embodiments, the 163.0 protein comprises SEQ ID NO: 2. In some embodiments, a 163.0 nucleic acid comprises a sequence that encodes the amino acid sequence of SEQ ID NO: 2.

A “163.26 gene” comprises a polynucleotide encoding a putative rust resistance kinase, as described in detail elsewhere in the application. In some embodiments, the 163.26 gene comprises SEQ ID NO: 3. In some embodiments, the 163.26 gene or coding sequence encodes SEQ ID NO: 4. In some embodiments, the 163.26 protein comprises SEQ ID NO: 4. In some embodiments, a 163.26 nucleic acid comprises a sequence that encodes the amino acid sequence of SEQ ID NO: 4.

The 163.0 and 163.26 genes may be collectively referred to as “AFR resistance genes” or “C. acutatum resistance-associated genes.” The FaRCa1 locus may be referred to as “AFR resistance-associated locus” or “C. acutatum resistance-associated locus.” The 163.0 and 163.26 genes may also be referred to as “ARN resistance genes.” Described are methods of increasing resistance of strawberry plants to AFR and/or ARN comprising expressing in the strawberry plants one or more heterologous genes selected from the group consisting of: 163.0 and 163.26. The methods can be used to improve strawberry crop yield in areas where C. acutatum is present. In some embodiments, loss of crop yield in genetically modified resistant plants may be reduced by at least 5%, at least 10%, at least 15%, at least 20%, at least 25%, at least 30%, at least 40%, at least 50%, at least 60%, at least 70%, at least 80%, at least 90%, or at least 95%, or up to 100% compared to C. acutatum susceptible Fragaria cultivars. The methods can be used to decrease the incidence or severity of AFR and/or ARN by at least 10%, at least 5%, at least 10%, at least 15%, at least 20%, at least 25%, at least 30%, at least 40%, at least 50%, at least 60%, at least 70%, at least 80%, at least 90%, or at least 95%, or up to 100% compared to C. acutatum susceptible Fragaria cultivars.

In some embodiments, constructs and systems for unaided homologous recombination. recombinase-based insertion, and DNA repair-based insertion targeted genetic modification of Fragaria plants are described. The constructs and systems can be used to insert a heterologous 163.0 or 163.26 gene into a strawberry plant. The constructs and systems can also be used to replace an endogenous allele of 163.0 or 163.26 that does not confer AFR and/or ARN resistance with all or a portion of SEQ ID NO: 1 and SEQ ID NO: 3, respectively.

In some embodiments, nucleic acids for producing AFR and/or ARN resistant plants using CRISPR systems are described. The CRISPR systems can target one or more of the AFR and/or ARN resistance genes in the FaRCa1 locus. The nucleic acids include, but are not limited to, nucleic acids comprising crRNAs or gRNAs and nucleic acids encoding crRNAs or gRNAs.

In some embodiments, nucleic acids for producing AFR and/or ARN resistant plants using CRISPR systems are described. The CRISPR systems can be used to knock in one or more of a heterologous 163.0 gene and a heterologous 163.26 gene into a Fragaria plant cell. The nucleic acids include, but are not limited to, nucleic acids comprising crRNAs or gRNAs, nucleic acids encoding crRNAs or gRNAs, and DNA donor template nucleic acids. The CRISPR systems can knock in one or more of a heterologous 163.0 gene and a heterologous 163.26 gene unto the FaRCa1 locus of the plant genome or into an alternative site in the plant genome.

In some embodiments, nucleic acids for producing AFR and/or ARN resistant plants using CRISPR systems are described. The CRISPR systems can be used to modify an endogenous 163.0 gene and/or an endogenous 163.26 gene into a Fragaria plant or plant cell. The nucleic acids include, but are not limited to, nucleic acids comprising crRNAs or gRNAs, nucleic acids encoding crRNAs or gRNAs, and DNA donor template nucleic acids. In some embodiments, the endogenous 163.0 gene does not encode SEQ ID NO: 2 and the modified 163.0 gene encodes SEQ ID NO: 2. In some embodiments, the endogenous 163.26 gene does not encode SEQ ID NO: 4 and the modified 163.26 gene encodes SEQ ID NO: 4.

In some embodiments, methods of producing AFR and/or ARN resistant Fragaria plants and methods of genetically modifying a Fragaria plant to produce a AFR and/or ARN resistant plant using a CRISPR system are described.

In some embodiments. Fragaria plants having a AFR and/or ARN resistant phenotype produced using any one or more of the described CRISPR constructs are described.

The described FaRCa1 locus can be targeted to genetically modify Fragaria plants to yield a AFR and/or ARN resistant phenotype. Genetically modified Fragaria plants expressing or overexpressing one or more of 163.0 and 163.26 genes exhibit reduced severity of AFR and/or ARN disease incidence compared to the otherwise genetically identical plants.

### III. Targeted Genetic Modification

A targeted genetic modification can comprise a targeted alteration to a polynucleotide of interest including, for example, a targeted alteration to a target genomic locus on chromosome 6 of a Fragaria plant, a targeted alteration to the FaRCa1 locus in a Fragaria plant, or a targeted alteration a genomic location suitable for expression of a heterologous gene in a Fragaria plant. Such targeted modifications include, but are not limited to, additions of one or more nucleotides, deletions of one or more nucleotides, substitutions of one or more nucleotides, a knockout of the polynucleotide of interest or a portion thereof, a knock-in of a polynucleotide of interest or a portion thereof (e.g., a heterologous 163.0 gene, coding sequence, or fragment thereof and/or a heterologous 163.26 gene, coding sequence, or fragment thereof), a replacement of an endogenous nucleic acid sequence with a heterologous nucleic acid sequence, insertion of a regulatory sequence upstream or downstream of an endogenous gene (e.g., a 163.0 gene and/or a 163.26 gene) or a combination thereof. In certain embodiments, at least 1, 2, 3, 4. 5, 7, 8, 9, 10 or more nucleotides are changed to form the targeted genomic modification. In some embodiments, the targeted genetic modification comprises a targeted DNA insertion of one or more polynucleotides encoding one or more AFR and/or ARN resistance genes.

Various methods can be used to generate the targeted modification in the polynucleotide or Fragaria plant genome of interest. Methods of obtaining the targeted genetic modifications as described in the instant application can comprise unaided homologous recombination, recombinase-based insertion, and DNA repair-based insertion and are known in the art (Dong et al., 2021. PNAS. 118(22) e2004834117). Recombinase-based insertion can comprise systems and constructs involving site-specific recombinases, including but not limited to, Cre:loxP systems, Flp:FRT systems, Dre:rox systems, VCre:loxVsystems, Gin:gix systems, Bxbl:attP attB systems, phiC3l:attP attB systems. DNA repair-based insertion methods rely upon the activity of a nuclease agent.

The term “recognition site for a nuclease agent” includes a DNA sequence at which a nick or double-strand break is induced by a nuclease agent. The recognition site for a nuclease agent can be endogenous (or native) to the cell or the recognition site can be exogenous to the cell. In specific embodiments, the recognition site is exogenous to the cell and thereby is not naturally occurring in the genome of the cell. In still further embodiments, the recognition site is exogenous to the cell and to the polynucleotides of interest that one desires to be positioned at the target locus. In further embodiments, the exogenous or endogenous recognition site is present only once in the genome of the host cell. In specific embodiments, an endogenous or native site that occurs only once within the genome is identified. Such a site can then be used to design nuclease agents that will produce a nick or double-strand break at the endogenous recognition site.

The length of the recognition site can vary, and includes, for example, recognition sites that are about 30-36 bp for a zinc finger nuclease (ZFN) pair (i.e., about 15-18 bp for each ZFN), about 36 bp for a Transcription Activator-Like Effector Nuclease (TALEN), or about 20 bp for a CRISPR/Cas9 guide RNA.

In one embodiment, each monomer of the nuclease agent recognizes a recognition site of at least 9 nucleotides. In other embodiments, the recognition site is from about 9 to about 12 nucleotides in length, from about 12 to about 15 nucleotides in length, from about 15 to about 18 nucleotides in length, or from about 18 to about 21 nucleotides in length, and any combination of such subranges (e.g., 9-18 nucleotides). It is recognized that a given nuclease agent can bind the recognition site and cleave that binding site or alternatively, the nuclease agent can bind to a sequence that is different from the recognition site. Moreover, the term recognition site comprises both the nuclease agent binding site and the nick/cleavage site irrespective whether the nick/cleavage site is within or outside the nuclease agent binding site. In another variation, the cleavage by the nuclease agent can occur at nucleotide positions immediately opposite each other to produce a blunt end cut or, in other cases, the incisions can be staggered to produce single-stranded overhangs, also called “sticky ends”, which can be either 5′ overhangs, or 3′ overhangs.

Any nuclease agent that induces a nick or double-strand break into a desired recognition site can be used in the methods and compositions disclosed herein. A naturally occurring or native nuclease agent can be employed so long as the nuclease agent induces a nick or double-strand break in a desired recognition site. Alternatively, a modified or engineered nuclease agent can be employed. An “engineered nuclease agent” includes a nuclease that is engineered (modified or derived) from its native form to specifically recognize and induce a nick or double-strand break in the desired recognition site. Thus, an engineered nuclease agent can be derived from a native, naturally occurring nuclease agent or it can be artificially created or synthesized. The modification of the nuclease agent can be as little as one amino acid in a protein cleavage agent or one nucleotide in a nucleic acid cleavage agent. In some embodiments, the engineered nuclease induces a nick or double-strand break in a recognition site, wherein the recognition site was not a sequence that would have been recognized by a native (non-engineered or non-modified) nuclease agent. Producing a nick or double-strand break in a recognition site or other DNA can be referred to herein as “cutting” or “cleaving” the recognition site or other DNA. Assays to measure the double-strand break of a recognition site by a nuclease agent are known in the art (e.g., TaqMann qPCR assay, Frendewey D. et al., Methods in Enzymology, 2010, 476:295-307, which is incorporated by reference herein in its entirety).

In some embodiments, the recognition site is positioned within the polynucleotide encoding a selection marker. Such a position can be located within the coding region of the selection marker or within the regulatory regions, which influence the expression of the selection marker. Thus, a recognition site of the nuclease agent can be located in an intron of the selection marker, a promoter, an enhancer, a regulatory region, or any non-protein-coding region of the polynucleotide encoding the selection marker. In specific embodiments, a nick or double-strand break at the recognition site disrupts the activity of the selection marker. Methods to assay for the presence or absence of a functional selection marker are known.

In one embodiment, the nuclease agent is a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107: Scholze & Boch (2010) Virulence 1:428-432; Christian et al. Genetics (2010) 186:757-761; Li et al. (2010) Nuc. Acids Res. (2010) doi:10.1093/nar/gkg704; and Miller et al. (2011) Nature Biotechnology 29:143-148; all of which are herein incorporated by reference.

Examples of suitable TAL nucleases, and methods for preparing suitable TAL nucleases, are disclosed, e.g., in US Patent Application No. 2011/0239315 A1, 2011/0269234 A1, 2011/0145940 A1, 2003/0232410 A1, 2005/0208489 A1, 2005/0026157 A1, 2005/0064474 A1. 2006/0188987 A1, and 2006/0063231 A1 (each hereby incorporated by reference). In various embodiments. TAL effector nucleases are engineered that cut in or near a target nucleic acid sequence in, e.g., a locus of interest or a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors as described herein.

In one embodiment, each monomer of the TALEN comprises 33-35 TAL repeats that recognize a single base pair via two hypervariable residues. In one embodiment, the nuclease agent is a chimeric protein comprising a TAL repeat-based DNA binding domain operably linked to an independent nuclease. In one embodiment, the independent nuclease is a FokI endonuclease. In one embodiment, the nuclease agent comprises a first TAL-repeat-based DNA binding domain and a second TAL-repeat-based DNA binding domain, wherein each of the first and the second TAL-repeat-based DNA binding domain is operably linked to a FokI nuclease. wherein the first and the second TAL-repeat-based DNA binding domain recognize two contiguous target DNA sequences in each strand of the target DNA sequence separated by a spacer sequence of varying length (12-20 bp), and wherein the FokI nuclease subunits dimerize to create an active nuclease that makes a double strand break at a target sequence.

The nuclease agent employed in the various methods and compositions disclosed herein can further comprise a zinc-finger nuclease (ZFN). In one embodiment, each monomer of the ZFN comprises 3 or more zinc finger-based DNA binding domains, wherein each zinc finger-based DNA binding domain binds to a 3 bp subsite. In other embodiments, the ZFN is a chimeric protein comprising a zinc finger-based DNA binding domain operably linked to an independent nuclease. In one embodiment, the independent endonuclease is a FokI endonuclease. In one embodiment, the nuclease agent comprises a first ZFN and a second ZFN, wherein each of the first ZFN and the second ZFN is operably linked to a FokI nuclease subunit, wherein the first and the second ZFN recognize two contiguous target DNA sequences in each strand of the target DNA sequence separated by about 5-7 bp spacer, and wherein the FokI nuclease subunits dimerize to create an active nuclease to make a double strand break. See, for example, US20060246567; US20080182332; US20020081614; US20030021776; WO/2002/057308A2; US20130123484; US20100291048; WO/2011/017293A2; and Gaj et al. (2013) Trends in Biotechnology, 31(7):397-405 each of which is herein incorporated by reference.

In still another embodiment, the nuclease agent is a meganuclease. Meganucleases have been classified into four families based on conserved sequence motifs, the families are the LAGLIDADG. GIY-YIG. H-N-H, and His-Cys box families. These motifs participate in the coordination of metal ions and hydrolysis of phosphodiester bonds. Meganucleases are notable for their long recognition sites, and for tolerating some sequence polymorphisms in their DNA substrates. Meganuclease domains, structure and function are known, see for example. Guhan and Muniyappa (2003) Crit Rev Biochem Mol Biol 38:199-248: Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764. In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346.

Any meganuclease can be used herein, including, but not limited to, I-SceI, I-SceII, I-SceIII, I-SceIV, I-SceV, I-SceVI, I-SceVII, I-CeuI, I-CeuAIIP, I-CreI, I-CrepsbIP, I-CrepsbIIP, I-CrepsbIIIP, I-CrepsbIVP. I-TliI, I-PpoI, PI-PspI, F-SceI, F-SceII, F-SuvJ, F-TevI, F-TevII, I-AmaI. I-Anil. I-ChuI. I-Cmoel. I-CpaI, I-CpaIl, I-CsmI. I-Cvul, I-CvuAIP, I-DdiI, I-DdilI, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorI, I-PorlIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I. I-SthPhiJP, I-SthPhiST3P. I-SthPhiSTe3bP. I-TdeIP, I-TevI, I-TevII, I-TevIII, I-UarAP, I-UarHGPAIP. I-UarHGPA13P, I-VinIP, I-ZbiIP, PI-MtuI, PI-MtuHIP PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

Nuclease agents can further comprise restriction endonucleases, which include Type I, Type II, Type III, and Type IV endonucleases. Type I and Type III restriction endonucleases recognize specific recognition sites, but typically cleave at a variable position from the nuclease binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington. DC).

The nuclease agent employed in the various methods and compositions can also comprise a CRISPR/Cas system. Described are nucleic acids for producing AFR and/or ARN resistant plants using a CRISPR (e.g., CRISPR/Cas) system. The described nucleic acids can be used to target modification of the FaRCa1 locus and/or to insert or express one or more AFR and/or ARN resistance genes in a plant.

A CRISPR system comprises an RNA-guided DNA endonuclease enzyme and a CRISPR RNA. In some embodiments, a CRISPR RNA is part of a guide RNA. In some embodiments, the RNA-guided DNA endonuclease enzyme is a Cas9 protein. In some embodiments, a CRISPR system comprises one or more nucleic acids encoding an RNA-guided DNA endonuclease enzyme (such as, but not limited to a Cas9 protein) and a guide RNA. A guide RNA can comprise a CRISPR RNA (crRNA) and a trans-activating CRISPR RNA (tracrRNA), either as separate molecules or a single chimeric guide RNA (sgRNA). The guide RNA contains a guide sequence having complementarity to a sequence in the target gene genomic region. The Cas protein can be introduced into the plant in the form of a protein or a nucleic acid (DNA or RNA) encoding the Cas protein (e.g., operably linked to a promoter expressible in the plant). The guide RNA can be introduced into the plant in the form of RNA or a DNA encoding the guide RNA (e.g.. operably linked to a promoter expressible in the plant). In some embodiments, the CRISPR system further includes a DNA donor template. In some embodiments, the CRISPR system can be delivered to a plant or plant cell via a bacterium. The bacterium can be, but is not limited to. Agrobacterium tumefaciens.

In some embodiments, the CRISPR system is designed knock in a 163.0 gene, coding sequence. or fragment thereof and/or a heterologous 163.26 gene. coding sequence. or a fragment thereof into a locus in the Fragaria plant suitable for expression of a heterologous gene (transgene). The locus can be the FaRCa1 locus or another locus. In some embodiments, the CRISPR system is designed insert one or more of a heterologous 163.0 gene, coding sequence. or fragment thereof and/or a heterologous 163.26 gene, coding sequence, or fragment thereof into the genome or a Fragaria plant thereby resulting in increased expression of the heterologous 163.0 gene, coding sequence, or fragment thereof and/or the heterologous 163.26 gene, coding sequence, or fragment thereof in the plant relative to a plant that does not contain the heterologous 163.0 gene. coding sequence, or fragment thereof and/or 163.26 gene. coding sequence, or fragment thereof.

In some embodiments, the CRISPR system is designed to target one or more of the described AFR and/or ARN resistance genes and/or regions of the FaRCa1 locus. In some embodiments, the CRISPR system is designed insert one or more a heterologous 163.0 gene, coding sequence, or fragment thereof and/or a heterologous 163.26 gene, coding sequence, or fragment thereof into the genome or a Fragaria plant thereby resulting in increased expression of the heterologous 163.0 gene, coding sequence, or fragment thereof and/or the heterologous 163.26 gene, coding sequence. or fragment thereof in the plant relative to a plant that does not contain the heterologous 163.0 gene, coding sequence, or fragment thereof and/or the heterologous 163.26 gene, coding sequence, or fragment thereof. In some embodiments, the CRISPR system is designed to modify the FaRCa1 locus to increase expression or activity of one or more of the endogenous 163.0 and/or 163.26 genes.

The CRISPR/Cas system can be, but is not limited to, a CRISPR class 1 system, CRISPR class 2 system, CRISPR/Cas system, a CRISPR/Cas9 system, a CRISPR/zCas9 system or CRISPR/Cas3 system.

Guide sequences suitable for forming gRNAs or crRNAs for CRISPR system mediated genetic modification of a FaRCa1 locus are described. Suitable guide sequences include 17-20 nucleotide sequences along the FaRCa1 locus, that are unique compared to the rest of the genome and immediately adjacent (5′) to a protospacer-adjacent motif (PAM) site. For the RNA-guided DNA endonuclease enzyme zCas9, a PAM site is NGG. Thus, any unique 17-20 nucleotide sequence immediately 5′ of a 5′-NGG-3′ the FarRCa1 locus can be used in forming a gRNA. In some embodiments, the guide sequence is 100% complementary to the target sequence. In some embodiments, the guide sequence is at least 90% or at least 95% complementary to the target sequence. In some embodiments, the guide sequence contains 0, 1, or 2 mismatches when hybridized to the target sequence. In some embodiments, a mismatch, if present, is located distal to the PAM, in the 5′ end of the guide sequence.

Additional guide sequence suitable for forming gRNAs or crRNAs for CRISPR system mediated genetic modification of Fragaria plants include 17-20 nucleotide sequences in the Fragaria genome that are unique compared to the rest of the genome and immediately adjacent (5′) to a protospacer-adjacent motif (PAM) site and suitable for insertion of a heterologous gene. Such sites are term safe harbor sites.

The DNA donor template contains sequence to be inserted into the genome of the plant. In some embodiments, the DNA donor template to be used with a CRISPR system comprises coding sequences for a heterologous 163.0 gene and/or a heterologous 163.26 gene (e.g., SEQ ID NO: 1 and/or 3, respectively, or a nucleic acid sequence that encodes SEQ ID NO: 2 and/or 4, respectively). The donor template can further comprise one or more regulator sequences operatively linked to the coding sequence that drives expression of the coding sequence in the plant cell. In some embodiments, the DNA donor template to be used with a CRISPR system comprises sequence for modifying the endogenous 163.0 and/or 163.26 genes to increase expression of activity of the endogenous 163.0 and/or 163.26 genes.

DNA donor templates further comprise 5′ and 3′ homology regions located 5′ and 3′ to the sequence to be inserted into the genome. The homology regions comprise about 30 to about 100 nucleotides that are complementary to a corresponding number of nucleotides in the genome on either side of the double strand break created by the CRISPR nuclease.

The DNA donor template can be provided a single strand DNA, double strand DNA, plasmid DNA, or viral vector DNA (e.g., adeno-associated vector DNA).

It is understood that RNA equivalents of any listed DNA sequences, substituting uracils (U) for thymines (T). may be used. An “RNA equivalent” is an RNA molecule having essentially the same complementary base pair hybridization properties as the listed DNA sequence.

CRISPR modification of a AFR and/or ARN resistance-associated locus or other genomic site is not limited to the CRISPR/zCas9 system. Other CRISPR systems using different nucleases and having different PAM sequence requirements are known in the art. PAM sequences vary by the species of RNA-guided DNA endonuclease. For example, Class 2 CRISPR-Cas type II endonuclease derived from S. pyogenes utilizes an NGG PAM sequence located on the immediate 3′ end of the guide sequence. Other PAM sequences include, but are not limited to, NNNNGATT (Neisseria meningitidis). NNAGAA (Streptococcus thermophilus), and NAAAAC (Treponema denticola). Guide sequences for CRISPR systems having nucleases with different PAM sequence requirements are identified as described above for zCas9, substituting the different PAM sequences.

Two or more guide RNAs can used with the same RNA-guided DNA endonuclease (e.g.. Cas nuclease) or different RNA-guided DNA endonucleases.

In some embodiments, two or more gRNAs targeting the insertion of two or more different AFR and/or ARN resistance genes are used. The two or more gRNAs can be used with the same RNA-guided DNA endonuclease or different RNA-guided DNA endonucleases.

Guide RNAs for modification of AFR and/or ARN resistance-associated loci in other Fragaria plants and other plants susceptible to C. acuatuin infestation, including but not limited to such economically important plants as apple, pear, stone fruits (e.g., peaches, plums, etc.), and ornamentals are generated in a similar manner by identifying the corresponding ortholog sequences of the AFR and/or ARN resistance-associated loci and/or genes and selecting target sequences as described above.

Any of the above-described guide RNAs can be provided as an RNA or a DNA encoding the RNA.

In some embodiments, a CRISPR system comprises one or more guide RNAs and a nucleic acid encoding an RNA-guided DNA endonuclease. In some embodiments, a CRISPR system comprises one or more guide RNAs, a nucleic acid encoding an RNA-guided DNA endonuclease, and a DNA donor template.

In some embodiments, a CRISPR system comprises one or more guide RNAs and a one or more nucleic acids encoding two or more different RNA-guided DNA endonucleases. In some embodiments, a CRISPR system comprises one or more guide RNAs, one or more nucleic acids encoding two or more different RNA-guided DNA endonucleases, and at least one DNA donor template.

In some embodiments, a CRISPR system comprises a guide RNA and an RNA-guided DNA endonuclease in a complex. In some embodiments, a CRISPR system comprises a guide two or more RNAs each in a complex with an RNA-guided DNA endonuclease. In some embodiments, a CRISPR system comprises a guide RNA and an RNA-guided DNA endonuclease in a complex and a DNA donor template.

### IV. Modified Plants

Methods of producing AFR and/or ARN resistant plants and methods of genetically modifying a plant to produce a AFR and/or ARN resistant plant using a CRISPR system are described. Other methods known in the art may also be used to produce the modified plants expressing the 163.0 or 163.26 genes.

Described are methods of generating genetically modified AFR and/or ARN resistant plants comprising introducing into a plant, a plant tissue, or a plant cell, one or more of the described CRISPR systems. In some embodiments, genetically modified AFR and/or ARN resistant plants created using a CRISPR system are described. In some embodiments, the CRISPR system is a CRISPR/Cas system.

In some embodiments, methods are described for producing a AFR and/or ARN resistant strawberry plant, the methods comprising the step of introducing into the plant one or more of the described CRISPR systems.

Nucleic acids may be introduced into a plant cell or cells using a number of methods known in the art, including but not limited to electroporation, DNA bombardment or biolistic approaches, microinjection, via the use of various DNA-based vectors such as Agrobacterium tumefaciens and Agrobacterium rhizogenes vectors, and CRISPR or CRISPR/Cas9. Once a plant cell has been successfully transformed, it may be cultivated to regenerate a transgenic plant (regenerant).

Various methods for introducing the transgene expression vector constructs of the invention into a plant or plant cell are well known to those skilled in the art, and any method capable of transforming the target plant or plant cell may be utilized.

In some embodiments, Agrobacterium tumefaciens is used to deliver CRISPR system nucleic acids to a plant. Agrobacterium-mediated transformation of a large number of plants are extensively described in the literature (see, for example, Agrobacterium Protocols, Wan, ed., Humana Press, 2^{nd} edition, 2006). Various methods for introducing DNA into Agrobacteria are known, including electroporation, freeze/thaw methods, and triparental mating. In some embodiments, a pMON316-based vector is used in the leaf disc transformation system of Horsch et al. Other commonly used transformation methods include, but are not limited to, microprojectile bombardment, biolistic transformation, and protoplast transformation of naked DNA by calcium, polyethylene glycol (PEG) or electroporation (Paszkowski et al., 1984. EMBO J. 3: 2727-2722; Potrykus et al., 1985, Mol. Gen. Genet. 199: 169-177: Fromm et al., 1985, Proc. Nat. Acad. Sci. USA 82: 5824-5828: Shimamoto et al., 1989, Nature, 338: 274-276.

To transgenic plants may be used to generate subsequent generations (e.g., Ti, T2, etc.) by selfing of primary or secondary transformants, or by sexual crossing of primary or secondary transformants with other plants (transformed or untransformed).

The described CRISPR systems can be used to genetically modify or introduce one or more AFR and/or ARN resistance genes in a plant. The plant can be a plant having another trait of interest. Delivery of the CRISPR system leads to insertions near the target sequence, resulting in expression of AFR and/or ARN resistance genes in the plant. Introducing a AFR and/or ARN resistant phenotype into a plant having a desired trait, using the described CRISPR systems and methods, may result in a cost savings for plant developers because such methods eliminate traditional plant breeding.

In some embodiments, the described CRISPR systems can be used to genetically modify or introduce 1. 2. 3, or more AFR and/or ARN resistance genes in a plant.

In some embodiments, the CIRSPR system is used to modify or introduce one or more AFR and/or ARN resistance-associated loci into a transgenic strawberry line. The transgenic strawberry line can also contain one or more genes for herbicide tolerance, increased yield, insect control, other fungal disease resistance, virus resistance, bacterial disease resistance, germination and/or seedling growth control, enhanced animal and/or human nutrition, improved processing traits, or improved flavor, among others.

Plants produced using the described CRISPR systems (having one of more of 163.0 and 163.26 and/or other AFR and/or ARN resistance genes and/or AFR and/or ARN resistance-associated loci) have a AFR and/or ARN resistant phenotype. The AFR and/or ARN resistant plants can produce similar sizes and quantities of fruit to an otherwise genetically similar plants lacking the genes and/or loci that confer resistance to C. acutatum and AFR and/or ARN. In some embodiments, the AFR and/or ARN resistant plants produce fruits at a yield of greater than 50%, greater than 60%, greater than 70%, greater than 80%, greater than 90% of the yield of an otherwise genetically similar plant lacking the genes and/or loci that confer resistance to C. acutatum and AFR and/or ARN when grown under the same conditions. In some embodiments, the AFR and/or ARN resistant plants produce fruits at a higher yield than an otherwise genetically similar plant lacking the genes and/or loci that confer resistance to C. acutatum and AFR and/or ARN when grown under the same conditions. In some embodiments, the AFR and/or ARN resistant plants produce fruits having an average size that is greater than 50%, greater than 60%, greater than 70%, greater than 80%, or greater than 90% of the average size of fruits produced by an otherwise genetically similar plant lacking the genes and/or loci that confer resistance to C. acutatum and AFR and/or ARN when grown under the same conditions. In some embodiments, the AFR and/or ARN resistant plants produce fruits having an average size that is greater than an otherwise genetically similar plant lacking the genes and/or loci that confer resistance to C. acutatum and AFR and/or ARN when grown under the same conditions. In some embodiments, the AFR and/or ARN resistant plants produce fruits having an average weight that is greater than 50%, greater than 60%, greater than 70%, greater than 80%, or greater than 90% of the average weight of fruits produced by an otherwise genetically similar plant lacking the genes and/or loci that confer resistance to C. acutatum and AFR and/or ARN when grown under the same conditions. In some embodiments, the AFR and/or ARN resistant plants produce fruits having an average weight that is greater than an otherwise genetically similar plant lacking the genes and/or loci that confer resistance to C. acutatum and AFR and/or ARN when grown under the same conditions. In some embodiments, the AFR and/or ARN resistant plants have decrease incidence or severity of AFR and/or ARN compared to an otherwise genetically similar plant lacking the genes and/or loci that confer resistance to C. acutatum and AFR and/or ARN when grown under the same conditions.

### V. Detection of a Modified Gene

Modification of a strawberry plant to insert a heterologous 163.0 or 163.26 gene, coding sequence, or fragment thereof using any of the described CRISPR constructs can be detected or confirmed by any means known in the art for detecting genetic modifications.

In some embodiments, a modification can be detected in a genomic DNA sample. Genomic DNA samples include, but are not limited to, genomic DNA isolated directly from a plant, cloned genomic DNA. or amplified genomic DNA.

Genetic analysis methods include, but are not limited to. polymerase chain reaction (PCR)-based detection methods (for example, TaqMan assays), microarray methods, mass spectrometry-based methods and/or nucleic acid sequencing methods, including whole genome sequencing. In some embodiments, the detection of genetic modification in a sample of DNA, RNA, or cDNA may be facilitated through the use of nucleic acid amplification methods. Such methods specifically increase the concentration of polynucleotides that span a target site, or include that site and sequences located either distal or proximal to it. Such amplified molecules can be readily detected by gel electrophoresis, fluorescence detection methods, or other means.

In some embodiments, insertion a heterologous 163.0 or 163.26 gene, coding sequence, or fragment thereof is detected by hybridization to allele-specific oligonucleotide (ASO) probes. ASO probes are disclosed in U.S. Pat. Nos. 5,468.613, 5,217,863. Single or multiple nucleotide variations in nucleic acid sequence can be detected in nucleic acids by a process in which the sequence containing the nucleotide variation is amplified, spotted on a membrane and treated with a labeled allele-specific oligonucleotide probe.

In some embodiments, insertion of a heterologous 163.0 or 163.26 gene, coding sequence, or fragment thereof is detected by probe ligation methods. Probe ligation methods disclosed in U.S. Pat. No. 5,800,944 where sequence of interest is amplified and hybridized to probes followed by ligation to detect a labeled part of the probe.

In some embodiments, microarrays can be used for detection of insertion of a heterologous 163.0 or 163.26 gene, coding sequence, or fragment thereof. For microarray detection, oligonucleotide probe sets are assembled in an overlapping fashion to represent a single sequence such that a difference in the target sequence at one point would result in partial probe hybridization (Borevitz et al., Genome Res. 13:513-523, 2003; Cui et al., Bioinformatics 21:3852-3858, 2005). Typing of target sequences by microarray-based methods is disclosed in U.S. Pat. Nos. 6,799,122: 6,913,879; and 6,996,476.

In some embodiments, insertion of a heterologous 163.0 or 163.26 gene, coding sequence, or fragment thereof can be directly identified or sequenced using nucleic acid sequencing technologies. Methods for nucleic acid sequencing are known in the art and include technologies provided by 454 Life Sciences (Branford, Conn.), Agencourt Bioscience (Beverly, Mass.), Applied Biosystems (Foster City, Calif), LI-COR Biosciences (Lincoln, Nebr.), NimbleGen Systems (Madison, Wis.), Illumina (San Diego, Calif), and VisiGen Biotechnologies (Houston. Tex.). Such nucleic acid sequencing technologies comprise formats such as parallel bead arrays, sequencing by ligation, capillary electrophoresis, electronic microchips, “biochips,” microarrays, parallel microchips, and single-molecule arrays.

In some embodiments, the presence of a FaRCa1 marker (e.g., 163.0 or 163.26 gene) in a plant may be detected through the use of a nucleotide probe. A probe may be, but is not limited to. nucleotide molecule. polynucleotide, oligonucleotide, DNA molecule, RNA molecule, PNA, UNA, locked nucleotide, or modified polynucleotide. Polynucleotides can be synthesized by any means known in the art. A probe may contain all or a portion of the nucleotide sequence of the genetic marker and optionally, one or more additional sequences. The one or more additional sequences can be contiguous nucleotide sequence from the plant genome, non-contiguous nucleotide sequence from the plant genome, or sequence that is not from the plant genome. Additional, contiguous nucleotide sequence can be “upstream” or “downstream” of the original marker. depending on whether the contiguous nucleotide sequence from the plant chromosome is on the 5′ or the 3′ side of the original marker, as conventionally understood. As is recognized by those of ordinary skill in the art, the process of obtaining additional, contiguous nucleotide sequence for inclusion in a marker may be repeated nearly indefinitely (limited only by the length of the chromosome), thereby identifying additional markers along the chromosome.

A polynucleotide probe may be labeled or unlabeled. A wide variety of techniques are readily available in the art for labeling a nucleotide probe. Nucleotide labels include, but are not limited to, radiolabeling, fluorophores, haptens, antibodies, antigens, enzymes, enzyme substrates, enzyme cofactors, and enzyme inhibitors. A label may provide a detectable signal by itself (e.g., a radiolabel or fluorophore) or in conjunction with other agents.

A probe may be an exact copy of a marker to be detected. A probe may also be a nucleic acid molecule comprising, or consisting of, a nucleotide sequence which is substantially identical to an inserted nucleic acid sequence. The term “substantially identical” may refer to nucleotide sequences that are more than 85% identical. For example, a substantially identical nucleotide sequence may be 86%, 87%, 88%, 89%, 90%, 91%, 92%, 93%, 94%, 95%, 96%, 97%, 98%, or 99% identical to the reference sequence.

A probe may also be a nucleic acid molecule that is “specifically hybridizable” or “specifically complementary” to an exact copy of the marker to be detected (“DNA target”). “Specifically hybridizable” and “specifically complementary” are terms that indicate a sufficient degree of complementarity such that stable and specific binding occurs between the nucleic acid molecule and the DNA target. A nucleic acid molecule need not be 100% complementary to its target sequence to be specifically hybridizable. A nucleic acid molecule is specifically hybridizable when there is a sufficient degree of complementarity to avoid non-specific binding of the nucleic acid to non-target sequences under conditions where specific binding is desired. Thus, an oligonucleotide probe is “specifically hybridizable” to a marker allele if stable and specific binding occurs between the oligonucleotide probe and the marker allele (e.g.. a SNP marker) under stringent hybridization conditions, but stable and specific binding does not occur between the oligonucleotide probe and the wild-type allele at the marker position.

In some embodiments, a probe comprises a pair of primers designed to produce an amplification product, wherein the amplification product is directly or indirectly determinative for the presence or absence of a AFR and/or ARN resistance marker.

### VI. Marker-Assisted Selection

Described are markers and methods for marker-assisted selection (MAS) of the FaRCa1 locus. Use of MAS in FaRCa1 breeding programs can be used to facilitate incorporation of the locus into diverse genetic backgrounds.

Described is a locus responsible for the AFR and/or ARN resistance in Fragaria plants (strawberries). The FaRCa1 locus maps to a 163-kb interval between 16,238,392 bp and 16,403,945 bp on chromosome 6.

FaRCa1 markers useful for genotyping (mapping, tracking, identifying, analyzing) a FaRCa1 locus in a Fragaria plants are described. In some embodiments, a FaRCa1 marker comprises a detectable genetic marker linked, closely linked, tightly linked, or extremely tightly to a FaRCa1 locus. In some embodiments, a FaRCa1 marker comprises a detectable genetic marker linked, closely linked, tightly linked, or extremely tightly linked to a genomic sequence encompassed by 16,238.392 bp and 16,403.945 bp on chromosome 6 of a Fragaria plant. In some embodiments, a FaRCa1 marker comprises a detectable genetic marker extremely tightly linked to a genomic sequence encompassed by 16,238,392 bp and 16,403,945 bp on chromosome 6 of a Fragaria plant. In some embodiments, a FaRCa1 marker is a detectable genetic marker linked, closely linked, tightly linked, or extremely tightly linked to one or more of: SEQ ID NO: 1 and/or SEQ ID NO: 3. In some embodiments, a FaRCa1 marker is a detectable genetic marker extremely tightly linked to one or more of: SEQ ID NO: 1 and/or SEQ ID NO: 3.

In some embodiments, a FaRCa1 marker comprises a PCR amplification product, a single nucleotide polymorphism (SNP), a restriction fragment length polymorphism (RFLP), an amplified fragment length polymorphism (AFLP), a simple sequence repeat (SSR), a simple sequence length polymorphism (SSLP), an insertion/deletion polymorphism (indel), a variable number tandem repeat (VNTRs), or a random amplified polymorphic DNA (RAPD) linked, closely linked, tightly linked, or extremely tightly linked to one or more of: SEQ ID NO: 1 and/or SEQ ID NO: 3.

With the described FaRCa1 markers, AFR and/or ARN resistant plants can be rapidly and efficiently identified. The identification of the FaRCa1 resistance locus can be used to aid in introgressing the AFR and/or ARN resistance trait into strawberry plants.

In some embodiments, the FaRCa1 markers may be used in marker-assisted selection to produce AFR and/or ARN resistant strawberry plants and/or introgress a AFR and/or ARN resistance trait from donor strawberry plants to recipient plants strawberry plants are described.

In some embodiments, the described FaRCa1 markers may be used in marker-assisted selection to transfer (introgress) segment(s) of DNA that contain one or more determinants of AFR and/or ARN resistance.

### VII. Brief Description of the Sequences

The nucleotide and amino acid sequences listed in the accompanying sequence listing are shown using standard letter abbreviations for nucleotide bases, and single-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding. it will be apparent that certain changes and modifications may be practiced within the scope of the appended claims.

### Examples

The following examples are provided to illustrate certain particular features and/or embodiments. These examples should not be construed to limit the disclosure to the particular features or embodiments described.

### Example 1. Materials and Methods

### A. Sample Collection and Sequencing

Young leaf tissue was covered with black plastic bags and kept in a greenhouse for 2 weeks. The etiolated leaf tissue was harvested for the DNA extraction. Leaves were frozen then submitted to DNA Link (Seoul, South Korea) for genomic DNA extraction and library preparation. The single-molecule real-time sequencing (SMRT) bell library was constructed using a PacBio DNA Template Prep Kit 1.0 (Pacific Biosciences). Quality and quantity of each library was checked using a 2100 Bioanalyzer (Agilent Technologies). The SMRT Bell-Polymerase complex was constructed using a PacBio Binding Kit 2.0 (Pacific Biosciences) based on the manufacturer's instructions. The complex was loaded onto five SMRT cells (Pacific Biosciences, Sequel SMRT Cell 1M v2) and sequenced using Sequel Sequencing Kit 2.1 (Pacific Biosciences, Sequel SMRT Cell 1M v2). For each SMRT cell, 1×600 min movies were captured using the Sequel sequencing platform (Pacific Biosciences) at DNA Link (Seoul, South Korea). The quality of Hifi was measured with LongQC (Fukasawa Y et al. LongQC: a quality control tool for third generation sequencing long read data. G3: Genes. Genomes, Genetics 10, 1193-1196 (2020)).

For transcriptome sequencing, six ‘Florida Brilliance’ tissues were used for construction of PacBio Iso-Seq libraries (Table 2). All ‘Florida Brilliance’ tissues were harvested from experimental fields at the Gulf Coast Research and Education Center (GCREC) in Wimauma, Florida between 14:00 and 16:00. Roughly 10 grams of 6 tissues were collected from 5 plants: flowers, green fruit, red fruit, crown, leaf, and root. All tissues were flash frozen at −80° C. after washing with water. Total RNA was extracted using Spectrum™ Plant Total RNA Kit (Sigma-Aldrich, St. Louis, MO. United States) according to manufacturer's instructions. To remove any traces of DNA removed with DNAse I (Invitrogen) and re-suspended in a total volume of 50 μl of RNase-free water. The six ‘Florida Brilliance’ tissues and RNA samples were submitted to DNA Link for preparation of Iso-Seq library. Pooled libraries were sequenced on SMRT-cells on the Sequel I platform.

### B. De Novo Genome Assembly and Validation

Overall genome characteristics including genome size and repetitive elements were estimated using PacBio HiFi data by K-mer spectrum distribution analysis for k=21 in KMC3 (Kokot M et al. KMC 3: counting and manipulating k-mer statistics. Bioinformatics 33, 2759-2761 (2017)) and GENOMESCOPE v 2.0 (Ranallo-Benavidez T R et al. GenomeScope 2.0 and Smudgeplot for reference-free profiling of polyploid genomes. Nature communications 11, 1-10 (2020)).

The HiFi and Hi-C read were used to produce a haplotype-phased assembly without sequencing of parents using Hifiasm (Cheng H et al. 2021). Hifiasm was run with the following command according to developer's recommendation for heterozygous crops: hifiasm -o<outputPrefix>-t<nThreads>-D10<Hifi-reads.fasta>--hl<Hi-C_reads1>--h2<Hi-C_reads2>. SALSA2, which is Hi-C-based scaffolding programs, was used for scaffolding contigs. Hi-C Reads were mapped by Arima-HiC mapping pipeline (https://github.com/ArimaGenomics/mapping_pipeline). After mapping Hi-C reads to each phased genome assembly using BWA v0.7.17 (Li H et al. Fast and accurate short read alignment with Burrows-Wheeler transform. Bioinformatics 25, 1754-1760 (2009a)) and sequence alignment map (SAM) format was converted to bed format using SAMtools (Li H et al. The sequence alignment/map format and SAMtools. Bioinformatics 25, 2078-2079 (2009b)) and BEDTools (Quinlan A R et al. BEDTools: a flexible suite of utilities for comparing genomic features. Bioinformatics 26, 841-842 (2010)) prior to SALSA2 scaffolding (https://github.com/marbl/SALSA; (Ghurye J et al. Integrating Hi-C links with assembly graphs for chromosome-scale assembly. PLoS computational biology 15, e1007273 (2019)), which was run with parameters -e GATC -m yes. Hi-C reads were mapped to chromosomes using HiC-Pro (Servant N et al. HiC-Pro: an optimized and flexible pipeline for Hi-C data processing. Genome biology 16, 1-11 (2015)) in order to assess the quality of assembly. The interaction matrix of whole chromosomes was visualized with heatmaps. Remaining contigs were scaffolded and oriented based on ‘15.89-25’ (Genome Database for Rosaceae) reference genome using Ragtag (Alonge M et al. RaGOO: fast and accurate reference-guided scaffolding of draft genomes. Genome biology 20, 1-17 (2019)).

### C. Scaffold Validation Using a Genetic Linkage Map

A high-density genetic map was developed using a total of 169 F1 individuals from a cross between ‘Florida Brilliance’ and 16.33-8. Axiom™ IStraw90 Genotyping Array were used to genotype all 169 F1 individuals. Markers were filtered to have<5% missing data and fit segregation ratios of 1:1 and 1:2:1 (α=0.05). Marker genotype calls were recoded to fit Joinmap 4.1 linkage mapping requirement. For example, markers with paternal segregation (AA×AB or BB×AB) coded as “nn×np”; markers with maternal segregation (AB×AA or AB×BB) coded as “lm×ll”; and markers segregating in both parents (AB×AB) coded as “hk×hk.” Mapping was conducted in an iterative process using the maximum likelihood algorithm in JoinMap 4.1 with default settings. After each round of mapping, a graphical genotyping approach was applied to identify singletons to fix the marker order and regions with low marker density or gaps caused by segregation distortion. The genetic linkage map of ‘Florida Brilliance’ consisting of 10,269 SNP markers was used to validate the scaffolds from the FaFB1 whole genome assembly. SNP probes sequences used in the construction of linkage maps were mapped to the FaFB1 assembly sequence using blastn procedure (Camacho C et al. BLAST+: architecture and applications. BMC bioinformatics 10, 1-9 (2009)). Alignments were filtered to retain markers if they matched to unique sequence position in the FaFB1 phased genome assembly and with a maximum of 2 mismatches in the second-best hit. The alignments were queried to detect problematic scaffolds mapped with SNP probes from different LGs. The number of scaffolds with SNP probes mapped from different LGs was used as a metric in the quality assessment of FaFB1 assembly.

### D. Assembly Quality Evaluation

Genome assembly statistics were calculated using QUAST version 5.0.266. Merqury version 1.3 were used to measure assembly consensus quality value (QV), evaluates assembly based on efficient K-mer set operations (Rhie A et al. Merqury: reference-free quality, completeness, and phasing assessment for genome assemblies. Genome biology 21, 1-27 (2020)). The completeness of the haploid assemblies and protein-coding gene annotations were assessed with the BUSCO database (Simao F A et al. BUSCO: assessing genome assembly and annotation completeness with single-copy orthologs. Bioinformatics 31, 3210-3212 (2015)). The scaffolds were inspected on the Hi-C contact map. Hi-C reads were trimmed with Homer (Heinz S et al. Simple combinations of lineage-determining transcription factors prime cis-regulatory elements required for macrophage and B cell identities. Molecular cell 38, 576-589 (2010)) and mapped to the both haploid assembly using HiC-Pro version 3.0 (Servant N et al. 2015) and visualized in Juicebox version 1.11 (Durand N C et al. Juicebox provides a visualization system for Hi-C contact maps with unlimited zoom. Cell systems 3, 99-101 (2016)). LAI (10) for each sub-genome was calculated using LTR-retriever (Ou S et al. LTR_retriever: a highly accurate and sensitive program for identification of long terminal repeat retrotransposons. Plant physiology 176, 1410-1422 (2018)) along with whole-genome TE-annotations and intact LTR retrotransposons identified by EDTA (Ou S et al. Benchmarking transposable element annotation methods for creation of a streamlined, comprehensive pipeline. Genome biology 20, 1-18 (2019)).

### E. Repetitive Annotation

Transposable elements (TEs) were annotated using EDTA v1.9.6 with default parameters (Su W et al. A tutorial of EDTA: extensive de novo TE annotator. Plant Transposable Elements, 55-67 (2021)). The TE annotation library was generated by EDTA in a separate run. TE regions of both haploid assemblies were masked by ReapeatMasker v4.1.1 provided with the repeat library. Simple sequence repeats (SSRs) or microsatellites were mined using SSR Finder (Humann J L et al. “Structural and functional annotation of eukaryotic genomes with GenSAS” in Gene prediction. (Springer, 2019), pp. 29-51) on Genome Sequence Annotation Server v6.0 (GenSAS). Telomeric repeats were annotated using BIOSERF (Somanathan I et al. A bioinformatics approach to identify telomere sequences. Biotechniques 65, 20-25 (2018)).

### F. Transcriptome Assembly and Gene Annotation

To increase the accuracy of gene annotation, we generated a transcriptome assembly containing possible sets of transcripts from ‘Florida Brilliance’ and F.×ananassa expression data publicly available. Sixty-seven octoploid strawberry RNA-Seq libraries were downloaded from NCBI sequence read archive (SRA). Octoploid strawberry ‘Royal Royce’ Iso-Seq reads were trimmed using Trimmomatic version 0.39 and mapped to two phased-assemblies using HISAT v2.2.1 (Kim D et al. Graph-based genome alignment and genotyping with HISAT2 and HISAT-genotype. Nature biotechnology 37, 907-915 (2019)) with default parameters. The ‘Florida Brilliance’ Iso-Seq reads were aligned to the assemblies using minimap v2.2.1 (Li H Minimap2: pairwise alignment for nucleotide sequences. Bioinformatics 34, 3094-3100 (2018)). Reads alignment were converted to Binary alignment map (BAM) format with samtools. Reference-guided transcriptome assembly was performed using StringTie v2.1.4 (Kovaka S et al. Transcriptome assembly from long-read RNA-seq alignments with StringTie2. Genome biology 20, 1-13 (2019)) with the Iso-Seq and RNA-Seq alignments as input. StringTie2 was run with default parameters for RNA-Seq alignment, and with the addition of long read (-L) mode for Iso-Seq alignments. Mikado v2 (Venturini et al. Leveraging multiple transcriptome assembly methods for improved gene structure annotation. GigaScience 7, giy093 (2018)) was used to generate a non-redundant set of transcript assemblies with best-scoring transcript evidence at each locus. Match scores were measured for all transcriptome assemblies against the UniProt protein database using BLASTX. TransDecoder v5.5.0 (https://github.com/TransDecoder/TransDecoder) were used to predict the best six-frame translations of the transcriptome assemblies from StringTie2, then splice junctions for all merged RNA alignments were predicted with Portcullis v1.2.2 (Mapleson D et al. Efficient and accurate detection of splice junctions from RNA-seq with Portcullis. GigaScience 7. giy 131 (2018)). The Mikado scoring for any transcript assemblies derived from Iso-Seq alignments was modified over RNA-Seq alignments. TransDecoder was used to filter non-redundant, polished transcripts generated by Mikado to obtain best ORF scores.

“Florida Brilliance” genome assembly was annotated using GenSAS v6.0 (Humann J L et al. 2019). Non-redundant transcript assemblies derived from Mikado2 pipeline were provided as EST evidence. Transcripts were aligned to combined phased-1 and phased-2 assemblies using BlastN (Camacho C et al. 2009). The alignments were combined with results of gene prediction using AUGUSTUS v3.1.1 (Stanke M et al. AUGUSTUS: ab initio prediction of alternative transcripts. Nucleic acids research 34, W435-W439 (2006)) to generate an official gene set and identify predicted transcripts within each masked assembly (EVidenceModeler) (Haas B J et al. De novo transcript sequence reconstruction from RNA-seq using the Trinity platform for reference generation and analysis. Nature protocols 8, 1494-1512 (2013); and Haas B J et al. Automated eukaryotic gene structure annotation using EVidenceModeler and the Program to Assemble Spliced Alignments. Genome biology 9. 1-22 (2008)). Function of predicted transcripts were annotated based on alignment using BlastP v2.2.28 to the UniProtKB database (Boutet E et al. “Uniprotkb/swiss-prot” in Plant bioinformatics. (Springer, 2007), pp. 89-112).

### G. Collinearity and Synteny

Proteins of diploid strawberry progenitor F. vesca were collected for all-against-all alignments to predicted proteins for Octoploid strawberry ‘Florida Brilliance’. These alignments were passed to MCScanX to identify synteny blocks (Wang Y et al. MCScanX: a toolkit for detection and evolutionary analysis of gene synteny and collinearity. Nucleic acids research 40, e49-e49 (2012)). DNA level synteny between F. vesca. F.×ananassa. F. chiloensis and two phased genome assemblies of ‘Florida Brilliance’ were all plotted using D-GENIES (Cabanettes F et al. D-GENIES: dot plot large genomes in an interactive, efficient, and simple way. PeerJ 6, e4958 (2018)) with default parameters after aligning with minimap2.

### H. Availability of Supporting Data and Materials

The chromosome-level goose genome assembly sequence is available at NCBI GenBank through accession No. SAMN31220431; the Hi-C data are available through the NCBI SRA database (submission numbers SUB12134978 (PRJNA888562) and SUB12134925). The PacBio long-read sequencing data have been deposited in the NCBI SRA. The transcriptome data are available through NCBI. The chromosome-level goose genome assembly, annotation files, and other supporting data are available via genome database for rosaceae (GDR).

### I. Bacterial Artificial Clone (BAC) Library Screening

Bacterial artificial clone libraries for genotypes 11.77-96. ‘Florida Brilliance’ and 14.101-225 were previously constructed by Amplicon Express (https://ampliconexpress.com/) using restriction enzymes HindIII and BamHI. Approximately 32,000 inserts were cloned in vector pCC1 BAC (EPICENTRE, Madison, WI, USA) and transformed in the host E. co/i cells Phage Restraint DH10B per genotype. The average insert size obtained with BamHI digestion was 95 kb, 100 kb and 100 kb for 11.77-96, ‘Florida Brilliance’ and 14.101-225, respectively. The average insert obtained with HindIII digestion was 100 kb, 115 kb and 100 kb for 11.77-96, ‘Florida Brilliance’ and 14.101-225, respectively. Each restriction enzyme digestion produced ˜16,000 inserts for each genotype.

Screening of six BAC clone libraries was done using the Amplicon Express method (https://ampliconexpress.com/). The matrix pooling and superpooling system was based on two separate rounds of PCR on extracted DNA from independently grown, then separately pooled, BAC clones. The Round I PCR was performed on the superpool collection plate. The Round II PCR was performed on the matrix pools corresponding for the specific Superpool identified in Round I PCR. Round II PCR required 21 PCR experiments plus controls for each positive hit obtained from Round I PCR. The results from Round II PCR allows the identification of the plate and well position of a single positive hit using the key provided by Amplicon Express. This matrix system reduces the number of PCR reactions by 50%.

### J. Primer Development for BAC Screening

To identify BAC clones that contained susceptible and resistant haplotypes in FaRCa1 in Fvb6-3, probe sequences for each of the three SNPs and one InDel spanning the FaRCa1 region (AX-89896208, 9-bp InDel close to AX-89838986, AX-89808208 and AX-89838962) were aligned to the four chromosomes Fvb 6-1, 6-2, 6-3, and 6-4 from the octoploid ‘Camarosa’ genome using Geneious R10.2 software (Geneious). Approximately 5,000 bp around each SNP were extracted from each chromosome. The extracted sequences for each SNP from all or most chromosomes Fvb6 were aligned. Primer design was performed based on this alignment using PrimerQuest software from Integrated DNA Technologies, Inc. (Primerquest).

Primer design parameters were customized by setting the primer temperature minimum to 58° C. optimum to 60° C., and maximum to 62° C. The amplicon size was selected to be between 600 and 800 bp. The ideal primer size was 22 bp, with an amplicon size of 700 bp. If no primers could be found within these parameters, the amplicon size was increased up to 900 bp. Three forward primers and three reverse primers (Table 11) for each SNP were tested. Best primers for each SNP were those flanking SNPs that aligned to all or most of Fvb 6 subgenomes and showed slight background and strong brightness when visualized in 1% agarose gels. The positive PCR control primers were used according to the provider's recommendation (Amplicon Express, https://ampliconexpress.com/) (Table 11).

### K. Superpool Collection Screening (Round I PCR)

The superpool collection screening was done using six pairs of primers located in the FaRCa1 region and their homologous region in Fvb 6-1, 6-2, and 6-4 from the ‘Camarosa’ genome. The 5 pl PCR reactions were composed of 2.5 pl of 2×AccuStart II PCR ToughMix master mix (Quantabio, Beverly, MA), 0.25 μl of 10 μM forward and 0.25 μl of 10 M reverse primer. 1 μl of BAC clone DNA from superpool collection plates, and 1 μl deionized H₂O. The reactions were carried out in a 384-well PCR plate in a LightCycler 480 II Instrument (Roche, Basel, Switzerland). Conditions of the PCR were as follows: initial denaturation at 94° C. for 120 s, 35 cycles of denaturation at 94° C. for 30 s, annealing at 60° C. for 30 s, and extension at 72° C. for 60 s. After amplification, the PCR product was denatured at 72° C. for 600 s and cooled to 4° C. Positive and negative results were included according to manufacturer indications (Amplicon Express, https://ampliconexpress.com/). Products obtained from Round T PCR reactions were visualized in 1% agarose gel stained with SYBR® safe DNA gel stain. Product size was checked with a 1 kb DNA ladder (BioLabs, N3232S). Bright clear bands in the gel were chosen for subsequent PCR reactions in the matrix pool screening (Round II PCR).

### L. Matrix Pool Screening (Round II PCR)

The matrix pool screening was done based on positive hits obtained in the superpool screening using the same six primers used in Round I PCR. The 5 μl PCR reactions were composed of 2.5 μl of 2×AccuStart II PCR ToughMix master mix (Quantabio, Beverly, MA), 0.25 μl of 10×LCGreen Plus Melting Dye (BioFire Defense, Salt Lake City, UT), 0.25 μl of 10 μM forward and 0.25 μl of 10 μM reverse primer, 1 μl of BAC clone DNA from Matrix pool collection plates, and 0.75 μl of deionized H₂O. The reactions were carried out in a 384-well PCR plate in a LightCycler 480 II Instrument (Roche, Basel, Switzerland). Conditions of PCR were as follows: initial denaturation at 94° C. for 120 s. 35 cycles of denaturation at 94° C. for 30 s, annealing at 60° C. for 30 s, and extension at 72° C. for 60 s. After amplification, the PCR product was denatured at 72° C. for 600 s and cooled to 4° C. Positive and negative results were included according to manufacturer's indications (Amplicon Express, https://ampliconexpress.com/).

Products obtained from Round II PCR reactions were analyzed by HRM with these conditions: 95° C. for 60 s, 40° C. for 60 s, 65° C. with 1° C. per second with 25 acquisitions per degree until 95° C. and cooled to 4° C. per 30 s. Melting curve data were collected and analyzed using the HRM software Melt Curve Genotyping and Gene Scanning available on the Roche LightCycler 480 II system. Samples with peaks regardless of size and shape were considered positive hits meaning potential clones with inserts containing the FaRCa1 region in four subgenomes. The matrix pool keys were used to find the exact location of BAC clones including plate, row, and column of each positive hit from each of the libraries screened.

### M. Plasmid DNA Extraction of Selected BAC Clones

After identification of the position of 139 BAC clones in the 348-well plates from the six libraries, BAC clones were revived in LB agar plates containing 20 μg/mL of chloramphenicol. To extract plasmid DNA, BAC clones were transferred from LB agar plates to 3 ml of liquid LB medium containing 20 μg/mL of chloramphenicol. Tubes were incubated for 17 h at 37° C. and 250 rpm. Plasmid DNA was extracted using the Zyppy Plasmid Miniprep kit (Zymo Research, Irvine, CA). DNA concentration was measured using a Nanodrop 8000 Spectrophotometer (Thermo Scientific). Plasmid DNA was diluted to 50 ng/gl.

### N. BAC Clones Screening Using Subgenome-Specific Primers

The subgenome-specific HRM marker for an InDel designed for MAS was used to screen the 139 BAC clones and differentiate BAC clones containing resistant and susceptible alleles of FaRCa1 in Fvb 6-3. The 5 μl PCR reactions were composed of 2.5 μl of 2×AccuStart II PCR ToughMix master mix (Quantabio, Beverly, MA), 0.25 μl of 10×LCGreen Plus Melting Dye (BioFire Defense, Salt Lake City, UT), 0.5 μl of 5 μM forward and 0.5 μl of 5 μM reverse primer, 1 μl of plasmid DNA, and 0.25 μl of deionized H₂O. The reactions were carried out in a 384-well PCR plate in a LightCycler 480 II Instrument (Roche, Basel, Switzerland). The PCR conditions were as follows: initial denaturation at 95° C. for 5 min. 55 cycles of denaturation at 95° C. for 15 s, annealing at 60° C. for 15 s. and extension at 72° C. for 15 s. After amplification, the PCR product was denatured at 72° C. for 7 min and cooled to 4° C. HRM conditions were: 95° C. for 60 s, 40° C. for 60 s, 55° C. with 1° C. per second with 25 acquisitions per degree until 95° C. Melting curve data were collected and analyzed using the HRM software Melt Curve Genotyping and Gene Scanning available on the Roche LightCycler 480 II system. Samples that showed a peak were classified as susceptible or resistant BAC clones according to HRM curves previously obtained for the InDel primer.

### O. BAC End-Sequencing and NGS Sequencing

Plasmid DNA from BAC clones that showed peaks with InDel HRM marker were extracted using QIAGENC® Plasmid Mini Kit (QIAGEN, Valencia, CA). DNA concentrations and quality were obtained using Nanodrop 8000 Spectrophotometer (Thermo Scientific). High-quality DNA samples were sent for Sanger end-sequencing using T7 and M13 reverse primers (GENEWIZ, South Plainfield, NJ, USA). BAC clone sequences obtained were blasted to the ‘Camarosa’ genome3 using Geneious R10.2 software (Geneious). DNA from BAC clones that showed specific complementarity to Fvb6-3 were re-extracted using the QIAGEN® Plasmid Midi Kit (QIAGEN, Valencia, CA) for next-generation sequencing. DNA quantification was assessed with Spectrophotometer (Thermo Scientific) and Qubit™ 1×dsDNA high sensitivity (HS) assay kits. Qualified DNA was cut into fragments by restriction enzymes (NOVOGENE, Chula Vista, CA, USA). DNA libraries were constructed by end repairing, adding A nucleotides to tails, purification, and PCR amplification. Qualified libraries were sequenced by Illumina high-throughput sequencer with paired-end sequencing using the Illumina hPE150. Q30>80% (NOVOGENE, Chula Vista, CA, USA). Raw sequenced reads were filtered by eliminating low quality reads and adapters.

### P. BAC Clone Assembly, Annotation, and Gene Candidate Detection

The 5.94-G filtered sequencing data obtained from four BAC clones were Illumina de novo assembled independently (unpublished). A contig-level ‘Florida Brilliance’ assembly using 20× PacBio data was preliminary assembled using Canu assembler 144 (correcter error rate=0.105, cor out coverage=200) (unpublished). ‘Florida Brilliance’ PacBio contigs were used to 1) scaffold the disconnected resistant contigs from the de novo BACs assemblies, 2) re-examine the BAC de novo assemblies, and 3) compare sequences from the susceptible and resistance alleles. Previously assembled ‘Florida Brilliance’ HiFi data (unpublished) was also used to confirm resistant and susceptible allele sequences from BAC de novo assemblies.

‘Camarosa’ annotated genomes 3,143 were used to uncover genes in the FaRCa1 region using available information in the Genome Database for Rosaceae (GDR)147. Genes were visualized using the genome browser JBrowse 1.16.4148 from the GDR rosaceae webpage. Sequences of genes obtained from the ‘Camarosa’ annotation 143 were used as BLAST query in NCBI data sets. Susceptible and resistant haplotypes for potential resistance genes were compared to detect functional polymorphisms. Polymorphisms in the genes located in FaRCa1 region were confirmed using the ‘Florida Brilliance’ HiFi assemblies (unpublished). Also, RNAseq data from different tissues of ‘Florida Brilliance’ including crown, leaf, root, flower. green fruit, and red fruit were aligned to candidate genes associated with disease resistance in the FaRCa1 region to find transcript sequences and confirm start and stop codons. InterPro 87.0149 was used to classify proteins into families and to predict domains and important sites.

### Q. RNAi Vector Construction

Hairpin structures were designed for two genes based on their potential to be responsible of C. acutatum resistance in strawberry using the CLC Genomic Workbench 11. Genes named as augustus masked-Fvb6-3-processed-gene-163.0 and maker-Fvb6-3-augustus-gene-163.26, according to the ‘Camarosa’ genome annotation v1.03, or as FxaC_22g29230 and FxaC_22g29220 according to the ‘Camarosa’ genome annotation v1.a2143, were chosen to continue with experimentation and thus, renamed to gene 163.0 and 163.26, respectively. Hairpin inserts are composed of a 300 bp stem, 100 bp loop, and aattB sites for Gateway® cloning (Table 14). The 300 bp steam in each of the inserts was complementary to the first coding DNA sequence (CDS) region of gene 163.0 and 163.26 according to the ‘Camarosa’ annotation v1.03. The 758-bp fragments were synthetized using GeneArt™ gene synthesis (Thermo Fisher Scientific). The inserts were individually ligated upstream of the E. coli sites to the vector pMK-QR containing the kanamycin selection gene.

Vector pMK-QR containing the hairpin fragments were used in the Gateway® protocol for 163.0 and 163.26, independently. Hairpins were cloned into the Gateway®-pDONR™/Zeo vector (Thermo Fisher Scientific, Waltham, MA, USA) using standard procedures (Table 14). The insert identity was confirmed by PCR of two colonies for each target gene. After checking, the fragments were inserted into the silencing RNAi Gateway® vector pK7GWIWG2(I) and the transformants were confirmed by PCR and by sequencing. Vectors containing the constructs and empty vector were separately inserted into Agrobacterium tumefaciens strain EHA105 by using an adapted freeze-thaw method. The transformed cells were tested using PCR for the presence or absence of RNAi constructs. Transformed bacterial cells were kept in a solution of equal amount of LB and 50% glycerol solution at −80° C.

The 20 μl PCR reactions for confirmation of RNAi inserts were composed of 2 μl of 10×BioReady Buffer, 0.1 μl of rTaq, 0.4 μl of 10 mM dNTPs, 1 μl of 5 μM forward and 1 μl of 5 μM reverse primer, 5.5 μl of plasmid DNA, and 10 μl of deionized H₂O. The reactions were carried out in 8-tube PCR strips in a ProFlex PCR system (Applied biosystems by Life Technologies). Conditions of PCR were as follows: initial denaturation at 94° C. for 3 min, 35 cycles of denaturation at 94° C. for 30 s, annealing at 58° C. for 30 s, and extension at 72° C. for 60 s. After amplification, the PCR product was denatured at 72° C. for 5 min and cooled to 4° C. Products were visualized in 1% agarose gels stained with SYBRCR safe DNA gel stain. Product size was checked with a 100 bp ladder.

### R. Agrobacterium-Mediated Fruit Transformation

Agrobacterium-mediated fruit transformation was performed as described by Pi et al. and Zhao et al. with modifications, and was repeated five times during the 2020-21 strawberry season at the UF Gulf Coast Research and Education Center (GCREC). UF accessions with variable phenotype and genotype for C. acutatum resistance were used. Breeding selection 17.20-51 is homozygous resistant (Ca1Ca1), ‘Florida Brilliance’ is heterozygous resistant (Ca1 ca1) and 16.74-68 is homozygous susceptible (ca1 ca1). These three UF genotypes were transformed with Agrobacterium tumefaciens strain EHA105 applying three treatments: (a) A. tumefaciens transformed with empty vector; (b) A. tumefaciens transformed with RNAi for gene 163.0; and (c)A. tumefaciens transformed with RNAi for gene 163.26.

Transformed Agrobacterium tumefaciens were revived by transferring cells from the 1:1, water: 50% glycerol solution with sterilized toothpicks in 5 ml of Luria Broth (LB) medium with rifampicin (15 mg/L) and spectinomycin (100 mg/L) two days before fruit agroinfiltration. Revived A. tumefaciens was transferred to new 5 ml of LB medium and was allowed to grow for 24 h at 28° C. and 230 rpm. Concentrations of bacterial cells were measured with the value OD600 using Biomate 3S UV-visible spectrophotometer (Thermo Scientific). When cultures reached OD600 of about 1, 1.25 ml, each was transferred to 250 ml of LB in a flask. A. tumefaciens grew for about 18 h at 28° C., 230 rpm until reaching an OD600 value of about 1. Bacterial cells were collected by centrifuging culture in 50-ml tubes at 40,000 rpm for 20 min. Agrobacterium cells were resuspended in 600 ml of activation buffer (4.43 gr/L Murashige and Skoog medium, 10 mM MgCl2, 200 M acetosyringone, 10 mM 2-(N-morpholino) ethane sulfonic acid, pH=5.8). Concentration was fixed at OD600=0.5 for Rep 1 and 0.8 for Rep 2, 3, 4, and 5. Activated solution was kept in an orbital shaker Cole-Parmer for three hours at room temperature at 100 rpm.

Ninety green fruits of each genotype were harvested in the early morning. Healthy uniform fruit at similar development stage were preferred 146. Pedicels were carefully removed to avoid contamination during the experiment. Fruit was disinfected by immersion in a 0.7% solution of sodium hypochlorite (bleach) for six minutes. Fruit was rinsed for four times with sterilized water, distributed in disinfected containers, and placed in the hood for 20 minutes to dry. Dried fruits were classified, arranged by size, and evenly distributed in disinfected egg shelves inside a disinfected plastic box33. Agroinfiltration was done by injecting the activated solution for each of the constructs to the green fruit using 5 ml—syringes until saturation 146. Sterilized water was poured in the plastic containers to maintain humidity. Agroinfiltrated fruit were kept under 16 hours of daylight and 8 h of dark conditions at room temperature.

At day five to six, tissue for RNA extraction was collected from 10 fruits from each treatment. Fruit was cut horizontally removing the tip and the pedicel resulting in a 1-cm disc. Internal area of the disc was also discarded. Tissue of the 1 cm-disc was cut in small pieces and immediately frozen with liquid nitrogen. Pieces from all the 10 fruits were combined and collected in a 50-ml tube. Three 50-ml tubes for each agroinfiltration replication were stored at −80° C. before processing.

S. C. acutatum Inoculation

Agroinfiltrated fruit were inoculated with C. acutatum five days after agroinfiltration. Inoculum was produced by growing the three isolates of C. acutatum (C. nymphaeae) 02-163. 02-179 and 03-32 separately on potato-dextrose agar (PDA) at room temperature with constant light for seven days until sporulation. Plates were flooded with sterile-distilled water when growth turned orange and reached near the border of the plate. A spore suspension for each isolate was passed through cheesecloth to remove dislodged mycelia. A final suspension was adjusted to 1×10⁶ conidia/ml by combining equal concentrations of each isolate. Fruits were inoculated with a single 20-μl drop of C. acutatum inoculum in the hood.

### T. Phenotype Scoring and Analysis

Pictures were taken daily after C. acutatum inoculation for 5-6 days using an EOS Rebel T5 camera (Canon). At day 12-13 after agroinfiltration, agroinfiltrated inoculated fruit were scored externally and internally. Symptomatic fruit were cut along the middle of the lesion to expose the internal symptoms before pictures were taken on the last day of the experiment.

External and internal symptoms were analyzed using the software ImageJ 1.53e. Area of fruit, area of external symptoms, area of halves of cut fruit, and area of internal symptom were obtained. Agroinfiltration. inoculation and phenotype scoring were performed five times during the strawberry season 2020-21. Agroinfiltration experiments for each replication began at 12/31/2020, 1/21/2021, 2/15/2021, 3/9/2021 and 3/30/2021. Comparison of means and separation of means was performed using two-way analysis of variance (ANOVA) and the least significant difference (LSD) test in R software (R Core Team 2000).

U. Gene Expression Profiling After C acutatum Inoculation

Forty-five white or pink-spotted white fruit from each genotype were harvested in the early morning. Healthy uniform fruit at similar development stages were preferred. Fruit disinfection, classification, arrangement, and C. acutatum inoculation were done as previously described in the gene silencing experiment. Three biological replications composed of tissue from three fruit each were taken every day at the same time at five time points: 0, 24, 48, 72 and 96 h post inoculation (hpi) for each genotype. Samples collected at 0 hpi were taken from non-inoculated fruit. Samples collected at 24, 48, 72 and 96 hpi were obtained by measuring 2-cm squares around the lesion caused by the C. acutatum inoculation. Fruit tissue was cut in small pieces and immediately frozen using liquid nitrogen. Samples were kept at −80° C.

### V. RNA Extraction and Quantification

Fruit tissue was homogenized to a fine powder by using Tissue Lvser II (Qiageni)) for 1 min. RNA was extracted by using the Spectrum™ Plant Total RNA Kit (Sigma-Aldrich, MO, USA) as recommended by the manufacturer. RNA concentration and quality were assessed using Nanodrop 8000 Spectrophotometer (Thermo Scientific) and Qubit™ RNA broad range (BR) assay kits. DNA contaminants in the RNA samples were treated with Deoxyribonuclease (DNase) I, amplification grade (Invitrogen, MA, USA) according to manufacturer instructions.

W. cDNA Synthesis and Gene Expression Analysis Using Real-Time Quantitative Analysis

cDNA was synthesized in a 20 pd reaction composed by 4 μl of LunaScript® RT SuperMix Kit (New England, MA, USA) and 16 μl containing 500 ng of total RNA. The cDNA synthesis reactions were conducted in a 96-well PCR plate in a ProFlex PCR system (Applied biosystems by Life Technologies). cDNA synthesis conditions were primer annealing at 25° C. for 2 min, cDNA synthesis at 55° C. for 10 min and heat inactivation at 95° C. for 1 min. Each cDNA sample was diluted 5×and 0.25×before being added to the quantitative real-time PCR (qRT-PCR).

The qRT-PCR experiment was performed in triplicates of 5 μl reactions containing 2.5 μl of 2×EvaGreen® based Forget-Me-Not™ qPCR (Biotium, CA. USA) master mix, 0.4 μl of 0.5 μM forward primer solution, 0.4 μl of 0.5 μM reverse primer solution, I pl of diluted cDNA and 0.7 μl of DI H2O. The target genes were FaGapDH2, 163.0 and 163.26. The 5×cDNA dilution was used for the PCR reactions containing primers for the housekeeping gene FaGapDH2 and the 0.25×dilution, for PCR reactions with primers for genes 163.0 and 163.26 (Table 15). The reactions were carried out in a 384-well PCR plate in a LightCyclerX480 II Instrument (Roche, Switzerland) for both PCR and HRM. Conditions of PCR were: preincubation at 95° C. for 5 min, initial denaturation at 95° C. for 20 s, 40 cycles denaturation at 95° C. for 20 s, annealing at 62° C. for 10 s and extension at 72° C. for 10 s. HRM conditions were 95° C. for 5 s, 65° C. for 1 min and 97° C. continuous. Finally, the PCR product was cooled to 40° C. for 30 s to allow heteroduplex formation. The raw cycle threshold values (Cp) from qRT-PCR runs were used to calculate relative expression using the ΔCt method.

X. Comparative Genomic Analysis with All Subgenomes

Resistant haplotypes obtained from SL001 and SL002 alignments were used to run comparative genomic analysis with homoeologous FaRCa1 regions in subgenomes Fvb 6-1, 6-2, 6-3 and 6-4 from ‘Camarosa’ using the progressiveMauve algorithm152 in Geneious Prime® 2022.0.1

### Example 2. Octoploid Strawberry Genomic and Transcriptomic Datasets

The details of read data used are provide in Table 1. With five single-molecular real-time cells in PacBio Sequel II platform, 144.1 Gb of sequences were generated in 9.1M reads. The average read length was 15.834.8 bp. The Hi-C data was generated on Novaseq6000 and contained 86.5 Gb of sequences in 286 M paired-end reads with an average length of 151 bp. In total, 6.56 Gb of ‘Florida Brilliance’ Iso-Seq sequence data with an average length of 2,558 bp were generated. Among the iso-seq sequences, when restricted to HiFi (QV>20), 45,577 to 56.707 reads with an average length of about 2,500 bp were obtained from 6 tissues (Table 2). In total, 12.1 Gb of ‘Royal Royce’ Iso-Seq sequence data (Hardigan M A et al. 2021) and 511 Gb of short read Read-seq were used as input for transcriptome assembly and gene annotation.

TABLE 1
Libraries sequenced and used in assembly with accession numbers.

| | | No. of | Number | Average of | Number |
|---|---|---|---|---|---|
| Library | | sequencing | of reads | read length | of bases |
| type | Tissue | runs | (#) | (bp) | (Gb) |
| Pacbio | Leaf | 5 | 9,101,332 | 15,834.8 | 144.10 |
| HiFi | | | | | |
| Hi-C | Leaf | 2 | 572,765,774 | 151 | 86.50 |
| Iso-Seq | Leaf, crown, flower, green | 1 | 2,567,240 | 2,558 | 6.56 |
| | fruit, red fruit, root | | | | |

TABLE 2
Statistics of HiFi reads (QV ≥ 20) generated from iso-seq
sequences from 6 tissues of ‘Florida Brilliance’.

| | | Number | Average |
|---|---|---|---|
| Tissue | Yield (bp) | of reads | length |
| Leaf | 125,573,371 | 50,973 | 2,463.5 |
| Crown | 138,458,257 | 56,707 | 2,441.6 |
| Flower | 120,850,566 | 51,203 | 2,360.2 |
| Green fruit | 132,446,690 | 54,049 | 2,450.5 |
| Red fruit | 132,761,812 | 49,638 | 2,674.6 |
| Root | 116,103,170 | 45,577 | 2,547.4 |
| Total | 766,193,866 | 308,147 | |
| Average | | | 2,486.5 |

### Example 3. Phased Octoploid Genome Assembly

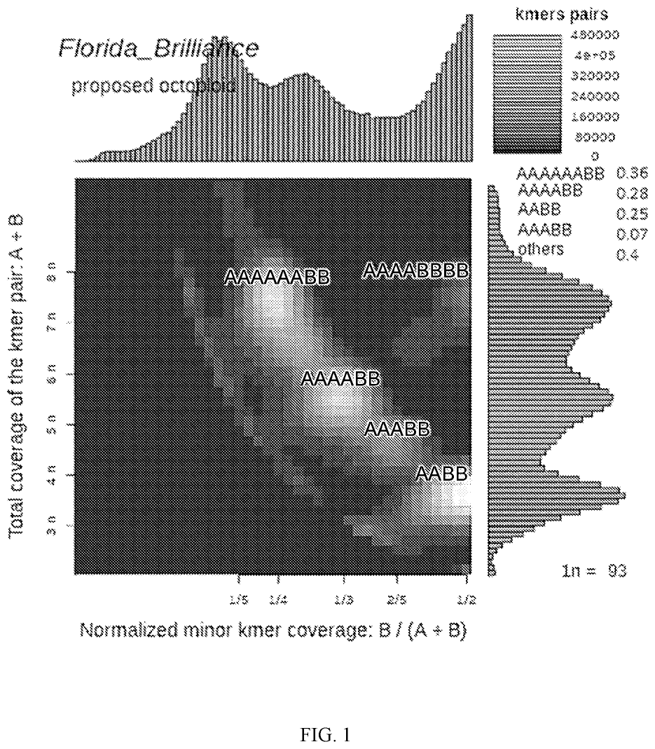

The octoploid strawberry genome of ‘Florida Brilliance’ was assembled using HiFi and Hi-C reads. Smudgeplot based on K-mer analysis using HiFi reads showed that ‘Florida Brilliance’ is allo-octoploid species with genome structure of ‘AAAAAABB’ (FIG. 1). The phase-1 assembly contained 3.716 contigs with an N50 of 23.7 Mb, and the phased-2 assembly contained 1,226 contigs with an N50 of 26.7 Mb (Table 3). Fifteen contigs accounted for 50% of phased-2 assembly, indicating that one contig corresponds to a chromosome (Table 3). In addition, largest contig size in phased-1 and phased-2 genome assemblies were over 36 Mb (Table 2). Before scaffolding, the Benchmarking Universal Single-Copy Orthologs (BUSCO) scores were 99.2% in phased-1 assembly and 99.1% in phased-2 assembly, indicating high-quality of initial assembly (Table 3). Comparison of the full assembly to whole genome sequencing HiFi reads of FaFB2 using Merqury showed very high base accuracy (QV>69.8), indicating 99.99999% of HiFi reads were detected on the combined phased-1 and 2 contigs (Table 3). The final assembly contained 99.1% complete gene models with a majority (96.6%) of the duplicated complete gene models in both phased-1 and phased-2 genome assembly (Table 3).

TABLE 3
Statistics of the ‘Florida Brilliance’
genome assembly and annotation.

| | Phased-1 | Phased-2 |
|---|---|---|
| Assembly Metrics | assembly | assembly |
| Draft assembly | | |
| Number of contigs | 3,716 | 1,226 |
| Number of contigs (≥50 kb) | 750 | 618 |
| Length of largest contig (Mb) | 34.6 | 34.8 |
| Assembled genome size (Mb) | 866 | 839.13 |
| GC content (%) | 40.67 | 39.84 |
| Length of contig N50 (Mb) | 23.72 | 26.65 |
| Length of contig N75 (Mb) | 12.38 | 17.30 |
| L50 | 18 | 15 |
| L75 | 31 | 24 |
| BUSCO (%) | 99.2 | 99.1 |
| Single | 2.6 | 2.5 |
| Duplicated | 96.6 | 96.6 |
| Fragmented | 0 | 0.1 |
| Missing | 0.8 | 0.8 |
| Base accuracy (QV by Merqury) | 69.9 | 68.5 |
| Final assembly | | |
| Assembled genome size (Mb) | 784.9 | 781.0 |
| Number of anchored contigs | 137 | 86 |
| BUSCO (%) | 99.1 | 99.1 |
| Single | 2.5 | 2.3 |
| Duplicated | 96.6 | 96.8 |
| Fragmented | 0 | 0.1 |
| Missing | 0.9 | 0.8 |
| Number of high-confidence genes | 104,350 | 103,942 |

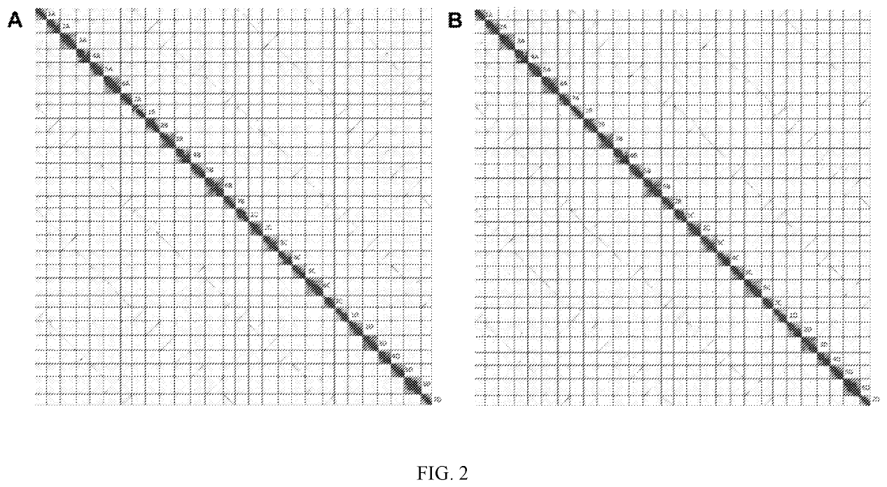

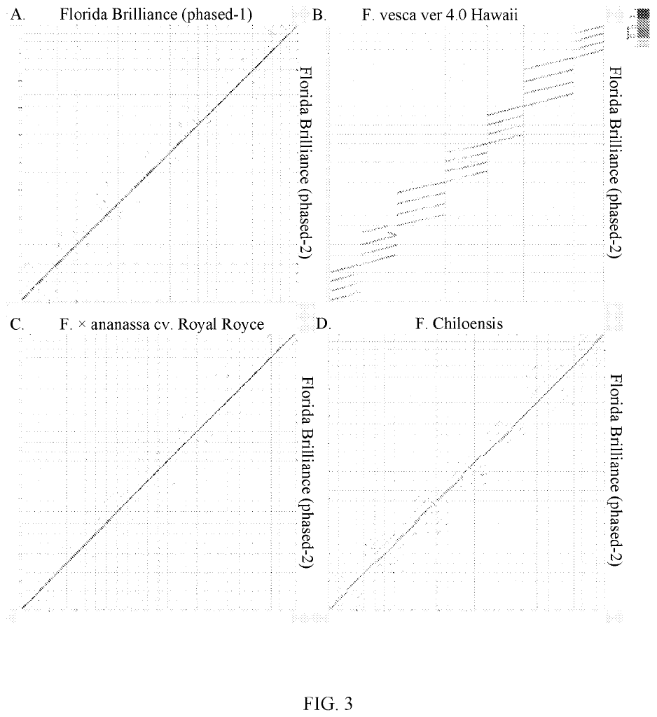

Hi-C reads were re-analyzed to scaffold the phased-1 and phased-2 assembly. Among 286 M of Hi-C read pairs, 92.6% of Hi-C R1 and 91.7% of Hi-C R2 reads were mapped to assembled genome (Table 4). Unmapped read pairs, and low quality of pairs, pairs with singleton were excluded, 44.7% of uniquely mapped read pairs were used for scaffolding using SALSA2. The unique Hi-C read pairs were visualized on Hi-C contact map, possessing 28 pairs of chromosomes on phase-1 and phased-2 genome assemblies (FIG. 2). Intense Hi-C read pairs were evenly distributed against whole genome assembly, indicating a low probability of misassemble sequencing data. Sequence identity between ‘Florida Brilliance’ and published reference genomes were visualized (FIG. 3). At first, we confirmed collinearity between phased-1 and phased-2 genome assemblies of ‘Florida Brilliance’ (FIG. 3A). Alignments of phased-2 assembly against the diploid F. vesca v4.0 showed a high degree of collinearity with the exception of major translocations on IA and 2C (FIG. 3B). In particular, ‘Florida Brilliance’ sub-genome A showed higher sequence similarity with F. vesca than other sub-genomes, which agrees with the finding from Hardigan et al (2022). Alignments of ‘Florida Brilliance’ phased-2 assembly against the FaRR1 also displayed a high degree of collinearity, suggesting that the two high-quality reference genomes could be effectively used and valuable resource for octoploid strawberry research and breeding (FIG. 3C). The assembly of FaFB2 genome showed translocations on 3C, 4A, 4B and 4C comparing with the genome of F. chiloensis (FIG. 3D). After confirming the sequence identity with FaRR1, we followed the chromosome nomenclature proposed reflecting the proposed diploid origins of each respective sub-genome (A. B, C. D) (Hardigan M A et al. Unraveling the complex hybrid ancestry and domestication history of cultivated strawberry. Molecular biology and evolution 38, 2285-2305 (2021)) based on the sequence similarity with FaRR1 genome. From each of the 56 pseudo-chromosomes in combined phased-1 and phased-2 genome assembly, we selected the most continuous pseudomolecule to produce a phased genome assembly and labelled them as FaFB2. The genetic position and physical coordinates of markers were collinear demonstrating good agreement between FaFB2 assembly and ‘Florida Brilliance’ genetic linkage map (FIG. 4A).

TABLE 4
Statistics of genome assembly and Hi-C analysis
for octoploid strawberry ‘Florida Brilliance’.

| Category | Statistics | Proportion (%) |
|---|---|---|
| Number of R1 reads mapped | 265,085,428 | 92.6 |
| Number of R2 reads mapped | 262,663,135 | 91.7 |
| Number of uniquely mapped pairs | 128,100,643 | 44.7 |
| Number of unmapped read pairs | 8,661,036 | 3.0 |
| Number of low quality of pairs | 121,926,069 | 42.6 |
| Number of pairs with singleton | 27,695,139 | 9.7 |
| Total number of pairs processed | 286,382,887 | 100.0 |

Telomeric motif (5′-TTTAGGG-3′) enriched in the terminal parts of the pseudo-chromosomes allowed the identification of 103 telomeres (FIG. 5). Putative telomeric sequences were found at the 5′ and/or 3′ ends in all 56 pseudo-chromosomes, and 50 were assembled telomere-to-telomere (FIG. 5). Two chromosome pseudomolecules are potentially telomere-to-telomere, but the putative telomeric sequences are located 6 Mb or 7 Mb from end of chromosome 7A. Seven chromosomes had a short interstitial telomere-like sequence (FIG. 5).The gene distribution along the chromosome followed the typical distribution of monocentric plant genomes and the positions of centromere were almost similar between phased-1 and phased-2 assembly (FIG. 5).

To evaluate both phased-1 and phased-2 genome assembly based on assembly of core eudicots gene, the genome quality was assessed using LTR assembly Index (LAI) (Ou S et al. Assessing genome assembly quality using the LTR Assembly Index (LAI). Nucleic acids research 46, e126-e126 (2018)). LAI for each chromosome was ranged from 17.17 to 20.13. LAI for combined phased-1 and phased-2 genome was 19.72 (Table 5). Synteny analysis between ‘Florida Brilliance’ and F. vesca showed conserved synteny with the exception of major translocations on 1B, ID, and 2C as obtained by whole genome alignment (FIG. 6).

TABLE 5
LTR Assembly Index (LAI) scores for ‘Florida Brilliance’
phase-1 and phase-2 subgenomes.

| Genome assembly | Sub-genome | Haplotype phase | LAI |
|---|---|---|---|
| FloridaBrilliance | A | Haplotype-1 | 19.72 |
| FloridaBrilliance | A | Haplotype-2 | 19.27 |
| FloridaBrilliance | B | Haplotype-1 | 17.17 |
| FloridaBrilliance | B | Haplotype-2 | 18.44 |
| FloridaBrilliance | C | Haplotype-1 | 18.83 |
| FloridaBrilliance | C | Haplotype-2 | 19.17 |
| FloridaBrilliance | D | Haplotype-1 | 19.75 |
| FloridaBrilliance | D | Haplotype-2 | 20.13 |

| | |
|---|---|
| Total | 19.72 |

Our smudgeplot displays genome structure of cultivated strawberry based on K-mer analysis supports genomic structure of ‘AAAAAABB’, similar with ‘AvAvBiBiBIB1B2B2’. When examined by sub-genome, we observed sub-genome dominance: A>B>C>D based on the proportion of complete vs fragmented or missing genes in ‘Florida Brilliance’ genome assembly, as with ‘Royal Royce’ (Hardigan M A et al. 2021), and F. chiloensis (Cauret C et al. Chromosome-scale assembly with a phased sex-determining region resolves features of early Z and W chromosome differentiation in a wild octoploid strawberry. G3 Genes| Genomesl Genetics (2022)). Genome structure ‘AAAAAABB’ along with sub-genome dominance might support the model hypothesizing four sub-genomes of octoploid originates from diploid cytoplasm donor F. vesca. one with diploid F. iinunae, and two with unknown diploid close to F. iinumae.

Since the first report of chromosome-scale reference genome by Edger (Edger P P et al. 2019), several octoploid strawberry (F.×ananassa) reference genomes using HiFi reads have been reported and available (Edger P P et al. 2019: Hardigan M A et al. 2021; Lee H-E et al. Chromosome level assembly of homozygous inbred line ‘Wongvo 3115’ facilitates the construction of a high-density linkage map and identification of QTLs associated with fruit firmness in octoploid strawberry (Fragariax ananassa). Frontiers in plant science 12, 696229 (2021); and Shirasawa K et al. A chromosome-scale strawberry genome assembly of a Japanese variety, Reikou. bioRxiv (2021)). The high accuracy of HiFi reads simplified and improved the initial assembly quality for octoploid strawberry with a heterozygous genome. Notably, N50 of ‘Florida Brilliance’ (23.7 and 26.7 Mb) in phased-1 and phased-2 initial assembly, were much higher than those of genome assembly studies reported; Wongyo3115 (9.84 Mb). ‘Royal Royce’ (11 Mb), F. chiloensis (10.8 Mb), and ‘Reikou’ (3.9 Mb). In addition, 15 contigs accounted for 50% of the phased-2 genome assembly, indicating a high degree of continuity in the initial assembly. Notably, multiple contigs with being lengths similar to scaffold length after scaffolding indicate multiple single-contig chromosome assemblies (FIG. 7). The final assembly of ‘Florida Brilliance’ consisted of 784.9 Mb and 781.0 Mb in phased-1 and phased-2 assembly, which is in accordance with assembly length of ‘Royal Royce’ (784 Mb) (Hardigan M A et al. 2021). ‘Florida Brilliance’ genome size is in accordant with estimated genome size of 788-804 Mb (Lee H-E et al. 2021). When comparing the corresponding chromosome lengths between ‘Florida Brilliance’, ‘Royal Royce’, and ‘Camarosa’, we confirmed a high similarity between Royal Royce and ‘Florida Brilliance’ (FIG. 8). The results including good contig continuity and similar phased assembly size demonstrate that high-quality haplotype-phased genome assemblies of cultivated strawberries with heterozygous and ploidy can be successfully generated without parental data (Cheng H et al. 2021).

Telomeres, basic structure of eukaryotic chromosomes, are typically tandemly arranged mini-satellites following the formula (TxAyGz)n at both ends of chromosomes (Peska V et al. Origin, diversity, and evolution of telomere sequences in plants. Frontiers in Plant Science 11. 117 (2020)). Telomere-to-telomere genome assemblies are necessary to ensure that all variants are discovered. Recently. although putative telomeric sequences (5′-TTTAGGG-3′) of octoploid strawberry were first found at both ends in 7 of the 28 pseudo-chromosomes of F. chiloensis (Cauret C et al. 2022). Overall, 90% of putative telomeres were found in combined phased-1 and phased2 genome assemblies for ‘Florida Brilliance’ (FIG. 1) and were indicative of a high-quality assembly.

LAI scores for combined phased-1 and phased-2 assemblies were 19.72, comparable to ‘Royal Royce’ and F. vesca Hawaii 4 (Hardigan M A et al. 2021), given that LAI score greater than 20 is considered quality of ‘gold standard’. Genome assembly based on NGS technologies typically possess very low sequence continuity compared to Sanger-based technique or BAC-based scaffoldings (Ou S et al. 2018). BUSCO score of ‘Florida Brilliance’ (99.2%) is highest among published reference octoploid strawberry genomes such as ‘Camarosa’ (96.2%), ‘Royal Royce’ (98.1%), ‘Wongyo 3115’ (94.1%), F. chiloensis (99.1%), indicating a high level of completeness. Both LAI and BUSCO scores indicate high-quality assembly of ‘Florida Brilliance’. In the combined phased-1 and phased-2 genome assembly, ‘Florida Brilliance’ genome consisted of 652 Mb of repetitive sequence accounting for 41.66%, which is higher than published reference genome such as ‘Royal Royce’ (38.4%), ‘Camarosa’ (36%), ‘Wongyo 3115’ (38.75%).

Several chromosomal rearrangements in the F. chiloensis reference relative to the F. vesca and ‘Royal Royce’ (F.×ananassa) have been reported (Cauret C et al. 2022) and are apparently visible when compared to our assembly (FIG. 3). For example, the alignment against F. chiloensis shows disagreement in continuity in 3 chromosomes (Fchil3-B2, Fchil4-Av, and Fchil4-Bi) (FIG. 3D). The chromosomal rearrangements were not found in 3 chromosomes between ‘Florida Brilliance‘ and’Royal Royce’. However, further investigation should be necessary to resolve whether these disagreements are misassembles or true rearrangement.

### Example 4. Transcriptome Assembly and Gene Annotation

In total, 41.66% of the combined phased-1 and phased-2 assemblies were annotated as repetitive sequence. The majority of this repeat sequence was contributed by LTR class transposable elements (Table 6). Among them. 223,060 simple sequence repeats (SSRs) representing 0.16% of whole genome sequence were found (Table 7). We obtained a redundant set of 321,419 transcripts by aligning Iso-Seq and RNA-Seq datasets to the combined phased-1 and phased-2 assemblies. Among them, a non-redundant set of 171,404 transcripts was retained after filtering duplicated transcripts. BUSCO analysis of the transcript assemblies revealed uncovering of 2,209 complete core eudicots genes (94.9%. 0.9% single-copy. 94% duplicated) with 0.8% fragmented and 4.3% missing core eudicots genes (Table 8). Custom repeat libraries and non-redundant set of transcripts were provided for gene annotation on GenSAS v6.0 (Humann J L et al. 2019). Integrating ab initio predictions and evidence-based (RNA-Seq and Iso-Seq) prediction, 208,292 genes remained in the final annotation with 104,350 models in the phase-1 assembly, 103,942 models in the phase-2 assembly, and 119,076 in the FaFB2 assembly. When classifying all predicted genes by sub-genome, sub-genome A occupied 27.4% of the entire genome (Tables 9 and 10).

TABLE 6
Classification and distribution of repetitive DNA elements identified in
the genome assembly for ‘Florida Brilliance’ by EDTA pipeline.

| | | | | Length | Proportion |
|---|---|---|---|---|---|
| | | | | of masked | of masked |
| | | | | sequence | sequence |
| | Class | Sub-Class | Count | (bp) | (%) |
| LTR | Class I | | | | |
| | | Copia | 99,547 | 73,506,892 | 4.69 |
| | | Gypsy | 174,983 | 184,244,965 | 11.77 |
| | | unknown | 286,120 | 149,607,484 | 9.55 |
| TIR | Class II | | | | |
| | | CACTA | 148,100 | 59,217,274 | 3.78 |
| | | Mutator | 215,008 | 67,488,697 | 4.31 |
| | | PIF_Harbinger | 55,008 | 20,728,437 | 1.32 |
| | | Tc1_Mariner | 4,971 | 1,709,712 | 0.11 |
| | | hAT | 84,543 | 33,197,559 | 2.12 |
| NonTIR | Class II | | | | |
| | | helitron | 142,472 | 62,596,795 | 4.00 |
| Total | — | — | 1,210,752 | 652,297,815 | 41.66 |

TABLE 7
Simple sequence repeats of octoploid strawberry genome assembly
for ‘Florida Brilliance’ using SSR Finder.

| | | | | Genome |
|---|---|---|---|---|
| | Frequency | Proportion | Size | coverage |
| SSR type | (#) | (%) | (bp) | (%) |
| Dinucleotide | 80,951 | 36.29 | 820,359 | 0.05 |
| Trinucleotide | 52,060 | 23.34 | 593,417 | 0.04 |
| Tetranucleotide | 65,389 | 29.31 | 737,447 | 0.05 |
| Pentanucleotide | 15,126 | 6.78 | 217,899 | 0.01 |
| Hexanucleotide | 9,533 | 4.27 | 164,112 | 0.01 |
| Total | 223,060 | 100.00 | 2,533,234 | 0.16 |

TABLE 8
Benchmarking universal single-copy orthologs (BUSCO) analysis
of 171,404 transcriptome assemblies for ‘Florida Brilliance’.

| | embryophyta_odb10 | eudicots_odb10 |
|---|---|---|
| Complete BUSCOs (C) | 1,540 (95.4%) | 2,209 (94.9%) |
| Complete and single-copy BUSCOs (S) | 16 (1.0%) | 22 (0.9%) |
| Complete and duplicated BUSCOs (D) | 1,524 (94.4%) | 2,187 (94%) |
| Fragmented BUSCOs (F) | 13 (0.8%) | 19 (0.8%) |
| Missing BUSCOs (M) | 61 (3.8%) | 98 (4.3%) |
| Total BUSCO groups searched | 1,614 (100.0%) | 2,326 (100.0%) |

TABLE 9
Genes predicted in the combined phased-1 and phased-2 genome assembly of ‘Florida Brilliance’.

| | Sub-genome in phased-1 assembly | | | | | Sub-genome in phased-2 assembly | | | | |
|---|---|---|---|---|---|---|---|---|---|---|
| Chromosome | A | B | C | D | Total | A | B | C | D | Total |
| Chr01 | 3,327 | 3,042 | 3,084 | 2,951 | 12,404 | 3,264 | 3,069 | 2,875 | 3,169 | 12,377 |
| Chr02 | 4,327 | 3,898 | 3,658 | 3,860 | 15,743 | 4,237 | 3,868 | 3,657 | 3,795 | 15,557 |
| Chr03 | 4,613 | 3,854 | 3,857 | 3,716 | 16,040 | 4,491 | 3,943 | 3,859 | 3,757 | 16,050 |
| Chr04 | 3,718 | 3,373 | 3,127 | 2,988 | 13,206 | 3,651 | 3,380 | 3,220 | 3,036 | 13,287 |
| Chr05 | 3,881 | 3,712 | 3,262 | 3,324 | 14,179 | 3,872 | 3,737 | 3,312 | 3,252 | 14,173 |
| Chr06 | 5,321 | 4,925 | 4,675 | 4,414 | 19,335 | 5,325 | 4,876 | 4,681 | 4,426 | 19,308 |
| Chr07 | 3,471 | 3,294 | 3,661 | 3,017 | 13,443 | 3,464 | 3,402 | 3,302 | 3,022 | 13,190 |
| Total | 28,658 | 26,098 | 25,324 | 24,270 | 104,350 | 28,304 | 26,275 | 24,906 | 24,457 | 103,942 |
| Percentage (%) | 27.5 | 25 | 24.3 | 23.3 | 100 | 27.2 | 25.3 | 24 | 23.5 | 100 |

TABLE 10
Genes predicted in the FaFB2 of ‘Florida Brilliance’.

| | Genes predicted in the FaFB2 of ‘Florida Brilliance’. | | | | |
|---|---|---|---|---|---|
| Chromosome | A | B | C | D | Total |
| Chr01 | 3,327 | 3,042 | 3,084 | 2,951 | 12,404 |
| Chr02 | 4,327 | 3,898 | 3,658 | 3,860 | 15,743 |
| Chr03 | 4,613 | 3,854 | 3,857 | 3,716 | 16,040 |
| Chr04 | 3,718 | 3,373 | 3,127 | 2,988 | 13,206 |
| Chr05 | 3,881 | 3,712 | 3,262 | 3,324 | 14,179 |
| Chr06 | 5,321 | 4,925 | 4,675 | 4,414 | 19,335 |
| Chr07 | 3,471 | 3,294 | 3,661 | 3,017 | 13,443 |
| Total | 28,658 | 26,098 | 25,324 | 24,270 | 104,350 |
| Percentage (%) | 27.5 | 25 | 24.3 | 23.3 | 100 |

The total gene number of FaFB2 was 104.099 (Table 10). similar to that of ‘Royal Royce’ (101,721) and ‘Camarosa’ (108,087), which is very different from that of -Wongyo 3115′ (151,892) and ‘Reikou’ (167,721). The different number of genes among published genomes could have resulted from the difference in the implementation of default parameters for the gene prediction. Although the total number of genes differs among the genomes available. Sub-genome A representing diploid progenitor F. vesca occupied the largest proportion (27.5%) (Table 8), similar to ‘Royal Royce’ (270%) (Hardigan M A et al. 2021). We also confirmed the sub-genome dominance (A>B>C>D) based on the total number of genes estimated for each sub-genome (Tables 9 and 10).

### Example 5. BAC Library Screening for Clone Identification Associated with FaCal

Primers flanking each SNP that aligned to all or most of Fvb 6 subgenomes and showed slight background and strong brightness when visualized in 1% agarose gels were chosen to screen the BAC libraries. One pair of primers was chosen for SNP AX-89896208, AX-89808208 and AX-89838962, and three pair of primers were chosen for the 9-InDel close to SNP AX-89838986 (Table 11). The positive PCR reaction was done using primer AM001-C12-M13 (Table 11). The positive PCR control primers were used according to the provider's recommendation. After Round I PCR of the six BAC libraries. 23. 34 and 26 positive hits in BamHI-treated libraries from 11.77-96, ‘Florida Brilliance’ and 14.101-225, respectively, showed PCR amplification. Likewise, 18, 15 and 27 positive hits were chosen from HindIII-treated libraries from 11.77-96, ‘Florida Brilliance’ and 14.101-225, respectively. In total, 143 PCR products were observed in 1% agarose gels.

TABLE 11
Primers for BAC library screening designed close to three SNPs and one 9-bp
InDel in FaRCal in Fvb6-3 and homologous regions in subgenomes Fvb6-1,
Fvb6-2, and Fvb6-4 of ‘Camarosa’.

| | | | | |
|---|---|---|---|---|
| | | SEQ | | SEQ |
| | | ID | | ID |
| Name | Forward Primer (5′-3′) | NO: | Reverse Primer (5′-3′) | NO: |

SNP1 AX-89896208

| | | | | |
|---|---|---|---|---|
| SNP1_P1* | GCACCCTCTGATGTGATTCTAT | 5 | GTTGTGATGGCTTGTAGGAGTA | 8 |
| SNP1_P2 | TTTGATGGGCCAGTCATCTC | 6 | GTTGCTCTTGCCAAGGTTTC | 9 |
| SNP1_P3 | CCAAGAGGAATACAGCAAGG | 7 | CAGGAGCAAGAGAATGAGTATG | 10 |

Indel close to AX-89838986

| | | | | |
|---|---|---|---|---|
| SNP2_P1* | CAAGCTCCACATCATCCTATT | 11 | GGTGGTAGGGCAATAATACTC | 14 |
| SNP2_P2 | CCATCGAATGTCTTCCTCTTC | 12 | CGGGTTTATTCACTGCTTACT | 15 |
| SNP2_P3 | CTACCACCTAAGCTTTCCATAC | 13 | GTACTCGAGGACAATGACATAC | 16 |

SNP3 AX-89808208

| | | | | |
|---|---|---|---|---|
| SNP3_P1* | CTTCTGGCTGATGTGTTGT | 17 | TCCTCCAATTGCTCGTTTAG | 20 |
| SNP3_P2 | TGCTCGTACGTTACTTTACTG | 18 | CCTGAATTCCTGGCCATATC | 21 |
| SNP3_P3 | GGTCTCAAGAGAGAGACGTAAA | 19 | CCTGGCCATATCAAACCAATAG | 22 |

SNP4 AX-89838962

| | | | | |
|---|---|---|---|---|
| SNP4_P1* | TCTGCACCACCACATTTC | 23 | GTAGGCCATTGGGAATAAG | 26 |
| SNP4_P2 | GTGGCCTCATTACCCTAATAAC | 24 | CTCCTTCCCAAACCCATTAA | 27 |
| SNP4_P3 | AAGAATCTCTCCCACTCTCTC | 25 | GGTAATGAGGCCACACATAAA | 28 |

Positive Control

| | | | | |
|---|---|---|---|---|
| AM001-C12- | ATATAATGCAAAAAGTGGCT | 29 | GTAATGACCCTTTTCTCTCC | 30 |
| M13* | | | | |

*Primers with best performance
Positive control primer was used according to Amplicon Express.

After Round II PCR of the six BAC libraries, 237 positive hits showed HRM amplification from which only 139 were unique BAC clones. Thirty-six, 39 and 16 correspond to BamHI-treated libraries 11.77-96, ‘Florida Brilliance’ and 14.101-225, respectively. Fifteen, 20 and 13 clones were obtained from HindIII-treated libraries 11.77-96, ‘Florida Brilliance’ and 14.101-225, respectively.

The 139 BAC clones were re-screened with a subgenome-specific HRM marker for an InDel previously designed for MAS to differentiate BAC clones containing resistant and susceptible alleles of FaRCa1 in Fvb 6-3. Plasmid DNA from four BAC clones that showed HRM curves corresponding to the resistant allele and from four BAC clones with HRM curves corresponding to the susceptible allele were sent to Sanger end-sequencing using T7 and M13 reverse primers (GENEWIZ, South Plainfield, NJ, USA). Sequences of plasmid DNA were blasted to subgenomes Fvb 6-1, 6-2, 6-3 and 6-4. Four BAC clones showed subgenome specificity to Fvb6-3. Two BAC clone curves (67 or SL_CO001 and 75 or SL_C002) presented resistant allele HRM and two BAC clones (110 or SL_C003 and 111 or SL_C004) showed susceptible allele HRM curves. BAC clones SL_C001 and SL_C002 derived from the ‘Florida Brilliance’ BamHI-treated library. BAC clone SL_C003 and SL_CO004 derived from the ‘Florida Brilliance’ HindIII-treated library and the 14.101-225 BamHI-treated library, respectively.

DNA libraries were constructed for plasmid DNA from BAC clones SL_C001, SL_C002, SL_C003 and SL_C004. Pair-end sequencing using Illumina high-throughput sequencer produced 1.3, 1.5, 1.4 and 1.7 clean base (G) data for SL_CO001, SL_C002, SL_C003 and SL C004, respectively (Table 12). Sequencing data quality obtained is summarized in Table 12.

TABLE 12
Data quality summary.

| | | | Raw | Clean | Effective | Error | | | GC |
|---|---|---|---|---|---|---|---|---|---|
| | | Clean | Base | Base | Rate | Rate | Q20 | Q30 | Content |
| Sample | Raw Reads | Reads | (G) | (G) | (%) | (%) | (%) | (%) | (%) |
| SL_C001 | 4210677 | 4195716 | 1.3 | 1.3 | 99.64 | 0.03 | 96.68 | 91.28 | 42.07 |
| SL_C002 | 5171894 | 5161136 | 1.6 | 1.5 | 99.79 | 0.03 | 97.35 | 92.52 | 42.36 |
| SL_C003 | 4722045 | 4709806 | 1.4 | 1.4 | 99.74 | 0.03 | 96.94 | 91.77 | 42.62 |
| SL_C004 | 5761597 | 5750586 | 1.7 | 1.7 | 99.81 | 0.03 | 97.22 | 92.31 | 42.9 |

Raw reads: four rows are taken as a unit to calculate the total amount of read1 and read2 in raw data files
Clean reads: four rows are taken as a unit to calculate the total amount of read1 and read2 in clean data files
Raw bases: (total raw reads) * (sequence length), calculating in G
Clean bases: (total clean reads) * (sequence length), calculating in G
Error rate: base error rate
Q20, Q30: (Base count of Phred value > 20 or 30) / (Total base count)
GC content: (G & C base count) / (Total base count)

### Example 6. Haplotype Alignment and Candidate Gene Detection

Four Illumina de novo BAC assembles all surrounding and including the 9-bp InDel marker were constructed independently. Illumina-sequenced reads from susceptible SL_003 and SL_004 samples de novo assembled into a major contig of about 99.7 kb and 104 kb in length each. These major contigs matched the subgenomic region of FaRCa1 in ‘Camarosa’ reference genome v1.0 and to contig 19 of size 35.9 Mb from the ‘Florida Brilliance’ HiFi assembly. Illumina sequenced reads from resistant SL_001 and SL_002 samples de novo assembled into six and four major contigs, respectively. Contig sizes ranged between 1.6 kb to 61 kb, and 5.5 kb to 55 kb in SL 001 and SL 002, respectively. To link these disconnected assemblies corresponding to the resistant BAC clones, three ‘Florida Brilliance’ PacBio contigs that span most of the FaRCa1 region with sizes of 50 kb, 70 kb, 80 kb and contig 14380 of size 6.74 Mb from the ‘Florida Brilliance’ HiFi assembly were utilized as references.

Resistant haplotypes aligned to 183 kb of the ‘Camarosa’ FaRCa1 region and susceptible haplotype assemblies aligned to 117 kb of the FaRCa1 region (FIG. 4A). ‘Florida Brilliance’ PacBio contigs and ‘Florida Brilliance’ HiFi assembly were also used to confirm a number of large and small indels in the resistant and susceptible haplotypes.

‘Camarosa’ annotated genomes 3,143 were used to locate genes in the FaRCa1 region using JBrowse 1.16.4 from the GDR web page147. Sequences of annotated genes located in the region were used as BLAST query in NCBI data sets. Twenty-one genes were found in the region which show diverse predicted biological functions (FIG. 4A and Table 13). Three annotated genes had potential roles in disease resistance. The first gene named as augustus masked-Fvb6-3-processed-gene-163.0 according to the ‘Camarosa’ genome annotation v1.03, or FxaC_22g29230 according to the ‘Camarosa’ genome annotation v1.a2143, or gene 163.0 as previously referred in this study, showed high homology with predicted Prunus persica rust resistance kinase Lr10-like (Table 13). The second gene named as maker-Fvb6-3-augustus-gene-163.26 according to the ‘Camarosa’ genome annotation v1.03. or FxaC_22g29220 according to the ‘Camarosa’ genome annotation v1.a2143 and previously referred as gene 163.26 in this study had homology with predicted Rosa chinensis rust resistance kinase Lr10 (Table 13). The third gene named as maker-Fvb6-3-augustus-gene-163.23 according to the ‘Camarosa’ genome annotation v1.03, or FxaC 22g29260 according to the ‘Camarosa’ genome annotation v1.a2143 or simply gene 163.23 had homology with a predicted Fragaria vesca subsp. vesca putative leucine-rich repeat receptor-like serine/threonine-protein kinase (Table 13). The two annotated rust resistance kinase and the putative leucine-rich repeat receptor-like serine/threonine-protein kinase gene are located 20 kb and 44.6 kb from the 9-bp InDel selection marker (FIG. 4A).

TABLE 13
Genes located in the 166 kb FaRCa1 region in Fvb 6-3 from the ‘Camarosa’ genome annotation v1.0
and ‘Camarosa’ genome annotation v1.a2. Gene description corresponds to the first alignment
result from BLAST in NCBI webpage. Genes with different positions in both annotations are shown with an *.

| No | Gene Name | Minimum | Maximum | Length | Direction | Gene description | Sequence |
|---|---|---|---|---|---|---|---|
| 1 | FxaC_22g29101 | 16237661 | 16240674 | 3014 | F | PREDICTED: Fragaria vesca subsp. vesca | XM_004303187.2 |
| | | | | | | protein FAR1-RELATED SEQUENCE 3-like | |
| | | | | | | (LOC101299598), transcript variant X2, mRNA | |
| 2 | maker-Fvb6-3-augustus- | 16242589 | 16244183 | 1595 | F | PREDICTED: Fragaria vesca subsp. vesca | |
| | gene-162.37 | | | | | uncharacterized LOC101299312 | |
| | FxaC_22g29160 | 16242589 | 16244183 | 1595 | F | (LOC101299312), mRNA | |
| 3 | maker-Fvb6-3-augustus- | 16243799 | 16248424 | 4626 | R | PREDICTED: Fragaria vesca subsp. vesca | |
| | gene-162.41* | | | | | magnesium transporter MRS2-1 | |
| | FxaC_22g29170 * | 16244812 | 16248583 | 3772 | R | (LOC101299016), mRNA | |
| 4 | maker-Fvb6-3-augustus- | 16250615 | 16253518 | 2904 | R | PREDICTED: Fragaria vesca subsp. vesca | |
| | gene-162.42 * | | | | | chaperone protein dnaJ 8, chloroplastic | |
| | FxaC_22g29180* | 16251485 | 16253583 | 2099 | R | (LOC101298724), transcript variant X2, mRNA | |
| 5 | maker-Fvb6-3-snap-gene- | 16257541 | 16264866 | 7326 | | PREDICTED: Fragaria vesca subsp. vesca | XM_011468714.1 |
| | 162.52 * | | | | | probable protein phosphatase 2C 60 | |
| | FxaC_22g29190 * | 16257528 | 16264855 | 7328 | R | (LOC101311116), transcript variant X4, mRNA | |
| 6 | maker-Fvb6-3-snap-gene- | 16269517 | 16273899 | 4383 | R | PREDICTED: Fragaria vesca subsp. vesca | XM_011470211.1 |
| | 162.54 | | | | | U-box domain-containing protein 51-like | |
| | FxaC_22g29200 | 16269517 | 16273899 | 4383 | R | (LOC101310823), mRNA | |
| 7 | maker-Fvb6-3-augustus- | 16293453 | 16296148 | 2696 | F | PREDICTED: Rosa chinensis rust resistance | XM_024304943.2 |
| | gene-163.26 | | | | | kinase Lr10 (LOC112167853), mRNA | |
| | FxaC_22g29220 | 16293453 | 16296148 | 2696 | R | | |
| 8 | augustus_masked-Fvb6-3- | 16296210 | 16298857 | 2648 | F | PREDICTED: Prunus persica rust resistance | XM_020565685.1 |
| | processed-gene-163.0 * | | | | | kinase Lr10-like (LOC109949610), mRNA | |
| | FxaC_22g29230 * | 16296373 | 16298845 | 2473 | F | | |
| 9 | maker-Fvb6-3-snap-gene- | 16305756 | 16306643 | 888 | F | not found | |
| | 163.31 | | | | | | |
| | FxaC_22g29250 | 16305756 | 16306643 | 888 | F | | |
| 10 | maker-Fvb6-3-augustus- | 16318411 | 16321581 | 3171 | F | PREDICTED: Fragaria vesca subsp. vesca | XM_011468707.1 |
| | gene-163.23 | | | | | putative leucine-rich repeat receptor-like | |
| | FxaC_22g29260 | 16318411 | 16321581 | 3171 | F | serine/threonine-protein kinase At2g24130 | |
| | | | | | | (LOC101309373), mRNA | |
| 11 | maker-Fvb6-3-augustus- | 16335004 | 16336197 | 1194 | F | PREDICTED: Fragaria vesca subsp. vesca | XR_906961.1 |
| | gene-163.24 | | | | | uncharacterized LOC101298143 | |
| | FxaC_22g29270 | 16335020 | 16336206 | 1187 | F | (LOC101298143), ncRNA | |
| 12 | maker-Fvb6-3-augustus- | 16337780 | 16338676 | 897 | R | PREDICTED: Fragaria vesca subsp. vesca | XR_906962.1 |
| | gene-163.25 | | | | | uncharacterized LOC101309659 | |
| | FxaC_22g29280 | 16337780 | 16338676 | 897 | F | (LOC101309659), ncRNA | |
| 13 | maker-Fvb6-3-snap-gene- | 16338944 | 16340861 | 1918 | | PREDICTED: Fragaria vesca subsp. vesca | XM_011468710.1 |
| | 163.37 * | | | | | shikimate O-hydroxycinnamoyltransferase | |
| | FxaC_22g29290 * | 16339170 | 16340861 | 1692 | R | (LOC101298438), mRNA | |
| 14 | snap_masked-Fvb6-3- | 16354928 | 16358047 | 3120 | R | PREDICTED: Fragaria vesca subsp. vesca | XM_011468710.1 |
| | processed-gene-163.18 | | | | | shikimate O-hydroxycinnamoyltransferase | |
| | FxaC_22g29350 | 16354928 | 16358047 | 3120 | R | (LOC101298438), mRNA | |
| 15 | maker-Fvb6-3-snap-gene- | 16370948 | 16375895 | 4948 | R | PREDICTED: Fragaria vesca subsp. vesca | XM_004303179.2 |
| | 163.40 * | | | | | organic cation/carnitine transporter 4 | |
| | FxaC_22g29360 * | 16370854 | 16376165 | 5312 | R | (LOC101296978), mRNA | |
| 16 | maker-Fvb6-3-augustus- | 16383479 | 16390041 | 6563 | F | PREDICTED: Rosa chinensis protein HEADING | XM_024329286.2 |
| | gene-164.31 * | | | | | DATE REPRESSOR 1 (LOC112189901), mRNA | |
| | FxaC_22g29380 * | 16383494 | 16386259 | 2766 | F | | |
| 17 | maker-Fvb6-3-augustus- | 16383612 | 16390384 | 6773 | R | PREDICTED: Fragaria vesca subsp. vesca | XM_004303176.2 |
| | gene-164.36 * | | | | | uncharacterized LOC101296120 | |
| | FxaC_22g29390 * | 16386284 | 16390885 | 4602 | R | (LOC101296120), mRNA | |
| 18 | snap_masked-Fvb6-3- | 16394187 | 16396431 | 2245 | R | PREDICTED: Rosa chinensis F-box/kelch-repeat | XM_024328278.2 |
| | processed-gene-164.19 | | | | | protein At3g23880 (LOC112189018), mRNA | |
| | FxaC_22g29400 | 16394187 | 16396431 | 2245 | R | | |
| 19 | maker-Fvb6-3-augustus- | 16397561 | 16401856 | 4296 | F | PREDICTED: Fragaria vesca subsp. vesca | XM_004303175.2 |
| | gene-164.32 | | | | | pentatricopeptide repeat-containing protein | |
| | FxaC_22g29410 | 16397561 | 16401856 | 4296 | F | At3g02330 (LOC101295637), mRNA | |
| 20 | maker-Fvb6-3-augustus- | 16402755 | 16406336 | 3582 | F | PREDICTED: Fragaria vesca subsp. vesca | XM_004303174.2 |
| | gene-164.33 * | | | | | protein ASPARTIC PROTEASE IN GUARD | |
| | FxaC_22g29420 * | 16402911 | 16406442 | 3532 | F | CELL 2 (LOC101295348), mRNA | |
| 21 | maker-Fvb6-3-augustus- | 16405557 | 16410157 | 4601 | R | PREDICTED: Fragaria vesca subsp. vesca | XM_011468700.1 |
| | gene-164.37 * | | | | | probable folate-biopterin transporter 3 | |
| | FxaC_22g29430 * | 16405421 | 16410004 | 4584 | R | (LOC101308203), transcript variant X2, mRNA | |

The ‘Camarosa’ annotated genome, susceptible and resistance BAC contigs, ‘Florida Brilliance’ PacBio contigs, ‘Florida Brilliance’ Hifi contigs and ‘Florida Brilliance’ RNAseq data from different parts of the plant were used to confirm gene structure of candidate genes in FaRCa1. Sequence alignments between susceptible and resistant haplotypes of gene 163.26 showed a 26-bp insertion in the first coding region and eight SNPs along the sequence of resistant allele (FIG. 4B). Also, the start codon in the resistant haplotype begins 166-bp later in comparison with the annotation for the ‘Camarosa’ susceptible haplotype and that the second coding DNA sequence in the resistant haplotype is shorter than the corresponding third CDS in the ‘Camarosa’ susceptible haplotype. Alignment of resistant and susceptible haplotypes for gene 163.0 show a 38-bp insertion in the 5′ UTR region and 18 SNPs along the sequence of the resistant genotype (FIG. 4B).

RNAseq data obtained from crown, leaf, root, flower, green fruit, and red fruit from ‘Florida Brilliance’ were aligned to genes 163.0, 163.26, and 163.23. A transcript identical to gene 163.26 was found only in the red fruit RNAseq data. Transcripts for genes 163.0 and 163.23 were not found in the other RNAseq data from other tissues of ‘Florida Brilliance’. These results highlight the spatial and temporal specificity of gene 163.26 in the red receptacle of octoploid strawberry. Results obtained from resistant and susceptible sequences in FaRCa1 and RNA seq data suggested that gene 163.26 appears to have an important role in resistance to C. aculatum.

Resistant allele of gene 163.26 translates to a 670-amino acid protein (FIG. 4C). Susceptible allele translation showed an early stop amino acid in the protein (FIG. 4C). The 670-aa protein belongs to the probable receptor-like serine/threonine-protein kinase LRK10-like protein family (IPR045874) according to the InterPro 87.0 database. This protein is composed by six predicted regions: signal-peptide N-region (1-5 aa), signal peptide H-region (6-17 aa), signal peptide C-region (18-25 aa), non-cytoplasmic domain (26-290 aa), transmembrane region (291-314 aa), and cytoplasmic domain (315-670 aa). The protein kinase domain can be found between acid 352 and 640, highlighted in yellow in FIG. 4C. Along the cytoplasmic domain two sites can be distinguished: the protein kinase ATP binding site (358-380 aa) and the serine/threonine kinase active site (472-484 aa) according to the InterPro 87.0 database results. The main biological function of protein 163.26 is phosphorylation, and the main molecular functions are protein kinase activity and ATP and polysaccharide binding (InterPro 87.0).

### Example 7. Comparative Genomic Analysis of FaRCa1 Subgenomes

Resistant haplotypes SL_001 and SL_002 obtained from ‘Florida Brilliance’ BAC clone alignments were used for comparative genomic analysis with homologous FaRCa1 regions in subgenomes Fvb 6-1, 6-2, 6-3 and 6-4 from ‘Camarosa’ reference genome utilizing the progressiveMauve algorithm in Geneious Prime® 2022.0.1. Resistant alignments, SL000 and SL002. showed ten locally collinear blocks (LCB) when aligned with homologous FaRCa1 regions in Fvb 6-1, 6-2, 6-3 and 6-4 (FIG. 4D). LCBs from SL000 and SL002 had high similarities in structure with chromosome Fvb 6-3 and Fvb 6-1, lower similarity with Fvb6-4 and almost no similarity with chromosome Fvb 6-2 (FIG. 4D). Only small LCBs from the BAC contigs are present in Fvb6-2. Segments of the largest LCBs in Fvb 6-3 and Fvb 6-1 are not homologous with other sequences. There seems to be an inversion of ˜30 kb in the extreme of SL002 and middle part of SL000 when compared with Fvb6-3. Gene 163.26 is in the largest LCB. SL000, SL002, Fvb 6-3 and Fvb 6-1 contain homologous sequences for gene 163.26. The homologues gene in Fvb-1 is maker-Fvb6-1-augustus-gene-166.48 or FxaC_21g33630.

### Example 8. Gene Expression Profiling After C. acutatum Inoculation

Relative expression of gene 163.0 after C. acutatum inoculation was reduced over time in susceptible genotype ca1ca1 and homozygous resistant genotype (Ca1Ca1) 17.20-51 (FIG. 4E). For the heterozygous resistant (Ca1ca1) ‘Florida Brilliance’, the relative expression of gene 163.0 seemed relatively similar at five different times (FIG. 4E). Relative expression of gene 163.26 after C. acutatum inoculation decreased through time in homozygous susceptible (ca1ca1) 16.74-68 (FIG. 4F). Relative expression of gene 163.26 in homozygous resistant Ca1Ca1 and heterozygous resistant Ca1ca1 genotype increased after C. acutatum inoculation and remained at high levels at 24, 48, 72 and 96 hpi (FIG. 4F).

### Example 9. Transient Gene Silencing and Overexpression for Candidate Genes

Differences in resistant and susceptible alleles of genes 163.0 and 163.26 indicated a potential role in resistance. To validate the functional role of these genes in C. acutatum resistance, transient gene expression assay was used. A. tumefaciens containing RNAi constructs targeting each gene, 163.0:RNAi and 163.26:RNAi, was injected in green fruits of homozygous resistant (Ca1Ca1) 17.20-51, heterozygous resistant (Ca1ca1) ‘Florida Brilliance’, and homozygous susceptible (ca1ca1) 16.74-68. In addition, A. tumefaciens containing overexpression constructs targeting 163.26, 163.26: OE. was injected in green fruits of heterozygous resistant (Ca1ca1) and homozygous susceptible (ca1ca1) varieties. A. tumefaciens containing empty vectors was also used as control in each genotype. After Agrobacterium-mediated fruit transformation, green fruit were inoculated with a spore mix of C. acutatum. Internal symptoms of AFR were analyzed in 81-101 fruits per each treatment (FIG. 41). Treatment 163.26: RNAi produced a significantly greater area of internal AFR symptomatic area (cm2) and percentage of internal symptomatic area (%) than 163.0:RNAi and empty vector treatments in genotypes Ca1Ca1 and Ca1ca1 (FIGS. 4J-K). On the contrary, 163.0:RNAi and 163.26:RNAi treatments showed significantly reduced area of internal AFR symptomatic area (cm2) and percentage of internal symptomatic area (%) than the empty vector in the susceptible genotype ca1ca1 (FIGS. 4J-K). Similar trends were observed in external AFR symptomatic area (cm2) in all genotypes (FIGS. 4L-N and FIG. 4O). Furthermore, overexpression of 163.26 reduced disease symptoms significantly after infection of the pathogen (FIG. 4P).

Relative expression of gene 163.0 in silenced fruit was 24% greater, 4% lower, and 35% lower than fruit treated with empty vector in genotypes ca1ca1. Ca1ca1 and Ca1Ca1, respectively (FIG. 4G). Relative expression of gene 163.26 in silenced fruit was 5%, 28% and 22% lower than non-silenced fruit in genotypes ca1ca1, Ca1 ca1 and Ca1 Ca1, respectively (FIG. 4H). Relative expression of gene 163.0 in the silencing experiment (FIG. 4G) appeared lower than relative expression of gene 163.26 (FIG. 4H).

Internal and external symptomatic area increase. along with relative expression decrease of gene 163.26 in silenced fruit treated with 163.26:RNAi in resistant genotypes Ca1ca1 and Ca1Ca1, and results from gene expression profile for gene 163.26 suggest that gene 163.26 is the gene underlying resistance to C. acutatum in strawberry at the FaRCa1 locus.

TABLE 14
List of sequences used for RNAi vector
construction and transformation
verification.

DNA sequence (5′-3′)
FaCa1-163.0-RNAi (RNAi)
ggggACAAGTTTGTACAAAAAAGCAGGCTATGCAGAAAATGCTTC
TGAATTTGTTATGCTGTTGGTTTCTCATAGGCGCAGTGCTAGATG
TAGATATTATTGTGCATGGAGAATCAGTTGGCCCTGAAAATTGCA
CAGAGGTTCGATGTAAACGGCATGGCCCTGTGATCCGATTCCCAT
TTCGACTGAAAGGAATGCAACCACTCCATTGCGGCTACCCGGGCT
TTGATCTGTCATGCACCACGGACAAGCAGACTGTGCTCCAGATCA
ACTCATCATCAGCTGACAAGTTCTTGGTTAAAAGGATTGACTACA
CTGCTCAGGAAATTGAAATATATCTTTATAATAGTCACTATGATT
ATTTCAATGATTTCCAGTATATCTGTATTCCTAGACAGATTTTCG
AGCTCAGTTCCTCTCCATTCGAATAATTTCCTGAGCAGTGTAGTC
AATCCTTTTAACCAAGAACTTGTCAGCTGATGATGAGTTGATCTG
GAGCACAGTCTGCTTGTCCGTGGTGCATGACAGATCAAAGCCCGG
GTAGCCGCAATGGAGTGGTTGCATTCCTTTCAGTCGAAATGGGAA
TCGGATCACAGGGCCATGCCGTTTACATCGAACCTCTGTGCAATT
TTCAGGGCCAACTGATTCTCCATGCACAATAATATCTACATCTAG
CACTGCGCCTATGAGAAACCAACAGCATAACAAATTCAGAAGCAT
TTTCTGCATACCCAGCTTTCTTGTACAAAGTGGTcccc
(SEQ ID NO: 31)
FaCa1-163.26-RNAi (RNAi)
ggggACAAGTTTGTACAAAAAAGCAGGCTATGGCTTGTATAGAGC
CACATAACATAAACAATCTCCCAAACATTTCTATTTACCCTTATC
ATCTCTGTTTCATTACGCCTTCTTCCTGCTCACCATGTCTAGAAG
AAGTCTCCTTTTTGCTTCTTACAGTTATTTCACAATCGTCGGATA
CCGGAAATCCTTTAGATATCTGTACCTCTTCTTGTGGAGGTATTT
ACATAAGCTATCCATTTCGACTGAAAGGAGACCCTCAAAACTGTG
GAAACGAAAAGTTTGAGCTGTCTTGCGAGGCAAATAGTACTACTG
CAGTACTATACTTGGAATCAGAAGAGTATTATGTGAAGTCGATCC
ATTACAATAACTTCACAATCCGAATCGTGGATTCCGGTGTTCAGA
AGAAGCGGAACTACTGCTCCACCGCAAGTATAGTACTGCAGTAGT
ACTATTTGCCTCGCAAGACAGCTCAAACTTTTCGTTTCCACAGTT
TTGAGGGTCTCCTTTCAGTCGAAATGGATAGCTTATGTAAATACC
TCCACAAGAAGAGGTACAGATATCTAAAGGATTTCCGGTATCCGA
CGATTGTGAAATAACTGTAAGAAGCAAAAAGGAGACTTCTTCTAG
ACATGGTGAGCAGGAAGAAGGCGTAATGAAACAGAGATGATAAGG
GTAAATAGAAATGTTTGGGAGATTGTTTATGTTATGTGGCTCTAT
ACAAGCCATACCCAGCTTTCTTGTACAAAGTGGTcccc
(SEQ ID NO: 32)
163.0_F1 (construct validation)
CTTCTGAATTTGTTATGCTGTT (SEQ ID NO: 33)
163.0_Rl (construct validation)
CGTTTACATCGAACCTCTG (SEQ ID NO: 34)
163.26_F2 (construct validation)
GCTTGTATAGAGCCACATAAC (SEQ ID NO: 35)
163.26_R2 (construct validation)
CATGGTGAGCAGGAAGA (SEQ ID NO: 36)

TABLE 15
List of primers sequences used for qRT-PCR.

| | | | SEQ ID |
|---|---|---|---|
| No | Name | Primer Sequences (5′-3′) | NO. |
| 1 | FaGapDH2-F | CCCAAGTAAGGATGCCCC | 37 |
| | | CATGTTCG | |
| | FaGapDH2-R | TTGGCAAGGGGAGCAAGA | 38 |
| | | CAGTTGGTAG | |
| 2 | 163.0-F1 | TTCTCAGACGCCCAAAGAAA | 39 |
| | 163.0-R1 | AGTGGACAGAAGGATGGAGA | 40 |
| 3 | 163.26-F3 | CCCGTTAGCTTGGGACTTAC | 41 |
| | 163.26-R3 | AATTGGGTCAAGGAGGCTATG | 42 |

TABLE 16
163.0 and 163.26 cDNA and amino acid sequences.

163.0 cDNA sequence (SEQ ID NO: 1)
ATGCTTCTGAATTTGTTATGCTGTTGGTTTCTCATAGGCGCAGTGCTAGATGTAGATATTATTGTGCATGGAG
AATCAGTTGGCCCTGAAAATTGCACAGAGGTTCGATGTAAACGGCATGGCCCTGTGATCCGATTCCCATTTCG
ACTGAAAGGAATGCAACCACTCCATTGCGGCTACCCGGGCTTTGATCTGTCATGCACCACGGACAACCAGACT
GTGCTCCAGATCAACTCATCATCAGCTGACAAGTTCTTGGTTAAAAGGATTGACTACACTGCTCAGGAAATTG
AAATATATCTTTATAATAGTCACTATGATTATTTCAATGATTTCCAGTATATCTGTATTCCTAGACAGATTTT
CGAGCTCAGTTCCTCTCCATTCGAATTCTCAGACGCCCAAAGAAAAACTAACTATACCATATTCAGTTGTCCA
CCGTCAGATAAAAGAGATCGGTCTCCATCCTTCTGTCCACTGGCAAAACTGAGCCCTTGCCTTGGCAATCATA
GCAACAGGATGTATGCTCTTCAGAATGGATGTGCAGAGCTTAAATACATGCCCCAACTGGTGTCATGTACCAA
GGTGGAAGATTACATGTCAGTACCATCTGTCTGGATGCAGCCTGTGAAGCTCAAATGGACCAGACCATCGTGT
AAACGTTGCGAAGAGATGGGCAAGAAATGTGGATTTCTGAATCAAGTTGCAGATGATCAGAAAACTGAAGAAA
CTCAATGTTTAGAGGTGCCTAWAGGTCCTAATCGTGGAAWACGAATCTTGGAGATATTAGGCATTTGTGCACT
TGCTACCGTTCTCACAGGAGTGGCTACTGTAATATATTATGTGTATAGTTCTAAGAAGACAGAAGAAGAAAAT
CAACTGAGAATTGAAAAGTTTTTGGATGATTACAGAGCTCTCAAACCCAGTAGATATGCCTATGGTGATATTA
GGCGGATAACAAATCAATTTGAGGACAAGTTGGGTCAGGGAGCTTATGGAACCGTGTACAAAGGAAAGCTTTC
CTCTGAATTACTTGTTGCTGTGAAAATCCTCGACAATTCACACGAGAAGGGGGAAGATTTCATTAATGAGGTG
GGAACAATGGGTCTCATCCACCATGTCAATGTGGTTCGCTTGGTTGGCTTTTGCGCTGATGGATTCATACGAG
CTCTTATATATGAGTATTTACCAAATGGCTCATTGCAGAATTACTTGTCATCTGCAGATGATAAGAACTCATT
TCTTGGTTGGGAAAAGCTGCAAGATATTGCTTTAGGTATAGCTAAAGGAATCGAATATCTTCATCAAGGGTGT
GACCAGAGAATTCTGCATTTTGATATCAAACCTAGTAACATTTTGCTAGACCAAGAGTTCACTCCAAAAGTTT
CTGATTTTGGTCTAGCCAAGTTGTGCTCTAAGGATCAGAGTGCAATATCCATGACTACAGCTAGGGGGACCAT
GGGCTACATTGCACCAGAAGTGTTCTCTAGGAACTTTGGAAACGTATCTTACAAGTCAGATGTATATAGTTTC
GGAATCTTGTTGCTTGAAATGATTGGGGGAAAGAAAAATTTTAAAGTTATGGAGGACTCCACCAGCCATATCT
ACTTCCCAGAATGGATTTATAACCTTTTAGAACAAGGGAGTGATCTACGAATCCATATCGAGGACGAAGAAGA
TGTTATATTAGCTAGAAAACTAGCAATCATCGGTCTATGGTGCATTCAATGGCATCCAATAGATCGCCCATCC
ATGAGCAATGTTGTTCAAATGTTGGAGAGAGAAGGTGACAGTCTAAGCATGCCTCCTTATCCCTTTGCCTCTA
CTTCAAATTGA
163.0 amino acid sequence (SEQ ID NO: 2)
MLLNLLCCWFLIGAVLDVDIIVHGESVGPENCTEVRCKRHGPVIRFPFRLKGMQPLHCGYPGFDLSCTTDNQT
VLQINSSSADKFLVKRIDYTAQEIEIYLYNSHYDYENDFQYICIPRQIFELSSSPFEFSDAQRKTNYTIFSCP
PSDKRDRSPSFCPLAKLSPCLGNHSNRMYALQNGCAELKYMPQLVSCTKVEDYMSVPSVWMQPVKLKWTRPSC
KRCEEMGKKCGFLNQVADDQKTEETQCLEVPKGPNRGKRILEILGICALATVLTGVATVIYYVYSSKKTEEEN
QLRIEKFLDDYRALKPSRYAYGDIRRITNQFEDKLGQGAYGTVYKGKLSSELLVAVKILDNSHEKGEDFINEV
GTMGLIHHVNVVRLVGFCADGFIRALIYEYLPNGSLQNYLSSADDKNSELGWEKLQDIALGIAKGIEYLHQGC
DQRILHFDIKPSNILLDQEFTPKVSDFGLAKLCSKDQSAISMTTARGTMGYIAPEVESRNFGNVSYKSDVYSF
GILLLEMIGGKKNFKVMEDSTSHIYFPEWIYNLLEQGSDLRIHIEDEEDVILARKLAIIGLWCIQWHPIDRPS
MSNVVQMLEREGDSLSM?PYPFASTSN*
163.26 cDNA sequence (SEQ ID NO: 3)
ATGTCTAGAAGAAGTCTCCTTTTTGCTTCTTACACCATCCTCCTCCTCCTTCCTTTTACAGTTATTTCACAAT
CGTCGGATACCGGAAATCCTTTAGATATCTGTACCTCTTCTTGTGGAGGTATTTACATAAGCTATCCATTTCG
ACTGAAAGGAGACCCTCAAAACTGTGGAAACGAAAAGTTTGAGCTGTCTTGCGAGGCAAATAGTACTACTGCA
GTACTATACTTGGAATCAGAAGAGTATTATGTGAAGTCGATCCATTACAATAACTTCACAATCCGAATCGTGG
ATTCCGGTGTTCAGAAGAAGCGGAACTACTGCTCCACCGGACTACACTCTATGACCACCTTCGACTCTAGTGA
TCCATATAGCTTCTCTTCCTTGTACACTCCTTACATGGAACAAGTGCCAATAATACTTGTGACCTGCGCAACT
CCGGTGAATTCCACTCTCTTTATTGAAACAGCTTCATGCCTCAACAGCACCACCATCGATTCTTCCAAATACA
ACTATTTCATGGTTGGCTCTGTAACTTCATTTGATTTGAAGAGTTCTTGTGAAGTAACACAAATGCTTTTAAC
ATCCCCTTCAATCAACAACAGCTTCTTGATGTCATGTGAAAGCATATGTAATGAAATAGTGAATGGTTTTGAA
CTCTCATGGTTTACTTATGGATGTCAAAAATGCAAATCTGATGAGGTGTGCGACACGTCGTACGACTCTGACA
TCCCACCGGAGCTCAAGACTATCAAGTGCAGTAGGCATTCCTCAGGAGAACGCTGGTATGACCCGATAAAGAA
TAAGTTTTCAGGGAAACTTCTCATATATAACTCTAAATTTCATGTTCAGCTCCTTCTAAATGCACCCGTTTAT
TACGCAATACTACAAGCAGCTAAACTTGTAATTGGCATTCCTTGTTTAACTGCATTACTGATATACAAGTGGA
GGAGAAGACATTTGTCGATGTACGACGATATAGAAGACTTCCTGCGGACTAATAATAATCTCATGCCAGTGAG
ATACACTTACTCAGACATCAAGAAGATGACAAGGGATTTCAAGGAAAAATTGGGTCAAGGAGGCTATGGGACG
GTATACAAGGCAACCCTTCGTAGTGGTCGGCTTGTAGCCATTAAGATGTTGGGTAAGTCCCAAGCTAACGGGC
AAGACTTCATCAATGAAGTCGCGACCATTGGAAGGATTCACCATGTTAATGTTGTGCAACTAATTGGATTTTG
TGTTGAGGGATCCAAGCGTGCTCTTGTGTATGATTTCATGCCTAATGGGTCTCTTGACAAGTACATTTTTCTT
CCACAAGGAGTGAGCACCTTAAGTTTCAAGAAATTTTTTGAAATTGCGCTTGGAGTTGCTCGTGGTATCGACT
ATCTGCATCGAGGGTGTGATATGCAAATTTTGCACTTTGACATCAAGCCTCATAACATTCTTCTGGACGAGAA
TTTCATTCCCAAGGTTTCTGATTTTGGGTTAGCAAGGCTATACCCATTGGATAATAGCATCGTGTCTCTGACT
GCTGTAAGAGGCACGATAGGATACATAGCTCCTGAGCTATTCTATAAGAACATTGGAGGTGTTTCCTACAAGG
CTGATGTATATAGTTTTGGAATGCTATTGATGGAAATGGCTGGGAGAAGGAAGAACTTGAATGCAGGGATAGA
ATATTCAAGCCAATTTAGTGAAAATTACTTTCCTTCATGGCCCAGTGAACGCCCTTCAATGACAAAAGTAGTC
GAGATGCTTGAAGGAGAACTAGTGAGCCTCCAAATACCCCCAAAGCCTTTCTTATATCCACAACAAATGCCAG
TAGAGGATATTGGAGACACTTCTTCTACTACCGATGGATCGACAATGATGGAAGTGTCAACAGAGATCAACTG
A
163.26 amino acid sequence (SEQ ID NO: 4)
MSRRSLLFASYTILLLL?FTVISQSSDTGNPLDICTSSCGGIYISYPFRLKGDPQNCGNEKFELSCEANSTTA
VLYLESEEYYVKSIHYNNFTIRIVDSGVQKKRNYCSTGLHSMTTFDSSDPYSFSSLYTPYMEQVPIILVTCAT
PVNSTLFIETASCLNSTTIDSSKYNYFMVGSVTSFDLKSSCEVTQMLLTSPSINNSFLMSCESICNEIVNGFE
LSWFTYGCQKCKSDEVCDTSYDSDIPPELKTIKCSRHSSGERWYDPIKNKFSGKLLIYNSKFHVQLLLNAPVY
YAISQAAKLVIGIPCLTALLIYKWRRRHLSMYDDIEDFLRTNNNLMPVRYTYSDIKKMTRDFKEKSGQGGYGT
VYKATLRSGRLVAIKMLGKSQANGQDFINEVATIGRIHHVNVVQLIGFCVEGSKRALVYDEMPNGSLDKYIFL
PQGVSTLSFKKFFEIALGVARGIDYLHRGCDMQISHEDIKPHNILLDENFIPKVSDFGLARLYPLDNSIVSLT
AVRGTIGYIAPELFYKNIGGVSYKADVYSFGMLLMEMAGRRKNLNAGIEYSSQFSENYFPSWPSERPSMTKVV
EMLEGELVSLQIPPKPFLYPQQMPVEDIGDTSSTTDGSTMMEVSTEIN

1. A genetically modified Fragaria plant, wherein the genome of the Fragaria plant has been modified to express one or more AFR resistance genes at the FaRCa1 locus.

2. The genetically modified Fragaria plant of claim 1, wherein the Fragaria plant has been genetically modified to contain an insertion of:
(a) a heterologous 163.0 gene, coding sequence, or fragment thereof or a heterologous nucleic acid sequence encoding SEQ ID NO: 2; and/or
(b) a heterologous 163.26 gene, coding sequence, or fragment thereof or a heterologous nucleic acid sequence that encoding SEQ ID NO: 4.

3. The genetically modified Fragaria plant of claim 2, wherein the Fragaria plant has been genetically modified to contain an insertion of the SEQ ID NO: 1 and SEQ ID NO: 3, or the heterologous nucleic acid sequence encoding SEQ ID NO: 2 and the heterologous nucleic acid sequence encoding SEQ ID NO: 4.

4. The genetically modified Fragaria plant of any one of claims 1-3, wherein Fragaria plant has been genetically modified at the FaRCa1 locus.

5. The genetically modified Fragaria plant of any one of claims 2-4, wherein the genetically modified Fragaria plant has increased resistance to Anthracnose fruit rot (AFR) and/or anthracnose root necrosis (ARN) compared to a control Fragaria plant the doesn't express the heterologous 163.0 gene, coding sequence, or fragment thereof or the heterologous 163.26 gene, coding sequence, or fragment thereof.

6. A method of genetically modifying a Fragaria plant cell comprising: introducing one or more of a heterologous 163.0 gene, coding sequence, or fragment thereof, or a heterologous nucleic acid sequence encoding SEQ ID NO: 2, and/or a heterologous 163.26 gene, coding sequence, or fragment thereof, or a heterologous nucleic acid sequence encoding SEQ ID NO: 4 into the genome of the Fragaria plant cell through one or more of: unaided homologous recombination, recombinase-based insertion, and DNA repair-based insertion.

7. The method of claim 6, wherein the introducing the heterologous 163.0 gene, coding sequence, or fragment thereof, or the heterologous nucleic acid sequence encoding SEQ ID NO: 2, and/or the heterologous 163.26 gene, coding sequence, or fragment thereof, or the heterologous nucleic acid sequence encoding SEQ ID NO: 4 into the genome comprises a nuclease agent-mediated DNA repair-based insertion.

8. The method of claim 7, wherein the nuclease agent is a zinc finger nuclease (ZFN), a Transcription Activator-Like Effector Nuclease (TALEN), a meganuclease, or a Clustered Regularly Interspaced Short Palindromic Repeats (CRISPR)-associated (Cas) protein and a guide RNA (gRNA).

9. The method of claim 8, wherein the nuclease agent comprises the Cas protein and the gRNA.

10. The method of claim 9, wherein the Cas protein comprises a zCas9 nuclease, a Cas9 nuclease, type II Cas nuclease, an nCas9 nuclease, a type V Cas nuclease, a Cas12a nuclease, a Casl2b nuclease, a Casl2c nuclease, a CasY nuclease, a CasX nuclease, a Casl2i nuclease, or an engineered RNA-guided DNA endonuclease.

11. The method of claim 9, wherein the gRNA comprises a CRISPR RNA (crRNA) and a trans-activating CRISPR RNA (tracrRNA), as separate molecules or as a single chimeric guide RNA (sgRNA).

12. The method of any one of claims 6-11, wherein introducing the heterologous 163.0 gene, coding sequence, or fragment thereof, or the heterologous nucleic acid sequence encoding SEQ ID NO: 2, and/or the heterologous 163.26 gene, coding sequence, or fragment thereof, or the heterologous nucleic acid sequence encoding SEQ ID NO: 4 into a Fragaria plant cell comprises electroporation, microprojectile bombardment, biolistic transformation, microinjection, protoplast transformation, an Agrobacterium tumefaciens vector transformation or an Agrobacterium rhizogenes vector transformation.

13. The method of any one of claims 6-12, wherein the method further comprises generating one or more regenerants following introducing the heterologous 163.0 gene, coding sequence, or fragment thereof, or the heterologous nucleic acid sequence encoding SEQ ID NO: 2, and/or the heterologous 163.26 gene, coding sequence, or fragment thereof, or the heterologous nucleic acid sequence encoding SEQ ID NO: 4 into the Fragaria plant cell.

14. The method of claim 13, wherein the method further comprises genotyping the one or more regenerants for the presence of the heterologous 163.0 gene, coding sequence, or fragment thereof, or the heterologous nucleic acid sequence encoding SEQ ID NO: 2, and/or the heterologous 163.26 gene, coding sequence, or fragment thereof, or the heterologous nucleic acid sequence encoding SEQ ID NO: 4.

15. The method of claim 14, wherein the method further comprises selecting one or more To plants containing one or more of the heterologous 163.0 gene, coding sequence, or fragment thereof, or the heterologous nucleic acid sequence encoding SEQ ID NO: 2, and/or the heterologous 163.26 gene, coding sequence, or fragment thereof, or the heterologous nucleic acid sequence encoding SEQ ID NO: 4.

16. A method of genetically modifying a Fragaria plant at the FaRCa1 locus to produce a plant resistant to AFR and/or ARN, the method comprising: introducing a Cas protein or a nucleic acid encoding the Cas protein and a gRNA or a nucleic acid encoding the gRNA into a Fragaria plant cell, wherein the gRNA and Cas protein form a complex that targets the FaRCa1 locus to insert one or more of a heterologous 163.0 gene, coding sequence, or fragment thereof, or a heterologous nucleic acid sequence encoding SEQ ID NO: 2, and/or a heterologous 163.26 gene, coding sequence, or fragment thereof, or a heterologous nucleic acid sequence encoding SEQ ID NO: 4, and generating a regenerant plant from the Fragaria plant cell.

17. A method of genetically modifying a Fragaria plant, the method comprising:
introducing a CRISPR system into a Fragaria plant cell, wherein the CRISPR system comprises:
(a) an RNA-guided DNA endonuclease or a nucleic acid encoding the RNA-guided DNA endonuclease;
(b) a guide RNA or a nucleic acid encoding the guide RNA; and
(c) a DNA donor template containing a sequence encoding one or more of a heterologous 163.0 gene, coding sequence, or fragment thereof, or a heterologous nucleic acid sequence encoding SEQ ID NO: 2, and/or a heterologous 163.26 gene, coding sequence, or fragment thereof, or a heterologous nucleic acid sequence encoding SEQ ID NO: 4;
wherein the RNA-guided DNA endonuclease and the guide RNA form a complex that creates a single or double strand break in the genome of the Fragaria plant cell and the DNA donor template is used by the plant cell to repair the break and insert the heterologous 163.0 gene, coding sequence, or fragment thereof, or the heterologous nucleic acid sequence encoding SEQ ID NO: 2, and/or the heterologous 163.26 gene, coding sequence, or fragment thereof, or the heterologous nucleic acid sequence encoding SEQ ID NO: 4, into the genome at the site of the single or double strand break.

18. The method of claim 17, wherein the CRISPR system is selected from the group consisting of: a CRISPR class 1 system, a CRISPR class 2 system, a CRISPR/Cas system, a CRISPR/Cas9 system, a CRISPR/zCas9 system and a CRISPR/Cas3 system.

19. The method of claim 17 or 18, wherein the RNA-guided DNA endonuclease comprises a zCas9 protein.

20. The method of any one of claims 17-19, wherein the guide RNA comprises a CRISPR RNA (crRNA) and a trans-activating CRISPR RNA (tracrRNA) as separate molecules or as a single chimeric guide RNA (sgRNA).

21. The method of any one of claims 17-20, wherein introducing a CRISPR system into a Fragaria plant cell comprises electroporation, microprojectile bombardment, biolistic transformation, microinjection, protoplast transformation, an Agrobacterium tumefaciens vector transformation or an Agrobacterium rhizogenes vector transformation.

22. The method of any one of claims 17-21, wherein the DNA donor template comprises SEQ ID NO: 1 or a fragment thereof or a complement thereof, and/or SEQ ID NO: 3 or a fragment thereof or a complement thereof.

23. The method of any one of claim 17-22, wherein CRISPR system targets the FaRCa1 locus.

24. The method of any one of claim 17-23, wherein the genetically modified Fragaria plant cell overexpresses the heterologous 163.0 gene, coding sequence, or fragment thereof, or the heterologous nucleic acid sequence encoding SEQ ID NO: 2, and/or the heterologous 163.26 gene, coding sequence, or fragment thereof, or the heterologous nucleic acid sequence encoding SEQ ID NO: 4.

25. The method of any one of claims 17-24, wherein the method further comprises generating one or more regenerants following introducing the CRISPR system into a Fragaria plant cell.

26. The method of claim 25, wherein the method further comprises analyzing one or more regenerants for expression of the heterologous 163.0 gene, coding sequence, or fragment thereof, or the heterologous nucleic acid sequence encoding SEQ ID NO: 2, and/or the heterologous 163.26 gene, coding sequence, or fragment thereof, or the heterologous nucleic acid sequence encoding SEQ ID NO: 4.

27. The method of claim 25, wherein the method further comprises selecting one or more T₀ plants expressing the heterologous 163.0 gene, coding sequence, or fragment thereof, or the heterologous nucleic acid sequence encoding SEQ ID NO: 2, and/or the heterologous 163.26 gene, coding sequence, or fragment thereof, or the heterologous nucleic acid sequence encoding SEQ ID NO: 4.

28. The method of any one of claims 25-27, wherein the regenerant or the To plant has increased resistance to AFR and/or ARN.

29. A method of introgressing AFR and/or ARN resistance into a Fragaria plant, comprising:
(a) crossing at least one donor Fragaria plant known express SEQ ID NO: 2 and/or SEQ ID NO: 4 with at least one recipient Fragaria plant in order to form a segregating population; and
(b) screening the segregating population with one or more FaRCa1 markers to determine if one or more Fragaria plants from said segregating population contains the expressed SEQ ID NO: 2 and/or SEQ ID NO: 4, wherein the one or more FaRCa1 markers comprise one or more detectable genetic markers linked, closely linked, tightly linked, or extremely tightly linked to any of SEQ ID NO: 1 and/or SEQ ID NO: 3.

30. The method of claim 29, wherein the FaRCa1 marker comprises a PCR amplification product, a single nucleotide polymorphism (SNP), a restriction fragment length polymorphism (RFLP), an amplified fragment length polymorphism (AFLP), a simple sequence repeat (SSR), a simple sequence length polymorphism (SSLP), an insertion/deletion polymorphism (indel), a variable number tandem repeat (VNTRs), or a random amplified polymorphic DNA (RAPD).

31. The method of claim 30, wherein the PCR amplification product comprises all or a portion of SEQ ID NO: 1 or SEQ ID NO: 3.

32. The method of claim 29, wherein the FaRCa1 marker is in a continuous nucleic acid region comprising an interval between 16,272,461 and 16,273.939 bp on Chromosome 6-3 of the Fragaria×ananassa plant.

33. The method of claim 29, wherein screening the segregating population comprises amplifying a nucleotide sequence containing the FaRCa1 marker to produce an amplification product, wherein the amplification product is determinative for the presence and/or absence of the FaRCa1 marker.

34. The genetically modified Fragaria plant of any one of claims 1-5, of the method of any one of claims 6-33, wherein the Fragaria plant is an octoploid Fragaria plant.
